(19) United States
(12) Patent Application Publication
(10) Pub. No.: US 2008/0096211 A1
(43) Pub. Date: Apr. 24, 2008

(54) Corynebacterium glutamicum genes encoding proteins involved in genetic stability, gene expression, and protein secretion and folding

(75) Inventors: Markus Pompejus, Freinsheim (DE); Burkhard Kroger, Limburgerhof (DE); Hartwig Schroder, Nussloch (DE); Oskar Zelder, Speyer (DE); Gregor Haberhauer, Limburgerhof (DE)

Correspondence Address: LAHIVE & COCKFIELD, LLP, ONE POST OFFICE SQUARE, BOSTON, MA, 02109-2127, US

(73) Assignee: BASF Aktiengesellschaft, Ludwigshafen (DE)

(21) Appl. No.: 11/890,181

(22) Filed: Aug. 3, 2007

Related U.S. Application Data
(62) Division of application No. 11/041,504, filed on Jan. 21, 2005.
Application No. 11/041,504 is a continuation of application No. 09/602,839, filed on Jun. 23, 2000, now abandoned.
(60) Provisional application No. 60/143,752, filed on Jul. 14, 1999.
(60) Provisional application No. 60/151,671, filed on Aug. 31, 1999.
(60) Provisional application No. 60/141,031, filed on Jun. 25, 1999.

(30) Foreign Application Priority Data
Jul. 8, 1999 (DE) 19931412.8
Jul. 14, 1999 (DE) 19932928.1

(51) Int. Cl. C12P 21/04; C07K 14/00; C12N 1/20; C12N 15/00; C12N 15/11; C12P 13/04; C12P 13/06; C12P 13/08; C12P 7/64; G01N 33/53; C12Q 1/68; C12P 13/10; C12P 13/12; C12P 13/14; C12P 13/20; C12P 13/22; C12P 19/20; C12P 19/38

(52) U.S. Cl. 435/6; 435/106; 435/108; 435/109; 435/110; 435/113; 435/114; 435/115; 435/116; 435/134; 435/252.3; 435/252.32; 435/320.1; 435/69.1; 435/7.32; 435/87; 435/89; 530/350; 536/23.7

(57) ABSTRACT

Isolated nucleic acid molecules, designated SES nucleic acid molecules, which encode novel SES proteins from Corynebacterium glutamicum are described. The invention also provides antisense nucleic acid molecules, recombinant expression vectors containing SES nucleic acid molecules, and host cells into which the expression vectors have been introduced. The invention still further provides isolated SES proteins, mutated SES proteins, fusion proteins, antigenic peptides and methods for the improvement of production of a desired compound from C. glutamicum based on genetic engineering of SES genes in this organism.

### RELATED APPLICATIONS

This application is a divisional of U.S. application Ser. No. 11/041,504, filed Jan. 21, 2005, which is a continuation of U.S. application Ser. No. 09/602,839, filed Jun. 23, 2000, which, in turn, claims priority to prior filed U.S. Provisional Patent Application Ser. No. 60/141,031, filed Jun. 25, 1999, U.S. Provisional Patent Application Ser. No. 60/143,752, filed Jul. 14, 1999, and U.S. Provisional Patent Application Ser. No. 60/151,671, filed Aug. 31, 1999. This application also claims priority to prior filed German Patent Application No. 19931412.8, filed Jul. 8, 1999, and German Patent Application No. 19932928.1, filed Jul. 14, 1999. The entire contents of all of the aforementioned applications are expressly incorporated herein by this reference.

### INCORPORATION OF MATERIAL SUBMITTED ON COMPACT DISCS

This application incorporates herein by reference the material contained on the compact discs submitted herewith as part of this application. Specifically, the file “seqlist” (1.96 MB) contained on each of Copy 1 and Copy 2 of the Sequence Listing is hereby incorporated herein by reference. This file was created on Jul. 30, 2007. In addition, the files “Appendix A” (324 KB) and “Appendix B” (114 KB) contained on each of the compact discs entitled “Appendices Copy 1” and “Appendices Copy 2” are hereby incorporated herein by reference. Each of these files were created on Jul. 31, 2006.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, diols, carbohydrates, aromatic compounds, vitamins and cofactors, and enzymes. Their production is most conveniently performed through the large-scale culture of bacteria developed to produce and secrete large quantities of one or more desired molecules. One particularly useful organism for this purpose is Corynebacterium glutamicum, a gram positive, nonpathogenic bacterium. Through strain selection, a number of mutant strains have been developed which produce an array of desirable compounds. However, selection of strains improved for the production of a particular molecule is a time-consuming and difficult process.

### SUMMARY OF THE INVENTION

The invention provides novel bacterial nucleic acid molecules which have a variety of uses. These uses include the identification of microorganisms which can be used to produce fine chemicals, the modulation of fine chemical production in C. glutamicum or related bacteria, the typing or identification of C. glutamicum or related bacteria, as reference points for mapping the C. glutamicum genome, and as markers for transformation. These novel nucleic acid molecules encode proteins, referred to herein as stability, gene expression, or protein secretion/folding (SES) proteins.

C. glutamicum is a gram positive, aerobic bacterium which is commonly used in industry for the large-scale production of a variety of fine chemicals, and also for the degradation of hydrocarbons (such as in petroleum spills) and for the oxidation of terpenoids. The SES nucleic acid molecules of the invention, therefore, can be used to identify microorganisms which can be used to produce fine chemicals, e.g., by fermentation processes. Modulation of the expression of the SES nucleic acids of the invention, or modification of the sequence of the SES nucleic acid molecules of the invention, can be used to modulate the production of one or more fine chemicals from a microorganism (e.g., to improve the yield or production of one or more fine chemicals from a Corynebacterium or Brevibacterium species).

The SES nucleic acids of the invention may also be used to identify an organism as being Corynebacterium glutamicum or a close relative thereof, or to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is nonpathogenic, it is related to species pathogenic in humans, such as Corynebacterium diphtheriae (the causative agent of diphtheria); the detection of such organisms is of significant clinical relevance.

The SES nucleic acid molecules of the invention may also serve as reference points for mapping of the C. glutamicum genome, or of genomes of related organisms. Similarly, these molecules, or variants or portions thereof, may serve as markers for genetically engineered Corynebacterium or Brevibacterium species.

The SES proteins encoded by the novel nucleic acid molecules of the invention are capable of, for example, performing a function involved in the repair or recombination of DNA, transposition of genetic material, expression of genes (i.e., involved in transcription or translation), protein folding, or protein secretion in Corynebacterium glutamicum. Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

There are a number of mechanisms by which the alteration of an SES protein of the invention may directly affect the yield, production, and/or efficiency of production of a fine chemical from a C. glutamicum strain incorporating such an altered protein. For example, modulation of proteins involved directly in transcription or translation (e.g., polymerases or ribosomes) such that they are increased in number or in activity should increase global cellular transcription or translation (or rates of these processes). This increased cellular gene expression should include those proteins involved in fine chemical biosynthesis, so an increase in yield, production, or efficiency of production of one or more desired compounds may occur. Modifications to the transcriptional/translational protein machinery of C. glutamicum such that the regulation of these proteins is altered may also permit increased expression of genes involved in the production of fine chemicals. Modulation of the activity or number of proteins involved in polypeptide folding may permit an increase in the overall production of correctly folded molecules in the cell, thereby increasing the possibility that desired proteins (e.g., fine chemical biosynthetic proteins) are able to function properly. Further, by mutating proteins involved in secretion from C. glutamicum such that they are increased in number or activity, it may be possible to increase the secretion of a fine chemical (e.g., an enzyme) from cells in fermentor culture, where it may be readily recovered.

Genetic modification of the SES molecules of the invention may also result in indirect modulation of production of one or more fine chemicals. For example, by increasing the number or activity of a DNA repair or recombination protein of the invention, one may increase the ability of the cell to detect and repair DNA damage. This should effectively increase the ability of the cell to maintain a mutated gene within its genome, thereby increasing the likelihood that a transgene engineered into C. glutamicum (e.g., encoding a protein which will increase biosynthesis of a fine chemical) will not be lost during culture of the microorganism. Conversely, by decreasing the number or activity of one or more DNA repair or recombination proteins, it may be possible to increase the genetic instability of the organism. Such manipulations should improve the ability of the organism to be modified by mutagenesis without the introduced mutation being corrected. The same holds true for proteins involved in transposition or rearrangement of genetic elements in C. glutamicum (e.g., transposons). By mutagenizing these proteins such that they are either increased or decreased in number or activity, it is possible to simultaneously increase or decrease the genetic stability of the microorganism. This has a profound impact on the ability of any other mutation to be introduced into C. glutamicum, and on the ability of introduced mutations to be retained. Transposons also offer a convenient mechanism by which mutagenesis of C. glutamicum may be performed; duplication of desired genes (e.g., fine chemical biosynthetic genes) is readily accomplished by transposon mutagenesis, as is disruption of undesired genes (e.g., genes encoding proteins involved in degradation of desired fine chemicals).

By modulating one or more proteins (e.g. sigma factors) involved in the regulation of transcription or translation in response to particular environmental conditions, it may be possible to prevent the cell from slowing or stopping protein synthesis under unfavorable environmental conditions, such as those found in large-scale fermentor culture. This should lead to increased gene expression, which in turn may permit increased biosynthesis of desired fine chemicals under such conditions. Mutagenesis of proteins involved in protein secretion systems may result in modulated secretion rates. Many such secreted proteins have functions critical for cell viability (e.g., cell surface proteases or receptors). An alteration of a secretory pathway such that these proteins are more readily transported to their extracellular location may improve the overall viability of the cell, and thus result in greater numbers of C. glutamicum cells capable of producing fine chemicals during large-scale culture. Further, the secretion apparatus (e.g., the sec system) is also known to be involved in the insertion of integral membrane proteins (e.g., pores, channels, or transporters) into the membrane. Thus, the modulation of activity of proteins involved in protein secretion from C. glutamicum may affect the ability of the cell to excrete waste products or to import necessary metabolites. If the activity of these secretory proteins is increased, then the ability of the cell to produce fine chemicals may be similarly increased. If the activity of these secretory proteins is decreased, then there may be insufficient nutrients available to support overproduction of desired compounds, or waste products may interfere with such biosynthesis.

The invention provides novel nucleic acid molecules which encode proteins, referred to herein as SES proteins, which are capable of, for example, participating in the repair or recombination of DNA, transposition of genetic material, expression of genes (i.e., the processes of transcription or translation), protein folding, or protein secretion in Corynebacterium glutamicum. Nucleic acid molecules encoding an SES protein are referred to herein as SES nucleic acid molecules. In a preferred embodiment, an SES protein participates in improving or decreasing genetic stability in C. glutamicum, in the expression of genes (i.e., in transcription or translation) or protein folding in this organism, or in protein secretion from C. glutamicum. Examples of such proteins include those encoded by the genes set forth in Table 1.

Accordingly, one aspect of the invention pertains to isolated nucleic acid molecules (e.g., cDNAs, DNAs, or RNAs) comprising a nucleotide sequence encoding an SES protein or biologically active portions thereof, as well as nucleic acid fragments suitable as primers or hybridization probes for the detection or amplification of SES-encoding nucleic acid (e.g., DNA or mRNA). In particularly preferred embodiments, the isolated nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A, or a portion thereof. In other preferred embodiments, the isolated nucleic acid molecule encodes one of the amino acid sequences set forth in Appendix B. The preferred SES proteins of the present invention also preferably possess at least one of the SES activities described herein.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an SES activity. Preferably, the protein or portion thereof encoded by the nucleic acid molecule maintains the ability to participate in the repair or recombination of DNA, in the transposition of genetic material, in gene expression (i.e., the processes of transcription or translation), in protein folding, or in protein secretion in Corynebacterium glutamicum. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length C. glutamicum protein which is substantially homologous to an entire amino acid sequence of Appendix B (encoded by an open reading frame shown in Appendix A).

In another preferred embodiment, the isolated nucleic acid molecule is derived from C. glutamicum and encodes a protein (e.g., an SES fusion protein) which includes a biologically active domain which is at least about 50% or more homologous to one of the amino acid sequences of Appendix B and is able to participate in the repair or recombination of DNA, in the transposition of genetic material, in gene expression (i.e., the processes of transcription or translation), in protein folding, or in protein secretion in Corynebacterium glutamicum, or has one or more of the activities set forth in Table 1, and which also includes heterologous nucleic acid sequences encoding a heterologous polypeptide or regulatory regions.

In another embodiment, the isolated nucleic acid molecule is at least 15 nucleotides in length and hybridizes under stringent conditions to a nucleic acid molecule comprising a nucleotide sequence of Appendix A. Preferably, the isolated nucleic acid molecule corresponds to a naturally-occurring nucleic acid molecule. More preferably, the isolated nucleic acid encodes a naturally-occurring C. glutamicum SES protein, or a biologically active portion thereof.

Another aspect of the invention pertains to vectors, e.g., recombinant expression vectors, containing the nucleic acid molecules of the invention, and host cells into which such vectors have been introduced. In one embodiment, such a host cell is used to produce an SES protein by culturing the host cell in a suitable medium. The SES protein can be then isolated from the medium or the host cell.

Yet another aspect of the invention pertains to a genetically altered microorganism in which an SES gene has been introduced or altered. In one embodiment, the genome of the microorganism has been altered by introduction of a nucleic acid molecule of the invention encoding wild-type or mutated SES sequence as a transgene. In another embodiment, an endogenous SES gene within the genome of the microorganism has been altered, e.g., functionally disrupted, by homologous recombination with an altered SES gene. In another embodiment, an endogenous or introduced SES gene in a microorganism has been altered by one or more point mutations, deletions, or inversions, but still encodes a functional SES protein. In still another embodiment, one or more of the regulatory regions (e.g., a promoter, repressor, or inducer) of an SES gene in a microorganism has been altered (e.g., by deletion, truncation, inversion, or point mutation) such that the expression of the SES gene is modulated. In a preferred embodiment, the microorganism belongs to the genus Corynebacterium or Brevibacterium, with Corynebacterium glutamicum being particularly preferred. In a preferred embodiment, the microorganism is also utilized for the production of a desired compound, such as an amino acid, with lysine being particularly preferred.

In another aspect, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject.

Still another aspect of the invention pertains to an isolated SES protein or a portion, e.g., a biologically active portion, thereof. In a preferred embodiment, the isolated SES protein or portion thereof can participate in the repair or recombination of DNA, in the transposition of genetic material, in gene expression (i.e., the processes of transcription or translation), in protein folding, or in protein secretion in Corynebacterium glutamicum. In another preferred embodiment, the isolated SES protein or portion thereof is sufficiently homologous to an amino acid sequence of Appendix B such that the protein or portion thereof maintains the ability to participate in the repair or recombination of DNA, in the transposition of genetic material, in gene expression (i.e., the processes of transcription or translation), in protein folding, or in protein secretion in Corynebacterium glutamicum.

The invention also provides an isolated preparation of an SES protein. In preferred embodiments, the SES protein comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated SES protein comprises an amino acid sequence which is at least about 50% or more homologous to one of the amino acid sequences of Appendix B and is able to participate in the repair or recombination of DNA, in the transposition of genetic material, in gene expression (i.e., the processes of transcription or translation), in protein folding, or in protein secretion in Corynebacterium glutamicum, or has one or more of the activities set forth in Table 1.

Alternatively, the isolated SES protein can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of SES proteins also have one or more of the SES bioactivities described herein.

The SES polypeptide, or a biologically active portion thereof, can be operatively linked to a non-SES polypeptide to form a fusion protein. In preferred embodiments, this fusion protein has an activity which differs from that of the SES protein alone. In other preferred embodiments, this fusion protein participates in the repair or recombination of DNA, in the transposition of genetic material, in gene expression (i.e., the processes of transcription or translation), in protein folding, or in protein secretion in Corynebacterium glutamicum. In particularly preferred embodiments, integration of this fusion protein into a host cell modulates production of a desired compound from the cell.

In another aspect, the invention provides methods for screening molecules which modulate the activity of an SES protein, either by interacting with the protein itself or a substrate or binding partner of the SES protein, or by modulating the transcription or translation of an SES nucleic acid molecule of the invention.

Another aspect of the invention pertains to a method for producing a fine chemical. This method involves the culturing of a cell containing a vector directing the expression of an SES nucleic acid molecule of the invention, such that a fine chemical is produced. In a preferred embodiment, this method further includes the step of obtaining a cell containing such a vector, in which a cell is transfected with a vector directing the expression of an SES nucleic acid. In another preferred embodiment, this method further includes the step of recovering the fine chemical from the culture. In a particularly preferred embodiment, the cell is from the genus Corynebacterium or Brevibacterium, or is selected from those strains set forth in Table 3.

Another aspect of the invention pertains to methods for modulating production of a molecule from a microorganism. Such methods include contacting the cell with an agent which modulates SES protein activity or SES nucleic acid expression such that a cell associated activity is altered relative to this same activity in the absence of the agent. In a preferred embodiment, the cell is modulated for one or more C. glutamicum processes involved in genetic stability, gene expression, protein folding, or protein secretion such that the yield, production, or efficiency of production of a desired fine chemical by this microorganism is improved. The agent which modulates SES protein activity can be an agent which stimulates SES protein activity or SES nucleic acid expression. Examples of agents which stimulate SES protein activity or SES nucleic acid expression include small molecules, active SES proteins, and nucleic acids encoding SES proteins that have been introduced into the cell. Examples of agents which inhibit SES activity or expression include small molecules and antisense SES nucleic acid molecules.

Another aspect of the invention pertains to methods for modulating yields of a desired compound from a cell, involving the introduction of a wild-type or mutant SES gene into a cell, either maintained on a separate plasmid or integrated into the genome of the host cell. If integrated into the genome, such integration can be random, or it can take place by homologous recombination such that the native gene is replaced by the introduced copy, causing the production of the desired compound from the cell to be modulated. In a preferred embodiment, said yields are increased. In another preferred embodiment, said chemical is a fine chemical. In a particularly preferred embodiment, said fine chemical is an amino acid. In especially preferred embodiments, said amino acid is L-lysine.

### DETAILED DESCRIPTION OF THE INVENTION

The present invention provides SES nucleic acid and protein molecules which are involved in the repair or recombination of DNA, in the transposition of genetic material, in gene expression (i.e., the processes of transcription or translation), in protein folding, or in protein secretion in Corynebacterium glutamicum. The molecules of the invention may be utilized in the modulation of production of fine chemicals from microorganisms, such as C. glutamicum, either directly (e.g., where overexpression or optimization of activity of a protein involved in secretion of a fine chemical (e.g., an enzyme) has a direct impact on the yield, production, and/or efficiency of production of a fine chemical from the modified C. glutamicum), or an indirect impact which nonetheless results in an increase of yield, production, and/or efficiency of production of the desired compound (e.g., where modulation of the activity or number of copies of a C. glutamicum DNA repair protein results in alterations in the ability of the microorganism to maintain the introduced mutation, which in turn may impact the production of one or more fine chemicals from such a strain). Aspects of the invention are further explicated below.

I. Fine Chemicals

The term ‘fine chemical’ is art-recognized and includes molecules produced by an organism which have applications in various industries, such as, but not limited to, the pharmaceutical, agriculture, and cosmetics industries. Such compounds include organic acids, such as tartaric acid, itaconic acid, and diaminopimelic acid, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and unsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, “Vitamins”, p. 443-613 (1996) VCH: Weinheim and references therein; and Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press, (1995)), enzymes, polyketides (Cane et al. (1998) Science 282: 63-68), and all other chemicals described in Gutcho (1983) Chemicals by Fermentation, Noyes Data Corporation, ISBN: 0818805086 and references therein. The metabolism and uses of certain of these fine chemicals are further explicated below.

A. Amino Acid Metabolism and Uses

Amino acids comprise the basic structural units of all proteins, and as such are essential for normal cellular functioning in all organisms. The term “amino acid” is art-recognized. The proteinogenic amino acids, of which there are 20 species, serve as structural units for proteins, in which they are linked by peptide bonds, while the nonproteinogenic amino acids (hundreds of which are known) are not normally found in proteins (see Ulmann's Encyclopedia of Industrial Chemistry, vol. A2, p. 57-97 VCH: Weinheim (1985)). Amino acids may be in the D- or L-optical configuration, though L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 3^{rd} edition, pages 578-590 (1988)). The ‘essential’ amino acids (histidine, isoleucine, leucine, lysine, methionine, phenylalanine, threonine, tryptophan, and valine), so named because they are generally a nutritional requirement due to the complexity of their biosyntheses, are readily converted by simple biosynthetic pathways to the remaining 11 ‘nonessential’ amino acids (alanine, arginine, asparagine, aspartate, cysteine, glutamate, glutamine, glycine, proline, serine, and tyrosine). Higher animals do retain the ability to synthesize some of these amino acids, but the essential amino acids must be supplied from the diet in order for normal protein synthesis to occur.

Aside from their function in protein biosynthesis, these amino acids are interesting chemicals in their own right, and many have been found to have various applications in the food, feed, chemical, cosmetics, agriculture, and pharmaceutical industries. Lysine is an important amino acid in the nutrition not only of humans, but also of monogastric animals such as poultry and swine. Glutamate is most commonly used as a flavor additive (mono-sodium glutamate, MSG) and is widely used throughout the food industry, as are aspartate, phenylalanine, glycine, and cysteine. Glycine, L-methionine and tryptophan are all utilized in the pharmaceutical industry. Glutamine, valine, leucine, isoleucine, histidine, arginine, proline, serine and alanine are of use in both the pharmaceutical and cosmetics industries. Threonine, tryptophan, and D/L-methionine are common feed additives. (Leuchtenberger, W. (1996) Amino aids—technical production and use, p. 466-502 in Rehm et al. (eds.) Biotechnology vol. 6, chapter 14a, VCH: Weinheim). Additionally, these amino acids have been found to be useful as precursors for the synthesis of synthetic amino acids and proteins, such as N-acetylcysteine, S-carboxymethyl-L-cysteine, (S)-5-hydroxytryptophan, and others described in Ulmann's Encyclopedia of Industrial Chemistry, vol. A2, p. 57-97, VCH: Weinheim, 1985.

The biosynthesis of these natural amino acids in organisms capable of producing them, such as bacteria, has been well characterized (for review of bacterial amino acid biosynthesis and regulation thereof, see Umbarger, H. E. (1978) Ann. Rev. Biochem. 47: 533-606). Glutamate is synthesized by the reductive amination of α-ketoglutarate, an intermediate in the citric acid cycle. Glutamine, proline, and arginine are each subsequently produced from glutamate. The biosynthesis of serine is a three-step process beginning with 3-phosphoglycerate (an intermediate in glycolysis), and resulting in this amino acid after oxidation, transamidation, and hydrolysis steps. Both cysteine and glycine are produced from serine; the former by the condensation of homocysteine with serine, and the latter by the transferal of the side-chain β-carbon atom to tetrahydrofolate, in a reaction catalyzed by serine transhydroxymethylase. Phenylalanine, and tyrosine are synthesized from the glycolytic and pentose phosphate pathway precursors erythrose 4-phosphate and phosphoenolpyruvate in a 9-step biosynthetic pathway that differ only at the final two steps after synthesis of prephenate. Tryptophan is also produced from these two initial molecules, but its synthesis is an 11-step pathway. Tyrosine may also be synthesized from phenylalanine, in a reaction catalyzed by phenylalanine hydroxylase. Alanine, valine, and leucine are all biosynthetic products of pyruvate, the final product of glycolysis. Aspartate is formed from oxaloacetate, an intermediate of the citric acid cycle. Asparagine, methionine, threonine, and lysine are each produced by the conversion of aspartate. Isoleucine is formed from threonine. A complex 9-step pathway results in the production of histidine from 5-phosphoribosyl-1-pyrophosphate, an activated sugar.

Amino acids in excess of the protein synthesis needs of the cell cannot be stored, and are instead degraded to provide intermediates for the major metabolic pathways of the cell (for review see Stryer, L. Biochemistry 3^{rd} ed. Ch. 21 “Amino Acid Degradation and the Urea Cycle” p. 495-516 (1988)). Although the cell is able to convert unwanted amino acids into useful metabolic intermediates, amino acid production is costly in terms of energy, precursor molecules, and the enzymes necessary to synthesize them. Thus it is not surprising that amino acid biosynthesis is regulated by feedback inhibition, in which the presence of a particular amino acid serves to slow or entirely stop its own production (for overview of feedback mechanisms in amino acid biosynthetic pathways, see Stryer, L. Biochemistry, 3^{rd} ed. Ch. 24: “Biosynthesis of Amino Acids and Heme” p. 575-600 (1988)). Thus, the output of any particular amino acid is limited by the amount of that amino acid present in the cell.

B. Vitamin, Cofactor, and Nutraceutical Metabolism and Uses

Vitamins, cofactors, and nutraceuticals comprise another group of molecules which the higher animals have lost the ability to synthesize and so must ingest, although they are readily synthesized by other organisms such as bacteria. These molecules are either bioactive substances themselves, or are precursors of biologically active substances which may serve as electron carriers or intermediates in a variety of metabolic pathways. Aside from their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria, has been largely characterized (Ullman's Encyclopedia of Industrial Chemistry, “Vitamins” vol. A27, p. 443-613, VCH: Weinheim, 1996; Michal, G. (1999) Biochemical Pathways An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press: Champaign, Ill. X, 374 S).

Thiamin (vitamin B₁) is produced by the chemical coupling of pyrimidine and thiazole moieties. Riboflavin (vitamin B₂) is synthesized from guanosine-5′-triphosphate (GTP) and ribose-5′-phosphate. Riboflavin, in turn, is utilized for the synthesis of flavin mononucleotide (FMN) and flavin adenine dinucleotide (FAD). The family of compounds collectively termed ‘vitamin B₆’ (e.g., pyridoxine, pyridoxamine, pyridoxa-5′-phosphate, and the commercially used pyridoxin hydrochloride) are all derivatives of the common structural unit, 5-hydroxy-6-methylpyridine. Pantothenate (pantothenic acid, (R)-(+)-N-(2,4-dihydroxy-3,3-dimethyl-1-oxobutyl)-β-alanine) can be produced either by chemical synthesis or by fermentation. The final steps in pantothenate biosynthesis consist of the ATP-driven condensation of β-alanine and pantoic acid. The enzymes responsible for the biosynthesis steps for the conversion to pantoic acid, to β-alanine and for the condensation to panthotenic acid are known. The metabolically active form of pantothenate is Coenzyme A, for which the biosynthesis proceeds in 5 enzymatic steps. Pantothenate, pyridoxal-5′-phosphate, cysteine and ATP are the precursors of Coenzyme A. These enzymes not only catalyze the formation of panthothante, but also the production of (R)-pantoic acid, (R)-pantolacton, (R)-panthenol (provitamin B₅), pantetheine (and its derivatives) and coenzyme A.

Biotin biosynthesis from the precursor molecule pimeloyl-CoA in microorganisms has been studied in detail and several of the genes involved have been identified. Many of the corresponding proteins have been found to also be involved in Fe-cluster synthesis and are members of the nifS class of proteins. Lipoic acid is derived from octanoic acid, and serves as a coenzyme in energy metabolism, where it becomes part of the pyruvate dehydrogenase complex and the α-ketoglutarate dehydrogenase complex. The folates are a group of substances which are au derivatives of folic acid, which is turn is derived from L-glutamic acid, p-amino-benzoic acid and 6-methylpterin. The biosynthesis of folic acid and its derivatives, starting from the metabolism intermediates guanosine-5′-triphosphate (GTP), L-glutamic acid and p-amino-benzoic acid has been studied in detail in certain microorganisms.

Corrinoids (such as the cobalamines and particularly vitamin B₁₂) and porphyrines belong to a group of chemicals characterized by a tetrapyrole ring system. The biosynthesis of vitamin B₁₂ is sufficiently complex that it has not yet been completely characterized, but many of the enzymes and substrates involved are now known. Nicotinic acid (nicotinate), and nicotinamide are pyridine derivatives which are also termed ‘niacin’. Niacin is the precursor of the important coenzymes NAD (nicotinamide adenine dinucleotide) and NADP (nicotinamide adenine dinucleotide phosphate) and their reduced forms.

The large-scale production of these compounds has largely relied on cell-free chemical syntheses, though some of these chemicals have also been produced by large-scale culture of microorganisms, such as riboflavin, Vitamin B₆, pantothenate, and biotin. Only Vitamin B₁₂ is produced solely by fermentation, due to the complexity of its synthesis. In vitro methodologies require significant inputs of materials and time, often at great cost.

C. Purine, Pyrimidine, Nucleoside and Nucleotide Metabolism and Uses

Purine and pyrimidine metabolism genes and their corresponding proteins are important targets for the therapy of tumor diseases and viral infections. The language “purine” or “pyrimidine” includes the nitrogenous bases which are constituents of nucleic acids, co-enzymes, and nucleotides. The term “nucleotide” includes the basic structural units of nucleic acid molecules, which are comprised of a nitrogenous base, a pentose sugar (in the case of RNA, the sugar is ribose; in the case of DNA, the sugar is D-deoxyribose), and phosphoric acid. The language “nucleoside” includes molecules which serve as precursors to nucleotides, but which are lacking the phosphoric acid moiety that nucleotides possess. By inhibiting the biosynthesis of these molecules, or their mobilization to form nucleic acid molecules, it is possible to inhibit RNA and DNA synthesis; by inhibiting this activity in a fashion targeted to cancerous cells, the ability of tumor cells to divide and replicate may be inhibited. Additionally, there are nucleotides which do not form nucleic acid molecules, but rather serve as energy stores (i.e., AMP) or as coenzymes (i.e., FAD and NAD).

Several publications have described the use of these chemicals for these medical indications, by influencing purine and/or pyrimidine metabolism (e.g. Christopherson, R. I. and Lyons, S. D. (1990) “Potent inhibitors of de novo pyrimidine and purine biosynthesis as chemotherapeutic agents.” Med. Res. Reviews 10: 505-548). Studies of enzymes involved in purine and pyrimidine metabolism have been focused on the development of new drugs which can be used, for example, as immunosuppressants or anti-proliferants (Smith, J. L., (1995) “Enzymes in nucleotide synthesis.” Curr. Opin. Struct. Biol. 5: 752-757; (1995) Biochem Soc. Transact. 23: 877-902). However, purine and pyrimidine bases, nucleosides and nucleotides have other utilities: as intermediates in the biosynthesis of several fine chemicals (e.g., thiamine, S-adenosyl-methionine, folates, or riboflavin), as energy carriers for the cell (e.g., ATP or GTP), and for chemicals themselves, commonly used as flavor enhancers (e.g., IMP or GMP) or for several medicinal applications (see, for example, Kuninaka, A. (1996) Nucleotides and Related Compounds in Biotechnology vol. 6, Rehm et al., eds. VCH: Weinheim, p. 561-612). Also, enzymes involved in purine, pyrimidine, nucleoside, or nucleotide metabolism are increasingly serving as targets against which chemicals for crop protection, including fungicides, herbicides and insecticides, are developed.

The metabolism of these compounds in bacteria has been characterized (for reviews see, for example, Zalkin, H. and Dixon, J. E. (1992) “de novo purine nucleotide biosynthesis”, in: Progress in Nucleic Acid Research and Molecular Biology, vol. 42, Academic Press:, p. 259-287; and Michal, G. (1999) “Nucleotides and Nucleosides”, Chapter 8 in: Biochemical Pathways: An Atlas of Biochemistry and Molecular Biology, Wiley: New York). Purine metabolism has been the subject of intensive research, and is essential to the normal functioning of the cell. Impaired purine metabolism in higher animals can cause severe disease, such as gout. Purine nucleotides are synthesized from ribose-5-phosphate, in a series of steps through the intermediate compound inosine-5′-phosphate (IMP), resulting in the production of guanosine-5′-monophosphate (GMP) or adenosine-5′-monophosphate (AMP), from which the triphosphate forms utilized as nucleotides are readily formed. These compounds are also utilized as energy stores, so their degradation provides energy for many different biochemical processes in the cell. Pyrimidine biosynthesis proceeds by the formation of uridine-5′-monophosphate (UMP) from ribose-5-phosphate. UMP, in turn, is converted to cytidine-5′-triphosphate (CTP). The deoxy-forms of all of these nucleotides are produced in a one step reduction reaction from the diphosphate ribose form of the nucleotide to the diphosphate deoxyribose form of the nucleotide. Upon phosphorylation, these molecules are able to participate in DNA synthesis.

D. Trehalose Metabolism and Uses

Trehalose consists of two glucose molecules, bound in α, α-1,1 linkage. It is commonly used in the food industry as a sweetener, an additive for dried or frozen foods, and in beverages. However, it also has applications in the pharmaceutical, cosmetics and biotechnology industries (see, for example, Nishimoto et al., (1998) U.S. Pat. No. 5,759,610; Singer, M. A. and Lindquist, S. (1998) Trends Biotech. 16: 460-467; Paiva, C. L. A. and Panek, A. D. (1996) Biotech. Ann. Rev. 2: 293-314; and Shiosaka, M. (1997) J. Japan 172: 97-102). Trehalose is produced by enzymes from many microorganisms and is naturally released into the surrounding medium, from which it can be collected using methods known in the art.

II. Genetic Stability: Protein Synthesis and Protein Secretion in C. glutamicum

The production of a desired compound from a cell such as C. glutamicum is the culmination of a large number of separate yet interrelated processes, each of which is critical to the overall production and release of the compound from the cell. In engineering a cell to overproduce one or more fine chemicals, consideration must be given to each of these processes to ensure that the biochemical machinery of the cell will be compatible with such genetic manipulation. Cellular mechanisms of particular importance include the stability of the altered gene(s) upon introduction into the cell, the ability of the mutated gene to be properly transcribed and translated (including issues of codon usage), and the ability of the mutant protein product to be appropriately folded and/or secreted.

A. Bacterial Repair and Recombination Systems

Cells are constantly exposed to nucleic acid-damaging agents, such as UV irradiation, oxygen radicals, and alkylation. Further, even the action of DNA polymerases is not error-free. Cells must maintain a balance between genetic stability (which ensures that genes necessary for vital cellular functions are not damaged during normal growth and metabolism) and genetic variability (which permits cells to adapt to a changing environment). Therefore, there exist separate, but interrelated pathways of DNA repair and DNA recombination in most cells. The former serves to stringently correct errors in DNA molecules by either directly reversing the damage or excising the damaged region and replacing it with the correct sequence. The latter recombination system also repairs nucleic acid molecules, but only those lesions that result in damage to both strands of DNA such that neither strand is able to serve as a template to correct the other. Recombination repair and the SOS response may readily lead to inversions, deletions, or other genetic rearrangements within or around the region of the damage, which in turn promotes a certain degree of genomic instability which may contribute to the ability of the cell to adapt to changing environments or stresses.

High-fidelity repair mechanisms include direct reversal of DNA damage and excision of damage and resynthesis using the information encoded on the opposite DNA strand. Direct reversal of damage requires an enzyme having an activity opposite of that which originally damaged the DNA. For example, inappropriate methylation of DNA may be corrected by the action of DNA repair methyltransferases, and nucleotide dimers created by UV irradiation may be fixed by the activity of deoxyribodipyrimidine photo-lyase, which, in the presence of light, cleaves the dimer back to its constituent nucleotides (see Michal, G. (1999) Biochemical Pathways: An Atlas of Biochemistry and Molecular Biology, Wiley: New York, and references therein).

Precise repair of more extensive damage requires specialized repair mechanisms. These include the mismatch repair and excision repair systems. Damage to a single base may be corrected by a series of cleavage reactions, where first the sugar-base bond is cut, followed by cleavage of the DNA backbone at the site of damage and removal of the damaged base itself Finally, DNA polymerase and DNA ligase act to fill in and seal the gap using the second DNA strand as a template. More significant DNA damage which results in altered conformation of the double helix is corrected by the ABC system, in which helicase II, DNA polymerase I, UvrA, UvrB, and UvrC proteins combine to nick the double helix at the site of damage, to unwind the damaged region in an ATP-dependent fashion, to excise the damaged region, and to fill in the missing region using the other strand as a template. Lastly, DNA ligase seals the nick. Specific repair systems also exist for G-T mismatches (involving the Vsr protein) and for small deletion/insertion errors resulting in mispairing of the two strands (involving the methylation-directed pathway).

There also exist low-fidelity repair systems which are generally used to correct very extensive DNA damage in bacteria. Double-strand repair and recombination occurs in the presence of a lesion which affects both strands of DNA. In this situation, it is impossible to repair the damage utilizing the other strand as the template. Thus, this repair system involves a double-crossover event between the area of the lesion and another copy of the region on a homologous DNA molecule. This is possible because bacteria divide so rapidly that a second copy of genomic DNA is usually available before actual cell division occurs. This crossover event may readily lead to inversions, duplications, deletions, insertions and other genetic rearrangements, and thus increases the overall genetic instability of the organism.

The SOS response is activated when sufficient damage is present in the DNA that DNA polymerase III stalls and cannot continue replication. Under these circumstances, single-stranded DNA is present. The RecA protein is activated by binding to single-stranded DNA, and this activated form results in the activation of the LexA repressor, thereby lifting the transcriptional block of more than 20 genes, including UvrA, UvrB, UvrC, helicase II, DNA pol III, UmuC, and UmuD. The combined activities of these enzymes results in sufficient filling of the gap region that DNA pol III is able to resume replication. However, these gaps have been filled in with bases which should not be present; thus, this type of repair results in error-prone repair, contributing to overall genetic instability in the cell.

B. Transposons

The aforementioned systems, whether high or low fidelity, exist to repair DNA damage. In certain circumstances, this repair may accidentally incorporate additional genetic rearrangements. Many bacterial cells also have mechanisms specifically designed to cause such genetic rearrangements. Particularly well-known examples of such mechanisms are the transposons.

Transposons are genetic elements which are able to move from one site to another either within a chromosome or between a piece of extrachromosomal DNA (e.g., a plasmid) and a chromosome. Transposition may occur in multiple ways; for example, the transposable element may be cut out from the donor site and integrated into the target site (nonreplicative transposition), or the transposable element may alternately be duplicated from the donor site to the target site, yielding two copies of the element (replicative transposition). There is generally no sequence relationship between the donor and target sites.

There are a variety of results possible from such a transposition event. The integration of a transposable element into a gene disrupts the gene, usually abrogating its function entirely. An integration event that occurs in the DNA surrounding a gene may not perturb the coding sequence itself, but can have a profound effect on the regulation of the gene and thus, on its expression. Recombination events between two copies of a transposable element found in different portions of the genome may result in deletions, duplications, inversions, transpositions, or amplifications of segments of the genome. It is also possible for different replicons to fuse.

The simplest transposon-like genetic elements are termed insertion (IS) elements. IS elements contain a nucleotide region of varying length (though usually less than 1500 bases) lacking any coding regions, surrounded by inverted repeats at either end. Thus, since the IS element does not encode any proteins whose activity may be detected, the presence of an IS element is generally only observed due to a loss of function of one or more genes in which the IS element is inserted.

Transposons are mobile genetic elements which, unlike IS elements, contain nucleic acid sequences bounded by repeats which may encode one or more proteins. It is not unusual for these repeat regions to consist of IS elements. The proteins encoded by the transposon are typically transposases (proteins which catalyze the movement of the transposon from one site to another) and antibiotic resistance genes. The mechanisms and regulation of transposable elements are well known in the art and are have been described at least in, for example, Lengeler et al. (1999) Biology of Prokaryotes, Thieme Verlag: Stuttgart, p. 375-361; Neidhardt et al. (996) Escherichia coli and Salmonella, ASM Press: Washington, D.C.; Sonenshein, A. L. et al., eds. (1993), Bacillus subtilis, ASM Press: Washington, D.C.; Voet, D. and Voet, J. G. (1992) Biochemie, VCH: Weinheim, p. 985-990; Brock, T. D., and Madigan, M. T. (1991) Biology of Microorganisms, 6^{th} ed., Prentice Hall: New York, p. 267-269; and Kleckner, N. (1990) “Regulation of transposition in bacteria”, Annu. Rev. Biochem. 61: 297-327.

C. Transcription

Gene expression in bacteria is regulated mainly at the level of transcription. The transcriptional apparatus consists of a number of proteins that can be divided into two groups: RNA polymerase (the processive DNA-transcribing enzyme) and sigma factors (which regulate gene transcription by directing RNA polymerase to specific promoter-DNA sequences which these factors recognize). The combination of RNA polymerase and sigma factors creates the RNA polymerase holoenzyme, an activated complex. Gram positive bacteria such as Corynebacteria contain only one type of RNA-polymerase, but a variety of different sigma factors specific for different promoters, growth phases, environmental conditions, substrates, oxygen levels, transport processes, and the like, which permits adaptability of the organism to different environmental and metabolic conditions.

Promoters are specific DNA sequences that serve as docking sites for the RNA polymerase holoenzyme. Many promoter elements possess conserved sequence elements that may be recognized through homology searches; alternately, promoter regions for a particular gene may be identified using standard techniques such as primer extension. Many promoter regions from gram-positive bacteria are known (see, e.g., Sonenshein, A. L., Hoch, J. A., and Losick, R., eds. (1993) Bacillus subtilis, ASM Press: Washington, D.C.).

Promoter transcriptional control is influenced by several mechanisms of repression or activation. Specific regulatory proteins which bind promoters have the ability to block (repressors) or to assist (activators) the binding of the RNA holoenzyme, and thus to regulate transcription. The binding of these repressor and activator molecules in turn is regulated by their interactions with other molecules, such as proteins or other metabolic compounds. Transcription may alternately be regulated by factors influencing processes such as elongation or termination (see, e.g., Sonenshein, A. L., Hoch, J. A., and Losick, R., eds. (1993) Bacillus subtilis, ASM Press: Washington, D.C.). The ability to regulate transcription of genes in response to a variety of environmental or metabolic cues affords cells the ability to tightly control when a gene may be expressed and or how much of a gene product may be present in the cell at one time. This in turn prevents unnecessary expenditure of energy or unnecessary utilization of possibly scarce intermediate compounds or cofactors.

D. Translation and tRNA-Aminoacyl Synthetases

Translation is the process by which a polypeptide is synthesized from amino acids according to the information contained within an mRNA molecule. The main components of this process are ribosomes and specific initiation or elongation factors, such as IF1-3, EF-G, and EFTu (see, e.g., Sonenshein, A. L., Hoch, J. A., Losick, R., eds. (1993) Bacillus subtilis, ASM Press: Washington, D.C.).

Each codon of the mRNA molecule encodes a particular amino acid. The conversion from mRNA to amino acid is effected by transfer RNA (tRNA) molecules. These molecules consist of a single strand of RNA (between 60 and 100 bases), which exists in an L-shaped three dimensional structure having protruding areas, or ‘arms’. One such arm forms base pairs with a particular codon sequence on the mRNA molecule. A second arm interacts specifically with a particular amino acid (the one encoded by the codon). Other arms of the tRNA include the variable arm, the TψC arm (which bears thimidylate and pseudouridylate modifications), and the D arm (which bears a dihydrouridine modification). The function of these latter structures remains unknown, but their conservation between tRNA molecules suggests a role in protein synthesis.

In order for the nucleic acid-based tRNA molecule to associate with the correct amino acid, a family of enzymes, termed the aminoacyl-tRNA synthetases, must act. There exist many different of these enzymes, each of which is specific for a particular tRNA and a particular amino acid. These enzymes link the 3′ hydroxyl of the terminal tRNA adenosine ribose moiety to the amino acid in a two step reaction. First, the enzyme is activated by reaction with ATP and the amino acid to result in an aminoacyl-tRNA synthetase-aminoacyl adenylate complex. Second, the aminoacyl group is transferred from the enzyme to the target tRNA where it remains in the high-energy state. Binding of the tRNA molecule to its cognate codon on the mRNA molecule then brings the high-energy amino acid attached to the tRNA into contact with the ribosome. Within the ribosome, the amino-acid charged tRNA (aminoacyl-tRNA) occupies one binding site (the A site) adjacent to a second site (the P site) containing a tRNA molecule whose amino acid arm is attached to the nascent polypeptide chain (peptidyl-tRNA). The activated amino acid on the aminoacyl-tRNA is sufficiently reactive that a peptide bond spontaneously forms between this amino acid and the next amino acid on the nascent polypeptide chain. Hydrolysis of GTP provides the energy for the transfer of the now-polypeptide chain-loaded tRNA from the A site to the P site of the ribosome, and the process repeats until a stop codon is reached.

There are a number of different steps at which translation may be regulated. These include the binding of the ribosome to mRNA, the presence of mRNA secondary structure, codon usage, or the abundance of particular tRNAs. Also, special regulation mechanisms such as attenuation may act at the level of translation. For an in-depth review of many of these mechanisms, see, e.g., Vellanoweth, R. L. (1993) “Translation and its Regulation” in: Bacillus subtilis and other Gram Positive Bacteria, Sonenshein, A. L. et al., eds., ASM Press: Washington D.C., p. 699-711, and references cited therein.

E. Protein Folding and Secretion

Synthesis of proteins by the ribosome results in polypeptide chains, which must take on a three-dimensional form before the protein can function normally. This three-dimensional structure is achieved by a process of folding. Polypeptide chains are flexible, and (in principle) move readily and freely in solution until they attain a conformation which results in a stable three-dimensional structure. However, it is sometimes difficult for proteins to fold correctly, either due to environmental conditions (e.g., high temperature, where the extra kinetic energy present in the system makes it more difficult for the polypeptide to settle in the energy well of a stable structure) or due to the nature of the protein itself (e.g., the hydrophobic regions in nearby polypeptides have a tendency to aggregate and thereby sequester themselves from aqueous solution).

Proteinaceous factors have been identified that are able to catalyze, chaperone, or otherwise assist in the folding of proteins being synthesized either co- or posttranslationally. Members of these protein folding molecules are the prolyl-peptidyl isomerases (e.g., trigger factor, cyclophilin, and FKBP homologs), and proteins of the heat shock protein group (e.g., DnaK, DnaJ, GroEL, small heat shock proteins, HtpG and members of the Clp family (e.g., ClpA, CIpB, ClpW, ClpP, and ClpX)). Many of these proteins are essential for the viability of cells: in addition to their functions in protein folding, translocation, and processing, they frequently serve as key targets for the overall regulation of protein synthesis (see, e.g., Bukau, B., (1993) Molecular Microbiology 9(4): 671-680; Bukau, B., and Horwich, A. L. (1998) Cell 92(3):351-366; Hesterkamp, T., Bukau, C. (1996) FEBS Lett. 389(1):32-34; Yaron, A., Naider, F. (1993) Critical Reviews in Biochemistry and Molecular Biology 28(1):31-81; Scheibel, R., Buchner, J. (1998) Biochemical Pharmacology 56(6):675-682; Ellis, R. J., Hartl, F. U. (1996) FASEB Journal 10(1): 20-26; Wawrzynow, A. et al (1996) Molecular Microbiology 21(5): 895-899; Ewalt, K. L., et al. (1997) Cell 90(3): 491-500).

Chaperones identified thus far function in one of two ways: they either bind and stabilize polypeptides, or they provide an environment in which folding may occur without interference. The former group, including, e.g., DnaK, DnaJ, and the heat shock proteins, bind directly to the nascent or misfolded polypeptide, frequently with concomitant ATP hydrolysis. The association of the chaperone prevents the polypeptide from aggregating with other polypeptides, and can force such aggregates to dissipate if they have already formed. After interaction with a second chaperone, GrpE (which permits an ADP-ATP exchange to occur), the polypeptide is released in a molten globule state and is permitted to fold. If misfolding occurs, the chaperones again associate with the misfolded protein, forcing it to return to an unfolded state. This cycle may be repeated until the protein is correctly folded. Unlike the first type of chaperones, which simply bind to the polypeptide, the second group (e.g. GroEL/ES) not only bind to the polypeptide, but also completely surround it such that it is protected from the surrounding environment. The GroEL/ES complex is composed of 2 stacked 14-member rings having a hydrophobic interior surface, and a 7-membered ring ‘cap’. The polypeptide is drawn into the channel in the center of this complex in an ATP-dependent reaction where it is able to fold without interference from other polypeptides. Incorrectly folded proteins are not released from the complex.

An important step in protein folding is the creation of disulfide bonds. These bonds, either within a subunit or between subunits of a protein, are critical for protein stability. Disulfide bonds form readily in aqueous solution, and incorrect disulfide bond formation is difficult to reverse without the aid of a reducing environment. To assist in this process of correct disulfide bond formation, thiol-containing molecules, such as glutathione or thioredoxin, and their respective oxidation/reduction systems are found in the cytosol of most cells (Loferer, H., Hennecke, H. (1994) Trends in Biochemical Sciences 19(4): 169-171).

There are times, however, when folding of nascent polypeptide chains is not desirable, such as when these polypeptides are to be secreted. The folding process generally results in the hydrophobic regions of the protein being in the center of the protein, away from aqueous solution, and the hydrophilic regions being presented at the outer surfaces of the protein. This conformational arrangement, while creating greater stability for the protein, makes it difficult for the protein to be translocated across membranes, since the hydrophobic core of the membrane is inherently incompatible with the hydrophilic exterior of the protein. Thus, proteins synthesized by the cell which must be secreted to the exterior of the cell (e.g., cell surface enzymes and membrane receptors) or which must be inserted into the membrane itself (e.g., transporter proteins and channel proteins) are generally secreted or inserted prior to folding. The same chaperones which prevent aggregation of nascent polypeptide chains also prevent folding of polypeptides until they are disengaged. Thus, these proteins may ‘escort’ nascent polypeptide chains to an appropriate cellular location where they either are removed, thereby permitting folding, or they transfer the polypeptide to a transport system which will either secrete the polypeptide or aid its insertion into a membrane.

A specialized protein machinery has evolved that specifically detects, binds, transports, and processes proteins bearing specific prosequences (these sequences are later removed from the protein by cleavage). This machinery consists of a number of proteins which are collectively termed the sec (type II secretion) system (for review, see Gilbert, M. et al. (1995) Critical Reviews in Biotechnology 15(1): 13-39 and references therein; Freudl, R. (1992) Journal of Biotechnology 23(3): 231-240 and references therein; Neidhardt, F. C. et al. (1996) E. coli and Salmonella ASM Press: Washington, D.C., p. 967-978; Binet, R. et al. (1997) Gene 192(1): 7-11; and Rapoport, T. A. (1986) Critical Reviews in Biochemistry 20(1): 73-137, and references therein). The sec system is composed of chaperones (e.g., SecA and SecB), integral membrane proteins, also called translocases (e.g., SecY, SecE, and SecG), and signal peptidases (e.g., LepB). The nascent polypeptide having a prosequence directing secretion is bound by SecB, which delivers it to SecA at the inner surface of the cell membrane. Sec A binds to the prosequence and, upon ATP hydrolysis, inserts into the membrane and forces a portion of the polypeptide through the membrane as well. The remainder of the polypeptide is guided through the membrane by a complex of translocases, such as SecY, SecE, and SecG. Finally, the signal peptidase cleaves off the prosequence and the polypeptide is free on the extracellular side of the membrane, where it spontaneously folds.

Sec-independent secretion mechanisms are also known. For example, the signal recognition particle-dependent pathway involves the binding of a signal recognition particle (SRP) protein to the nascent polypeptide as it is being synthesized, forcing the ribosome to stall. A receptor for SRP at the inner surface of the membrane then binds the ribosome-polypeptide-SRP complex. Hydrolysis of GTP provides the energy necessary to transfer the complex to the sec translocase complex, where the nascent polypeptide is guided across the membrane as it is synthesized by the ribosome. Other secretion mechanisms specific to only a few proteins are also known to exist.

III. Elements and Methods of the Invention

The present invention is based, at least in part, on the discovery of novel molecules, referred to herein as SES nucleic acid and protein molecules, which participate in C. glutamicum DNA repair or recombination, in the transposition or other rearrangement of C. glutamicum DNA, in C. glutamicum gene expression (e.g., the processes of transcription or translation), or in protein folding or protein secretion from this microorganism. In one embodiment, the SES molecules participate in the repair or recombination of DNA, in the transposition of genetic material, in gene expression (i.e., the processes of transcription or translation), in protein folding, or in protein secretion in Corynebacterium glutamicum. In a preferred embodiment, the activity of the SES molecules of the present invention with regard to DNA repair or recombination, transposition of DNA, gene expression, protein folding or protein secretion has an impact on the production of a desired fine chemical by this organism. In a particularly preferred embodiment, the SES molecules of the invention are modulated in activity, such that the C. glutamicum cellular processes in which the SES molecules participate (e.g., DNA repair or recombination, transposition of DNA, gene expression, protein folding, or protein secretion) are also altered in activity, resulting either directly or indirectly in a modulation of the yield, production, and/or efficiency of production of a desired fine chemical by C. glutamicum.

The language, “SES protein” or “SES polypeptide” includes proteins which participate in a number of cellular processes related to C. glutamicum genetic stability, gene expression, protein folding, or protein secretion. For example, an SES protein may be involved in C. glutamicum DNA repair or recombination mechanisms, in rearrangements of C. glutamicum genetic material (such as those mediated by transposons), in transcription or translation of genes in this microorganism, in the mediation of C. glutamicum protein folding (such as the activity of chaperones) or in secretion of proteins from C. glutamicum cells (e.g., the sec system). Examples of SES proteins include those encoded by the SES genes set forth in Table 1 and Appendix A. The terms “SES gene” or “SES nucleic acid sequence” include nucleic acid sequences encoding an SES protein, which consist of a coding region and also corresponding untranslated 5′ and 3′ sequence regions. Examples of SES genes include those set forth in Table 1. The terms “production” or “productivity” are art-recognized and include the concentration of the fermentation product (for example, the desired fine chemical) formed within a given time and a given fermentation volume (e.g., kg product per hour per liter). The term “efficiency of production” includes the time required for a particular level of production to be achieved (for example, how long it takes for the cell to attain a particular rate of output of a fine chemical). The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The terms “degradation” or a “degradation pathway” are art-recognized and include the breakdown of a compound, preferably an organic compound, by a cell to degradation products (generally speaking, smaller or less complex molecules) in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound. The term “DNA repair” is art-recognized and includes cellular mechanisms whereby errors in DNA (due either to damage, such as, but not limited to, ultraviolet radiation, methylases, low-fidelity replication, or mutagens) are excised and corrected. The term “recombination” or “DNA recombination” is art-recognized and includes cellular mechanisms whereby extensive DNA damage affecting both strands of a DNA molecule is corrected by homologous recombination with another, undamaged copy of the DNA molecule within the same cell. Such repairs are generally low-fidelity, and may result in genetic rearrangements. The term “transposon” is art-recognized and includes a DNA element which is able to insert randomly throughout the genome of an organism, and which may result in the disruption of genes or their regulatory regions, or in duplications, inversions, deletions, and other genetic rearrangements. The term “protein folding” is art-recognized and includes the movement of a polypeptide chain through multiple three-dimensional configurations until the stable, active, three-dimensional configuration is attained. The formation of disulfide bonds and the sequestration of hydrophobic regions from the surrounding aqueous solution provide some of the driving forces for this folding process, and correct folding may be enhanced by the activity of chaperones. The terms “secretion” or “protein secretion” is art-recognized and includes the movement of proteins from the interior of the cell to the exterior of the cell, in a mechanism whereby a system of secretion proteins permits their transit across the cellular membrane to the exterior of the cell.

In another embodiment, the SES molecules of the invention are capable of modulating the production of a desired molecule, such as a fine chemical, in a microorganism such as C. glutamicum. There are a number of mechanisms by which the alteration of an SES protein of the invention may directly affect the yield, production, and/or efficiency of production of a fine chemical from a C. glutamicum strain incorporating such an altered protein. For example, modulation of proteins involved directly in transcription or translation (e.g., polymerases or ribosomes) such that they are increased in number or in activity should increase global cellular transcription or translation (or rates of these processes). This increased cellular gene expression should include those proteins involved in fine chemical biosynthesis, so an increase in yield, production, or efficiency of production of one or more desired compounds may occur. Modifications to the transcriptional/translational protein machinery of C. glutamicum such that the regulation of these proteins is altered may also permit increased expression of genes involved in the production of fine chemicals. Modulation of the activity or number of proteins involved in polypeptide folding may permit an increase in the overall production of correctly folded molecules in the cell, thereby increasing the possibility that desired proteins (e.g., fine chemical biosynthetic proteins) are able to function properly. Further, by mutating proteins involved in secretion from C. glutamicum such that they are increased in number or activity, it may be possible to increase the secretion of a fine chemical (e.g., an enzyme) from cells in fermentor culture, where it may be readily recovered.

Genetic modification of the SES molecules of the invention may also result in indirect modulation of production of one or more fine chemicals. For example, by increasing the number or activity of a DNA repair or recombination protein of the invention, one may increase the ability of the cell to detect and repair DNA damage. This should effectively increase the ability of the cell to maintain a mutated gene within its genome, thereby increasing the likelihood that a transgene engineered into C. glutamicum (e.g., encoding a protein which will increase biosynthesis of a fine chemical) will not be lost during culture of the microorganism. Conversely, by decreasing the number or activity of one or more DNA repair or recombination proteins, it may be possible to increase the genetic instability of the organism. Such manipulations should improve the ability of the organism to be modified by mutagenesis without the introduced mutation being corrected. The same holds true for proteins involved in transposition or rearrangement of genetic elements in C. glutamicum (e.g., transposons). By mutagenizing these proteins such that they are either increased or decreased in number or activity, it is possible to simultaneously increase or decrease the genetic stability of the microorganism. This has a profound impact on the ability of any other mutation to be introduced into C. glutamicum, and on the ability of introduced mutations to be retained. Transposons also offer a convenient mechanism by which mutagenesis of C. glutamicum may be performed; duplication of desired genes (e.g., fine chemical biosynthetic genes) is readily accomplished by transposon mutagenesis, as is disruption of undesired genes (e.g., genes encoding proteins involved in degradation of desired fine chemicals).

By modulating one or more proteins (e.g. sigma factors) involved in the regulation of transcription or translation in response to particular environmental conditions, it may be possible to prevent the cell from slowing or stopping protein synthesis under unfavorable environmental conditions, such as those found in large-scale fermentor culture. This should lead to increased gene expression, which in turn may permit increased biosynthesis of desired fine chemicals under such conditions. Many such secreted proteins have functions critical for cell viability (e.g., cell surface proteases or receptors). An alteration of a secretory pathway such that these proteins are more readily transported to their extracellular location may improve the overall viability of the cell, and thus result in greater numbers of C. glutamicum cells capable of producing fine chemicals during large-scale culture. Further, since certain bacterial protein secretion pathways (e.g., the sec system) are known to participate in the insertion of integral membrane proteins (such as receptors, channels, pores, or transporters) into the membrane, the modulation of activity of proteins involved in protein secretion from C. glutamicum may affect the ability of the cell to excrete waste products or to import necessary metabolites. If the activity of these secretory proteins is increased, then the ability of the cell to produce fine chemicals may be similarly increased (due to an increase in the presence of transporters/channels in the membrane which may import nutrients or excrete waste products). If the activity of these proteins is decreased, then there may be insufficient nutrients available to support overproduction of desired compounds, or waste products may interfere with fine chemical biosynthesis.

The isolated nucleic acid sequences of the invention are contained within the genome of a Corynebacterium glutamicum strain available through the American Type Culture Collection, given designation ATCC 13032. The nucleotide sequence of the isolated C. glutamicum SES DNAs and the predicted amino acid sequences of the C. glutamicum SES proteins are shown in Appendices A and B, respectively. Computational analyses were performed which classified and/or identified these nucleotide sequences as sequences which encode proteins involved in the repair or recombination of DNA, in the transposition of genetic material, in gene expression (i.e., the processes of transcription or translation), in protein folding, or in protein secretion in Corynebacterium glutamicum.

The present invention also pertains to proteins which have an amino acid sequence which is substantially homologous to an amino acid sequence of Appendix B. As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The SES protein or a biologically active portion or fragment thereof of the invention can participate in the repair or recombination of DNA, in the transposition of genetic material, in gene expression (i.e., the processes of transcription or translation), in protein folding, or in protein secretion in Corynebacterium glutamicum, or have one or more of the activities set forth in Table 1.

Various aspects of the invention are described in further detail in the following subsections:

A. Isolated Nucleic Acid Molecules

One aspect of the invention pertains to isolated nucleic acid molecules that encode SES polypeptides or biologically active portions thereof, as well as nucleic acid fragments sufficient for use as hybridization probes or primers for the identification or amplification of SES-encoding nucleic acid (e.g., SES DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′ end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated SES nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a C. glutamicum cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a C. glutamicum SES DNA can be isolated from a C. glutamicum library using all or portion of one of the sequences of Appendix A as a hybridization probe and standard hybridization techniques (e.g., as described in Sambrook, J., Fritsh, E. F., and Maniatis, T. Molecular Cloning: A Laboratory Manual. 2nd ed, Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from normal endothelial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an SES nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Corynebacterium glutamicum SES DNAs of the invention. This DNA comprises sequences encoding SES proteins (i.e., the “coding region”, indicated in each sequence in Appendix A), as well as 5′ untranslated sequences and 3′ untranslated sequences, also indicated in Appendix A. Alternatively, the nucleic acid molecule can comprise only the coding region of any of the sequences in Appendix A.

For the purposes of this application, it will be understood that each of the sequences set forth in Appendix A has an identifying RXA, RXN, or RXS number having the designation “RXA”, “RXN” or “RXS” followed by 5 digits (i.e., RXA01278, RXN01559, or RXS00061). Each of these sequences comprises up to three parts: a 5′ upstream region, a coding region, and a downstream region. Each of these three regions is identified by the same RXA, RXN, or RXS designation to eliminate confusion. The recitation “one of the sequences in Appendix A”, then, refers to any of the sequences in Appendix A, which may be distinguished by their differing RXA, RXN, or RXS designations. The coding region of each of these sequences is translated into a corresponding amino acid sequence, which is set forth in Appendix B. The sequences of Appendix B are identified by the same RXA, RXN, or RXS designations as Appendix A, such that they can be readily correlated. For example, the amino acid sequences in Appendix B designated RXA01278, RXN01559, and RXS00061 are translations of the coding regions of the nucleotide sequence of nucleic acid molecules RXA01278, RXN01559, and RXS00061 respectively, in Appendix A. Each of the RXA, RXN, and RXS nucleotide and amino acid sequences of the invention has also been assigned a SEQ ID NO, as indicated in Table 1. For example, as set forth in Table 1, the nucleotide sequence of RXN01559 is SEQ ID NO:5, and the amino acid sequence of RXN01559 is SEQ ID NO:6.

Several of the genes of the invention are “F-designated genes”. An F-designated gene includes those genes set forth in Table 1 which have an ‘F’ in front of the RXA, RXN or RXS designation. For example, SEQ ID NO:7, designated, as indicated on Table 1, as “F RXA00935”, is an F-designated gene, as are SEQ ID NOs: 9, 29, and 37 (designated on Table 1 as “F RXA01559”, “F RXA00484”, and “F RXA01670”, respectively).

In one embodiment, the nucleic acid molecules of the present invention are not intended to include those compiled in Table 2. In the case of the dapD gene, a sequence for this gene was published in Wehrmann, A., et al. (1998) J. Bacteriol. 180(12): 3159-3165. However, the sequence obtained by the inventors of the present application is significantly longer than the published version. It is believed that the published version relied on an incorrect start codon, and thus represents only a fragment of the actual coding region.

In another preferred embodiment, an isolated nucleic acid molecule of the invention comprises a nucleic acid molecule which is a complement of one of the nucleotide sequences shown in Appendix A, or a portion thereof. A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In still another preferred embodiment, an isolated nucleic acid molecule of the invention comprises a nucleotide sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence shown in Appendix A, or a portion thereof. Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an SES protein. The nucleotide sequences determined from the cloning of the SES genes from C. glutamicum allows for the generation of probes and primers designed for use in identifying and/or cloning SES homologues in other cell types and organisms, as well as SES homologues from other Corynebacteria or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone SES homologues. Probes based on the SES nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a diagnostic test kit for identifying cells which misexpress an SES protein, such as by measuring a level of an SES-encoding nucleic acid in a sample of cells, e.g., detecting SES mRNA levels or determining whether a genomic SES gene has been mutated or deleted.

In one embodiment, the nucleic acid molecule of the invention encodes a protein or portion thereof which includes an amino acid sequence which is sufficiently homologous to an amino acid sequence of Appendix B such that the protein or portion thereof maintains the ability to participate in the repair or recombination of DNA, in the transposition of genetic material, in gene expression (i.e., the processes of transcription or translation), in protein folding, or in protein secretion in Corynebacterium glutamicum. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B) amino acid residues to an amino acid sequence of Appendix B such that the protein or portion thereof is able to participate in the repair or recombination of DNA, in the transposition of genetic material, in gene expression (i.e., the processes of transcription or translation), in protein folding, or in protein secretion in Corynebacterium glutamicum. Proteins involved in C. glutamicum genetic stability, gene expression, protein folding or protein secretion, as described herein, may play a role in the production and secretion of one or more fine chemicals. Examples of such activities are also described herein. Thus, “the function of an SES protein” contributes either directly or indirectly to the yield, production, and/or efficiency of production of one or more fine chemicals. Examples of SES protein activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B.

Portions of proteins encoded by the SES nucleic acid molecules of the invention are preferably biologically active portions of one of the SES proteins. As used herein, the term “biologically active portion of an SES protein” is intended to include a portion, e.g., a domain/motif, of an SES protein that participate in the repair or recombination of DNA, in the transposition of genetic material, in gene expression (i.e., the processes of transcription or translation), in protein folding, or in protein secretion in Corynebacterium glutamicum, or has an activity as set forth in Table 1. To determine whether an SES protein or a biologically active portion thereof can participate in the repair or recombination of DNA, in the transposition of genetic material, in gene expression (i.e., the processes of transcription or translation), in protein folding, or in protein secretion in Corynebacterium glutamicum, an assay of enzymatic activity may be performed. Such assay methods are well known to those of ordinary skill in the art, as detailed in Example 8 of the Exemplification.

Additional nucleic acid fragments encoding biologically active portions of an SES protein can be prepared by isolating a portion of one of the sequences in Appendix B, expressing the encoded portion of the SES protein or peptide (e.g., by recombinant expression in vitro) and assessing the activity of the encoded portion of the SES protein or peptide.

The invention further encompasses nucleic acid molecules that differ from one of the nucleotide sequences shown in Appendix A (and portions thereof) due to degeneracy of the genetic code and thus encode the same SES protein as that encoded by the nucleotide sequences shown in Appendix A. In another embodiment, an isolated nucleic acid molecule of the invention has a nucleotide sequence encoding a protein having an amino acid sequence shown in Appendix B. In a still further embodiment, the nucleic acid molecule of the invention encodes a full length C. glutamicum protein which is substantially homologous to an amino acid sequence of Appendix B (encoded by an open reading frame shown in Appendix A).

It will be understood by one of ordinary skill in the art that in one embodiment the sequences of the invention are not meant to include the sequences of the prior art, such as those Genbank sequences set forth in Tables 2 or 4 which were available prior to the present invention. In one embodiment, the invention includes nucleotide and amino acid sequences having a percent identity to a nucleotide or amino acid sequence of the invention which is greater than that of a sequence of the prior art (e.g., a Genbank sequence (or the protein encoded by such a sequence) set forth in Tables 2 or 4). For example, the invention includes a nucleotide sequence which is greater than and/or at least 71% identical to the nucleotide sequence designated RXA01278 (SEQ ID NO: 1), a nucleotide sequence which is greater than and/or at least 38% identical to the nucleotide sequence designated RXA01020 (SEQ ID NO:25), and a nucleotide sequence which is greater than and/or at least 54% identical to the nucleotide sequence designated RXA02078 (SEQ ID NO:39). One of ordinary skill in the art would be able to calculate the lower threshold of percent identity for any given sequence of the invention by examining the GAP-calculated percent identity scores set forth in Table 4 for each of the three top hits for the given sequence, and by subtracting the highest GAP-calculated percent identity from 100 percent. One of ordinary skill in the art will also appreciate that nucleic acid and amino acid sequences having percent identities greater than the lower threshold so calculated (e.g., at least 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the C. glutamicum SES nucleotide sequences shown in Appendix A, it will be appreciated by those of ordinary skill in the art that DNA sequence polymorphisms that lead to changes in the amino acid sequences of SES proteins may exist within a population (e.g., the C. glutamicum population). Such genetic polymorphism in the SES gene may exist among individuals within a population due to natural variation. As used herein, the terms “gene” and “recombinant gene” refer to nucleic acid molecules comprising an open reading frame encoding an SES protein, preferably a C. glutamicum SES protein. Such natural variations can typically result in 1-5% variance in the nucleotide sequence of the SES gene. Any and all such nucleotide variations and resulting amino acid polymorphisms in SES that are the result of natural variation and that do not alter the functional activity of SES proteins are intended to be within the scope of the invention.

Nucleic acid molecules corresponding to natural variants and non-C. glutamicum homologues of the C. glutamicum SES DNA of the invention can be isolated based on their homology to the C. glutamicum SES nucleic acid disclosed herein using the C. glutamicum DNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural C. glutamicum SES protein.

In addition to naturally-occurring variants of the SES sequence that may exist in the population, one of ordinary skill in the art will further appreciate that changes can be introduced by mutation into a nucleotide sequence of Appendix A, thereby leading to changes in the amino acid sequence of the encoded SES protein, without altering the functional ability of the SES protein. For example, nucleotide substitutions leading to amino acid substitutions at “non-essential” amino acid residues can be made in a sequence of Appendix A. A “non-essential” amino acid residue is a residue that can be altered from the wild-type sequence of one of the SES proteins (Appendix B) without altering the activity of said SES protein, whereas an “essential” amino acid residue is required for SES protein activity. Other amino acid residues, however, (e.g., those that are not conserved or only semi-conserved in the domain having SES activity) may not be essential for activity and thus are likely to be amenable to alteration without altering SES activity.

Accordingly, another aspect of the invention pertains to nucleic acid molecules encoding SES proteins that contain changes in amino acid residues that are not essential for SES activity. Such SES proteins differ in amino acid sequence from a sequence contained in Appendix B yet retain at least one of the SES activities described herein. In one embodiment, the isolated nucleic acid molecule comprises a nucleotide sequence encoding a protein, wherein the protein comprises an amino acid sequence at least about 50% homologous to an amino acid sequence of Appendix B and is capable of participating in the repair or recombination of DNA, in the transposition of genetic material, in gene expression (i.e., the processes of transcription or translation), in protein folding, or in protein secretion in Corynebacterium glutamicum, or has one or more activities set forth in Table 1. Preferably, the protein encoded by the nucleic acid molecule is at least about 50-60% homologous to one of the sequences in Appendix B, more preferably at least about 60-70% homologous to one of the sequences in Appendix B, even more preferably at least about 70-80%, 80-90%, 90-95% homologous to one of the sequences in Appendix B, and most preferably at least about 96%, 97%, 98%, or 99% homologous to one of the sequences in Appendix B.

To determine the percent homology of two amino acid sequences (e.g., one of the sequences of Appendix B and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions ×100).

An isolated nucleic acid molecule encoding an SES protein homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an SES protein is preferably replaced with another amino acid residue from the same side chain family. Alternatively, in another embodiment, mutations can be introduced randomly along all or part of an SES coding sequence, such as by saturation mutagenesis, and the resultant mutants can be screened for an SES activity described herein to identify mutants that retain SES activity. Following mutagenesis of one of the sequences of Appendix A, the encoded protein can be expressed recombinantly and the activity of the protein can be determined using, for example, assays described herein (see Example 8 of the Exemplification).

In addition to the nucleic acid molecules encoding SES proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded DNA molecule or complementary to an mRNA sequence. Accordingly, an antisense nucleic acid can hydrogen bond to a sense nucleic acid. The antisense nucleic acid can be complementary to an entire SES coding strand, or to only a portion thereof. In one embodiment, an antisense nucleic acid molecule is antisense to a “coding region” of the coding strand of a nucleotide sequence encoding an SES protein. The term “coding region” refers to the region of the nucleotide sequence comprising codons which are translated into amino acid residues (e.g., the entire coding region of SEQ ID NO. 1 (RXA01278) comprises nucleotides 1 to 2127). In another embodiment, the antisense nucleic acid molecule is antisense to a “noncoding region” of the coding strand of a nucleotide sequence encoding SES. The term “noncoding region” refers to 5′ and 3′ sequences which flank the coding region that are not translated into amino acids (i.e., also referred to as 5′ and 3′ untranslated regions).

Given the coding strand sequences encoding SES disclosed herein (e.g., the sequences set forth in Appendix A), antisense nucleic acids of the invention can be designed according to the rules of Watson and Crick base pairing. The antisense nucleic acid molecule can be complementary to the entire coding region of SES mRNA, but more preferably is an oligonucleotide which is antisense to only a portion of the coding or noncoding region of SES mRNA. For example, the antisense oligonucleotide can be complementary to the region surrounding the translation start site of SES mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an SES protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′—O— methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave SES mRNA transcripts to thereby inhibit translation of SES mRNA. A ribozyme having specificity for an SES-encoding nucleic acid can be designed based upon the nucleotide sequence of an SES DNA disclosed herein (i.e., SEQ ID NO. 1 (RXA01278 in Appendix A)). For example, a derivative of a Tetrahymena L-19 IVS RNA can be constructed in which the nucleotide sequence of the active site is complementary to the nucleotide sequence to be cleaved in an SES-encoding mRNA. See, e.g., Cech et al. U.S. Pat. No. 4,987,071 and Cech et al. U.S. Pat. No. 5,116,742. Alternatively, SES mRNA can be used to select a catalytic RNA having a specific ribonuclease activity from a pool of RNA molecules. See, e.g., Bartel, D. and Szostak, J. W. (1993) Science 261:1411-1418.

Alternatively, SES gene expression can be inhibited by targeting nucleotide sequences complementary to the regulatory region of an SES nucleotide sequence (e.g., an SES promoter and/or enhancers) to form triple helical structures that prevent transcription of an SES gene in target cells. See generally, Helene, C. (1991) Anticancer Drug Des. 6(6):569-84; Helene, C. et al. (1992) Ann. NY. Acad. Sci. 660:27-36; and Maher, L. J. (1992) Bioassays 14(12):807-15.

B. Recombinant Expression Vectors and Host Cells

Another aspect of the invention pertains to vectors, preferably expression vectors, containing a nucleic acid encoding an SES protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which are operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, lacI^{q}-, T7-, T5-, T3-, gal-, trc-, ara-, SP6-, arny, SPO2, λP_{R}- or λP_{L}-, which are used preferably in bacteria. Additional regulatory sequences are, for example, promoters from yeasts and fungi, such as ADC1, MFα, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., SES proteins, mutant forms of SES proteins, fusion proteins, etc.).

The recombinant expression vectors of the invention can be designed for expression of SES proteins in prokaryotic or eukaryotic cells. For example, SES genes can be expressed in bacterial cells such as C. glutamicum, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens-mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the SES protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant SES protein unfused to GST can be recovered by cleavage of the fusion protein with thrombin.

Examples of suitable inducible non-fusion E. coli expression vectors include pTrc (Amann et al., (1988) Gene 69:301-315) pLG338, pACYC184, pBR322, pUC18, pUC19, pKC30, pRep4, pHS1, pHS2, pPLc236, pMBL24, pLG200, pUR290, pIN-III113-B1, λgt 11, pBdC1, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier: N.Y. IBSN 0 444 904018). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194, or pBD214 are suited for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: N.Y. IBSN 0 444 904018). One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20: 2111-2118). Such alteration of nucleic acid sequences of the invention can be carried out by standard DNA synthesis techniques.

In another embodiment, the SES protein expression vector is a yeast expression vector. Examples of vectors for expression in yeast S cerevisiae include pYepSec1 (Baldari, et al., (1987) Embo J 6:229-234, 2μ, pAG-1, Yep6, Yep13, pEMBLYe23, pMFa (Kurjan and Herskowitz, (1982) Cell 30:933-943), pJRY88 (Schultz. et al., (1987) Gene 54:113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: N.Y. (IBSN 0 444 904018).

Alternatively, the SES proteins of the invention can be expressed in insect cells using baculovirus expression vectors. Baculovirus vectors available for expression of proteins in cultured insect cells (e.g., Sf9 cells) include the pAc series (Smith et al (1983) Mol. Cell. Biol. 3:2156-2165) and the pVL series (Lucklow and Summers (1989) Virology 170:31-39).

In another embodiment, the SES proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid Res. 12: 8711-8721, and include pLGV23, pGHlac+, pBIN19, pAK2004, and pDH51 (Pouwels et al., eds. (1985) Cloning Vectors. Elsevier: N.Y. IBSN 0 444 904018).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to SES mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) (1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an SES protein can be expressed in bacterial cells such as C. glutamicum, insect cells, yeast or mammalian cells (such as Chinese hamster ovary cells (CHO) or COS cells). Other suitable host cells are known to one of ordinary skill in the art. Microorganisms related to Corynebacterium glutamicum which may be conveniently used as host cells for the nucleic acid and protein molecules of the invention are set forth in Table 3.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an SES protein or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an SES gene into which a deletion, addition or substitution has been introduced to thereby alter, e.g., functionally disrupt, the SES gene. Preferably, this SES gene is a Corynebacterium glutamicum SES gene, but it can be a homologue from a related bacterium or even from a mammalian, yeast, or insect source. In a preferred embodiment, the vector is designed such that, upon homologous recombination, the endogenous SES gene is functionally disrupted (i.e., no longer encodes a functional protein; also referred to as a “knock out” vector). Alternatively, the vector can be designed such that, upon homologous recombination, the endogenous SES gene is mutated or otherwise altered but still encodes functional protein (e.g., the upstream regulatory region can be altered to thereby alter the expression of the endogenous SES protein). In the homologous recombination vector, the altered portion of the SES gene is flanked at its 5′ and 3′ ends by additional nucleic acid of the SES gene to allow for homologous recombination to occur between the exogenous SES gene carried by the vector and an endogenous SES gene in a microorganism. The additional flanking SES nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced SES gene has homologously recombined with the endogenous SES gene are selected, using art-known techniques.

In another embodiment, recombinant microorganisms can be produced which contain selected systems which allow for regulated expression of the introduced gene. For example, inclusion of an SES gene on a vector placing it under control of the lac operon permits expression of the SES gene only in the presence of IPTG. Such regulatory systems are well known in the art.

In another embodiment, an endogenous SES gene in a host cell is disrupted (e.g., by homologous recombination or other genetic means known in the art) such that expression of its protein product does not occur. In another embodiment, an endogenous or introduced SES gene in a host cell has been altered by one or more point mutations, deletions, or inversions, but still encodes a functional SES protein. In still another embodiment, one or more of the regulatory regions (e.g., a promoter, repressor, or inducer) of an SES gene in a microorganism has been altered (e.g., by deletion, truncation, inversion, or point mutation) such that the expression of the SES gene is modulated. One of ordinary skill in the art will appreciate that host cells containing more than one of the described SES gene and protein modifications may be readily produced using the methods of the invention, and are meant to be included in the present invention.

A host cell of the invention, such as a prokaryotic or eukaryotic host cell in culture, can be used to produce (i.e., express) an SES protein. Accordingly, the invention further provides methods for producing SES proteins using the host cells of the invention. In one embodiment, the method comprises culturing the host cell of invention (into which a recombinant expression vector encoding an SES protein has been introduced, or into which genome has been introduced a gene encoding a wild-type or altered SES protein) in a suitable medium until SES protein is produced. In another embodiment, the method further comprises isolating SES proteins from the medium or the host cell.

C. Isolated SES Proteins

Another aspect of the invention pertains to isolated SES proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of SES protein in which the protein is separated from cellular components of the cells in which it is naturally or recombinantly produced. In one embodiment, the language “substantially free of cellular material” includes preparations of SES protein having less than about 30% (by dry weight) of non-SES protein (also referred to herein as a “contaminating protein”), more preferably less than about 20% of non-SES protein, still more preferably less than about 10% of non-SES protein, and most preferably less than about 5% non-SES protein. When the SES protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of SES protein in which the protein is separated from chemical precursors or other chemicals which are involved in the synthesis of the protein. In one embodiment, the language “substantially free of chemical precursors or other chemicals” includes preparations of SES protein having less than about 30% (by dry weight) of chemical precursors or non-SES chemicals, more preferably less than about 20% chemical precursors or non-SES chemicals, still more preferably less than about 10% chemical precursors or non-SES chemicals, and most preferably less than about 5% chemical precursors or non-SES chemicals. In preferred embodiments, isolated proteins or biologically active portions thereof lack contaminating proteins from the same organism from which the SES protein is derived. Typically, such proteins are produced by recombinant expression of, for example, a C. glutamicum SES protein in a microorganism such as C. glutamicum.

An isolated SES protein or a portion thereof of the invention can participate in the repair or recombination of DNA, in the transposition of genetic material, in gene expression (i.e., the processes of transcription or translation), in protein folding, or in protein secretion in Corynebacterium glutamicum, or has one or more of the activities set forth in Table 1. In preferred embodiments, the protein or portion thereof comprises an amino acid sequence which is sufficiently homologous to an amino acid sequence of Appendix B such that the protein or portion thereof maintains the ability to participate in the repair or recombination of DNA, in the transposition of genetic material, in gene expression (i.e., the processes of transcription or translation), in protein folding, or in protein secretion in Corynebacterium glutamicum. The portion of the protein is preferably a biologically active portion as described herein. In another preferred embodiment, an SES protein of the invention has an amino acid sequence shown in Appendix B. In yet another preferred embodiment, the SES protein has an amino acid sequence which is encoded by a nucleotide sequence which hybridizes, e.g., hybridizes under stringent conditions, to a nucleotide sequence of Appendix A. In still another preferred embodiment, the SES protein has an amino acid sequence which is encoded by a nucleotide sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of Appendix A, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred SES proteins of the present invention also preferably possess at least one of the SES activities described herein. For example, a preferred SES protein of the present invention includes an amino acid sequence encoded by a nucleotide sequence which hybridizes, e.g., hybridizes under stringent conditions, to a nucleotide sequence of Appendix A, and which can participate in the repair or recombination of DNA, in the transposition of genetic material, in gene expression (i.e., the processes of transcription or translation), in protein folding, or in protein secretion in Corynebacterium glutamicum, or which has one or more of the activities set forth in Table 1.

In other embodiments, the SES protein is substantially homologous to an amino acid sequence of Appendix B and retains the functional activity of the protein of one of the sequences of Appendix B yet differs in amino acid sequence due to natural variation or mutagenesis, as described in detail in subsection I above. Accordingly, in another embodiment, the SES protein is a protein which comprises an amino acid sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to an entire amino acid sequence of Appendix B and which has at least one of the SES activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length C. glutamicum protein which is substantially homologous to an entire amino acid sequence of Appendix B.

Biologically active portions of an SES protein include peptides comprising amino acid sequences derived from the amino acid sequence of an SES protein, e.g., the an amino acid sequence shown in Appendix B or the amino acid sequence of a protein homologous to an SES protein, which include fewer amino acids than a full length SES protein or the full length protein which is homologous to an SES protein, and exhibit at least one activity of an SES protein. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an SES protein. Moreover, other biologically active portions, in which other regions of the protein are deleted, can be prepared by recombinant techniques and evaluated for one or more of the activities described herein. Preferably, the biologically active portions of an SES protein include one or more selected domains/motifs or portions thereof having biological activity.

SES proteins are preferably produced by recombinant DNA techniques. For example, a nucleic acid molecule encoding the protein is cloned into an expression vector (as described above), the expression vector is introduced into a host cell (as described above) and the SES protein is expressed in the host cell. The SES protein can then be isolated from the cells by an appropriate purification scheme using standard protein purification techniques. Alternative to recombinant expression, an SES protein, polypeptide, or peptide can be synthesized chemically using standard peptide synthesis techniques. Moreover, native SES protein can be isolated from cells (e.g., endothelial cells), for example using an anti-SES antibody, which can be produced by standard techniques utilizing an SES protein or fragment thereof of this invention.

The invention also provides SES chimeric or fusion proteins. As used herein, an SES “chimeric protein” or “fusion protein” comprises an SES polypeptide operatively linked to a non-SES polypeptide. An “SES polypeptide” refers to a polypeptide having an amino acid sequence corresponding to an SES protein, whereas a “non-SES polypeptide” refers to a polypeptide having an amino acid sequence corresponding to a protein which is not substantially homologous to the SES protein, e.g., a protein which is different from the SES protein and which is derived from the same or a different organism. Within the fusion protein, the term “operatively linked” is intended to indicate that the SES polypeptide and the non-SES polypeptide are fused in-frame to each other. The non-SES polypeptide can be fused to the N-terminus or C-terminus of the SES polypeptide. For example, in one embodiment the fusion protein is a GST-SES fusion protein in which the SES sequences are fused to the C-terminus of the GST sequences. Such fusion proteins can facilitate the purification of recombinant SES proteins. In another embodiment, the fusion protein is an SES protein containing a heterologous signal sequence at its N-terminus. In certain host cells (e.g., mammalian host cells), expression and/or secretion of an SES protein can be increased through use of a heterologous signal sequence.

Preferably, an SES chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An SES-encoding nucleic acid can be cloned into such an expression vector such that the fusion moiety is linked in-frame to the SES protein.

Homologues of the SES protein can be generated by mutagenesis, e.g., discrete point mutation or truncation of the SES protein. As used herein, the term “homologue” refers to a variant form of the SES protein which acts as an agonist or antagonist of the activity of the SES protein. An agonist of the SES protein can retain substantially the same, or a subset, of the biological activities of the SES protein. An antagonist of the SES protein can inhibit one or more of the activities of the naturally occurring form of the SES protein, by, for example, competitively binding to a downstream or upstream member of a biochemical cascade which includes the SES protein, by binding to a target molecule with which the SES protein interacts, such that no function interaction is possible, or by binding directly to the SES protein and inhibiting its normal activity.

In an alternative embodiment, homologues of the SES protein can be identified by screening combinatorial libraries of mutants, e.g., truncation mutants, of the SES protein for SES protein agonist or antagonist activity. In one embodiment, a variegated library of SES variants is generated by combinatorial mutagenesis at the nucleic acid level and is encoded by a variegated gene library. A variegated library of SES variants can be produced by, for example, enzymatically ligating a mixture of synthetic oligonucleotides into gene sequences such that a degenerate set of potential SES sequences is expressible as individual polypeptides, or alternatively, as a set of larger fusion proteins (e.g., for phage display) containing the set of SES sequences therein. There are a variety of methods which can be used to produce libraries of potential SES homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential SES sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the SES protein coding can be used to generate a variegated population of SES fragments for screening and subsequent selection of homologues of an SES protein. In one embodiment, a library of coding sequence fragments can be generated by treating a double stranded PCR fragment of an SES coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the SES protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of SES homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify SES homologues (Arkin and Yourvan (1992) PNAS 89:7811-7815; Delgrave et al. (1993) Protein Engineering 6(3):327-331).

In another embodiment, cell based assays can be exploited to analyze a variegated SES library, using methods well known in the art.

D. Uses and Methods of the Invention

The nucleic acid molecules, proteins, protein homologues, fusion proteins, primers, vectors, and host cells described herein can be used in one or more of the following methods: identification of C. glutamicum and related organisms; mapping of genomes of organisms related to C. glutamicum; identification and localization of C. glutamicum sequences of interest; evolutionary studies; determination of SES protein regions required for function; modulation of an SES protein activity; and modulation of cellular production of a desired compound, such as a fine chemical.

The SES nucleic acid molecules of the invention have a variety of uses. First, they may be used to identify an organism as being Corynebacterium glutamicum or a close relative thereof. Also, they may be used to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present.

Although Corynebacterium glutamicum itself is nonpathogenic, it is related to pathogenic species, such as Corynebacterium diphtheriae. Corynebacterium diphtheriae is the causative agent of diphtheria, a rapidly developing, acute, febrile infection which involves both local and systemic pathology. In this disease, a local lesion develops in the upper respiratory tract and involves necrotic injury to epithelial cells; the bacilli secrete toxin which is disseminated through this lesion to distal susceptible tissues of the body. Degenerative changes brought about by the inhibition of protein synthesis in these tissues, which include heart, muscle, peripheral nerves, adrenals, kidneys, liver and spleen, result in the systemic pathology of the disease. Diphtheria continues to have high incidence in many parts of the world, including Africa, Asia, Eastern Europe and the independent states of the former Soviet Union. An ongoing epidemic of diphtheria in the latter two regions has resulted in at least 5,000 deaths since 1990.

In one embodiment, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject. C. glutamicum and C. diphtheriae are related bacteria, and many of the nucleic acid and protein molecules in C. glutamicum are homologous to C. diphtheriae nucleic acid and protein molecules, and can therefore be used to detect C. diphtheriae in a subject.

The nucleic acid and protein molecules of the invention may also serve as markers for specific regions of the genome. This has utility not only in the mapping of the genome, but also for functional studies of C. glutamicum proteins. For example, to identify the region of the genome to which a particular C. glutamicum DNA-binding protein binds, the C. glutamicum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of C. glutamicum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as Brevibacterium lactofermentum.

The SES nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the SES nucleic acid molecules of the invention may result in the production of SES proteins having functional differences from the wild-type SES proteins. These proteins may be improved in efficiency or activity, may be present in greater numbers in the cell than is usual, or may be decreased in efficiency or activity.

The invention provides methods for screening molecules which modulate the activity of an SES protein, either by interacting with the protein itself or a substrate or binding partner of the SES protein, or by modulating the transcription or translation of an SES nucleic acid molecule of the invention. In such methods, a microorganism expressing one or more SES proteins of the invention is contacted with one or more test compounds, and the effect of each test compound on the activity or level of expression of the SES protein is assessed.

The modulation of activity of proteins involved in C. glutamicum DNA repair, recombination, or transposition should impact the genetic stability of the cell. For example, by decreasing the number or activity of proteins involved in DNA repair mechanisms, one may decrease the ability of the cell to correct genetic errors, which should permit the simplified introduction of desired mutations into the genome (such as those encoding proteins involved in fine chemical production). Increasing the activity or number of transposons should result in a similarly increased mutation rate in the genome, and can permit facile duplication of desired genes (e.g., those encoding fine chemical biosynthetic proteins) or disruption of undesired genes (e.g., those encoding fine chemical degradation proteins). Conversely, by decreasing the number or activity of transposons or by increasing the number or activity of DNA repair proteins, it may be possible to increase the genetic stability of C. glutamicum, which in turn should result in better retention of introduced mutations in this microorganism through multiple generations in culture. Ideally, during mutagenesis and strain construction, one or more DNA repair systems would be decreased in activity and one or more transposons may be increased in activity, but once the desired mutation had been achieved in a strain, these the reverse would occur. Such manipulation is possible by placement of one or more DNA repair genes or transposons under control of an inducible repressor.

Modulation of proteins involved in transcription and translation in C. glutamicum can have both direct and indirect effects on the production of a fine chemical from these microorganisms. For example, by manipulating a protein which directly translates a gene (e.g., a polymerase) or which directly regulates transcription (e.g., a repressor or activator protein), it is possible to directly affect the expression of the target gene. In the case of genes encoding a protein involved in the biosynthesis or degradation of a fine chemical, this type of genetic manipulation should have a direct effect on the production of this fine chemical. Mutagenesis of a repressor protein such that it can no longer repress its target gene, or mutagenesis of an activator protein such that it is optimized in activity should lead to an increase in transcription of the target gene. If the target gene is, for example, a fine chemical biosynthetic gene, then an increase in production of that chemical may result, due to the overall greater number of transcripts present for the gene, which should result in greater numbers of the protein as well. Increasing the number or activity of a repressor protein for a target sequence or decreasing the number or activity of an activator protein for a target sequence when this sequence is, for example, a fine chemical degradative protein, then a similar increase in production of the fine chemical should result.

Indirect effects on fine chemical production may also arise due to manipulation of proteins involved in transcription and translation. For example, by modulating the activity or number of transcription factors (e.g., the sigma factors) or translational repressors/activators which globally regulate transcription in C. glutamicum in response to environmental or metabolic factors, it should be possible to uncouple cellular transcription from environmental or metabolic regulation. In turn, this may permit continued transcription under conditions which would normally slow or altogether stop gene expression, such as those unfavorable conditions (e.g., high temperature, low oxygen, high waste product levels) which exist in large-scale fermentor cultures. By increasing the rate of gene (e.g., fine chemical biosynthetic gene) expression in such situations, the overall rate of fine product production may also be increased, at least due to the relatively greater number of fine chemical biosynthetic proteins in the cell. Principles and examples for modification of transcription and transcriptional regulation are described in, e.g., Lewin, B. (1990) Genes IV, Part 3: “Controlling procaryotic genes by transcription” Oxford Univ. Press: Oxford, p. 213-301.

Modulation of the activity or number of proteins involved in polypeptide folding (e.g., chaperones) may permit an increase in the overall production of correctly folded molecules in the cell. This has two effects: first, an overall increase in the number of proteins in the cell, due to the fact that fewer proteins are misfolded and degraded, and second, an increase in the number of any given protein that is correctly folded and thus active (see, e.g., Thomas, J. G., Baneyx, F. (1997) Protein Expression and Purification 11(3): 289-296; Luo, Z. H., and Hua, Z. C. (1998) Biochemistry and Molecular Biology International 46(3): 471-477; Dale, G. E., et al. (1994) Protein Engineering 7(7): 925-931; Amrein, K. E. et al. (1995) Proc. Natl. Acad. Sci. U.S. A. 92(4): 1048-1052; and Caspers, P. et al. (1994) Cell. Mol. Biol. 40(5): 635-644). While such mutations result in an increase in the number of active proteins of all kinds, when coupled with additional mutations increasing the activity or number of, e.g., a fine chemical biosynthetic protein, an additive effect in the amount of correctly folded, active desired protein may be obtained.

Manipulation of proteins involved in secretion of polypeptides from C. glutamicum such that they are improved in activity or number may directly improve the secretion of a proteinaceous fine chemical (e.g., an enzyme) from this microorganism. It is significantly easier to harvest and purify fine chemicals when they are secreted into the medium of large-scale cultures than when they are retained in the cell, so the yield and production of a fine chemical should be increased through such secretion system engineering. Genetic manipulation of these secretion proteins may also result in indirect improvements in the production of one or more fine chemicals. First, increased or decreased activity of one or more C. glutamicum secretion systems (as brought about by mutagenesis of one or more SES proteins involved in such pathways) may result in increased or decreased global secretion rates from the cell. Many such secreted proteins have functions critical for cell viability (e.g., cell surface proteases or receptors). An alteration of a secretory pathway such that these proteins are more readily transported to their extracellular location may improve the overall viability of the cell, and thus result in greater numbers of C. glutamicum cells capable of producing fine chemicals during large-scale culture. Second, certain bacterial secretion systems, (e.g., the sec system) are known to play a significant role in the process by which integral membrane proteins (e.g. channels, pores, or transporters) insert into cellular membranes. If the activity of one or more secretory pathway proteins is increased, then the ability of the cell to produce fine chemicals may be similarly increased, due to the presence of increased intracellular nutrient levels or decreased intracellular waste levels. If the activity of one or more such secretory pathway protein is decreased, then there may be insufficient nutrients available to support overproduction of desired compounds, or waste products may interfere with the biosynthesis of desired fine chemicals.

The aforementioned mutagenesis strategies for SES proteins to result in increased yields of a fine chemical from C. glutamicum are not meant to be limiting; variations on these strategies will be readily apparent to one of ordinary skill in the art. Using such strategies, and incorporating the mechanisms disclosed herein, the nucleic acid and protein molecules of the invention may be utilized to generate C. glutamicum or related strains of bacteria expressing mutated SES nucleic acid and protein molecules such that the yield, production, and/or efficiency of production of a desired compound is improved. This desired compound may be any product produced by C. glutamicum, which includes the final products of biosynthesis pathways and intermediates of naturally-occurring metabolic pathways, as well as molecules which do not naturally occur in the metabolism of C. glutamicum, but which are produced by a C. glutamicum strain of the invention.

This invention is further illustrated by the following examples which should not be construed as limiting. The contents of all references, patent applications, patents, published patent applications, Tables, Appendices, and the sequence listing cited throughout this application are hereby incorporated by reference.

### EXEMPLIFICATION

### Example 1

### Preparation of total genomic DNA of Corynebacterium glutamicum ATCC 13032

A culture of Corynebacterium glutamicum (ATCC 13032) was grown overnight at 30° C. with vigorous shaking in BHI medium (Difco). The cells were harvested by centrifugation, the supernatant was discarded and the cells were resuspended in 5 ml buffer-I (5% of the original volume of the culture—all indicated volumes have been calculated for 100 ml of culture volume). Composition of buffer-I: 140.34 g/l sucrose, 2.46 g/l MgSO₄×7H₂O, 10 ml/l KH₂PO₄ solution (100 g/l, adjusted to pH 6.7 with KOH), 50 ml/l M12 concentrate (10 g/l (NH₄)₂SO₄, 1 g/l NaCl, 2 g/l MgSO₄×7H₂O, 0.2 g/l CaCl₂, 0.5 g/l yeast extract (Difco), 10 ml/l trace-elements-mix (200 mg/l FeSO₄×H₂O, 10 mg/l ZnSO₄×7H₂O, 3 mg/l MnCl₂×4H₂O, 30 mg/l H₃BO₃ 20 mg/l CoCl₂×6H₂O, 1 mg/l NiCl₂×6H₂O, 3 mg/l Na₂MoO₄×2H₂O, 500 mg/l complexing agent (EDTA or critic acid), 100 ml/l vitamins-mix (0.2 mg/l biotin, 0.2 mg/l folic acid, 20 mg/l p-amino benzoic acid, 20 mg/l riboflavin, 40 mg/l ca-panthothenate, 140 mg/l nicotinic acid, 40 mg/l pyridoxole hydrochloride, 200 mg/l myo-inositol). Lysozyme was added to the suspension to a final concentration of 2.5 mg/ml. After an approximately 4 h incubation at 37° C., the cell wall was degraded and the resulting protoplasts are harvested by centrifugation. The pellet was washed once with 5 ml buffer-I and once with 5 ml TE-buffer (10 mM Tris-HCl, 1 mM EDTA, pH 8). The pellet was resuspended in 4 ml TE-buffer and 0.5 ml SDS solution (10%) and 0.5 ml NaCl solution (5 M) are added. After adding of proteinase K to a final concentration of 200 μg/ml, the suspension is incubated for ca. 18 h at 37° C. The DNA was purified by extraction with phenol, phenol-chloroform-isoamylalcohol and chloroform-isoamylalcohol using standard procedures. Then, the DNA was precipitated by adding 1/50 volume of 3 M sodium acetate and 2 volumes of ethanol, followed by a 30 min incubation at −20° C. and a 30 min centrifugation at 12,000 rpm in a high speed centrifuge using a SS34 rotor (Sorvall). The DNA was dissolved in 1 ml TE-buffer containing 20 μg/ml RNaseA and dialysed at 4° C. against 1000 ml TE-buffer for at least 3 hours. During this time, the buffer was exchanged 3 times. To aliquots of 0.4 ml of the dialysed DNA solution, 0.4 ml of 2 M LiCl and 0.8 ml of ethanol are added. After a 30 min incubation at −20° C., the DNA was collected by centrifugation (13,000 rpm, Biofuge Fresco, Heraeus, Hanau, Germany). The DNA pellet was dissolved in TE-buffer. DNA prepared by this procedure could be used for all purposes, including southern blotting or construction of genomic libraries.

### Example 2

### Construction of Genomic Libraries in Escherichia Coli of Corynebacterium Glutamicum ATCC13032

Using DNA prepared as described in Example 1, cosmid and plasmid libraries were constructed according to known and well established methods (see e.g., Sambrook, J. et al. (1989) “Molecular Cloning: A Laboratory Manual”, Cold Spring Harbor Laboratory Press, or Ausubel, F. M. et al. (1994) “Current Protocols in Molecular Biology”, John Wiley & Sons.)

Any plasmid or cosmid could be used. Of particular use were the plasmids pBR322 (Sutcliffe, J. G. (1979) Proc. Natl. Acad. Sci. USA, 75:3737-3741); pACYC177 (Change & Cohen (1978) J. Bacteriol 134:1141-1156), plasmids of the pBS series (pBSSK+, pBSSK− and others; Stratagene, LaJolla, USA), or cosmids as SuperCos1 (Stratagene, LaJolla, USA) or Lorist6 (Gibson, T. J., Rosenthal A. and Waterson, R. H. (1987) Gene 53:283-286. Gene libraries specifically for use in C. glutamicum may be constructed using plasmid pSL109 (Lee, H.-S, and A. J. Sinskey (1994) J. Microbiol. Biotechnol. 4: 256-263).

### Example 3

### DNA Sequencing and Computational Functional Analysis

Genomic libraries as described in Example 2 were used for DNA sequencing according to standard methods, in particular by the chain termination method using ABI377 sequencing machines (see e.g., Fleischman, R. D. et al. (1995) “Whole-genome Random Sequencing and Assembly of Haemophilus Influenzae Rd., Science, 269:496-512). Sequencing primers with the following nucleotide sequences were used: 5′-GGAAACAGTATGACCATG-3′ or 5′-GTAAAACGACGGCCAGT-3′.

### Example 4

### In Vivo Mutagenesis

In vivo mutagenesis of Corynebacterium glutamicum can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM: Washington.) Such strains are well known to one of ordinary skill in the art. The use of such strains is illustrated, for example, in Greener, A. and Callahan, M. (1994) Strategies 7: 32-34.

### Example 5

### DNA Transfer Between Escherichia coli and Corynebacterium Glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5:137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162:591-597, Martin J. F. et al. (1987) Biotechnology, 5:137-146 and Eikmanns, B. J. et al. (1991) Gene, 102:93-98).

Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebi, E. et al. (1989) FEMS Microbiol. Letters, 53:399-303) and in cases where special vectors are used, also by conjugation (as described e.g. in Schafer, A et al. (1990) J. Bacteriol. 172:1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166:1-19).

Genes may be overexpressed in C. glutamicum strains using plasmids which comprise pCG1 (U.S. Pat. No. 4,617,267) or fragments thereof, and optionally the gene for kanamycin resistance from TN903 (Grindley, N. D. and Joyce, C. M. (1980) Proc. Natl. Acad. Sci. USA 77(12): 7176-7180). In addition, genes may be overexpressed in C. glutamicum strains using plasmid pSL109 (Lee, H.-S, and A. J. Sinskey (1994) J. Microbiol Biotechnol. 4: 256-263).

Aside from the use of replicative plasmids, gene overexpression can also be achieved by integration into the genome. Genomic integration in C. glutamicum or other Corynebacterium or Brevibacterium species may be accomplished by well-known methods, such as homologous recombination with genomic region(s), restriction endonuclease mediated integration (REMI) (see, e.g., DE Patent 19823834), or through the use of transposons. It is also possible to modulate the activity of a gene of interest by modifying the regulatory regions (e.g., a promoter, a repressor, and/or an enhancer) by sequence modification, insertion, or deletion using site-directed methods (such as homologous recombination) or methods based on random events (such as transposon mutagenesis or REMI). Nucleic acid sequences which function as transcriptional terminators may also be inserted 3′ to the coding region of one or more genes of the invention; such terminators are well-known in the art and are described, for example, in Winnacker, E. L. (1987) From Genes to Clones—Introduction to Gene Technology. VCH: Weinheim.

### Example 6

### Assessment of the Expression of the Mutant Protein

Observations of the activity of a mutated protein in a transformed host cell rely on the fact that the mutant protein is expressed in a similar fashion and in a similar quantity to that of the wild-type protein. A useful method to ascertain the level of transcription of the mutant gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the mutant gene. Total cellular RNA can be prepared from Corynebacterium glutamicum by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or calorimetric label which may be readily detected. The presence and quantity of label observed indicates the presence and quantity of the desired mutant protein present in the cell.

### Example 7

### Growth of Genetically Modified Corynebacterium Glutamicum—Media and Culture Conditions

Genetically modified Corynebacteria are cultured in synthetic or natural growth media. A number of different growth media for Corynebacteria are both well-known and readily available (Lieb et al. (1989) Appl. Microbiol. Biotechnol., 32:205-210; von der Osten et al. (1998) Biotechnology Letters, 11:11-16; Patent DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of O0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/1 meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### Example 8

### In Vitro Analysis of the Function of Mutant Proteins

The determination of activities and kinetic parameters of enzymes is well established in the art. Experiments to determine the activity of any given altered enzyme must be tailored to the specific activity of the wild-type enzyme, which is well within the ability of one of ordinary skill in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: N.Y.; Walsh, (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymkinetik, 2^{nd} ed. VCH: Weinheim (ISBN 3527300325); Bergmeyer, H. U., Bergmeyer, J., Graβl, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### Example 9

### Analysis of Impact of Mutant Protein on the Production of the Desired Product

The effect of the genetic modification in C. glutamicum on production of a desired compound (such as an amino acid) can be assessed by growing the modified microorganism under suitable conditions (such as those described above) and analyzing the medium and/or the cellular component for increased production of the desired product (i.e., an amino acid). Such analysis techniques are well known to one of ordinary skill in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of fermentation, it is also possible to analyze other components of the metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

### Example 10

### Purification of the Desired Product from C. Glutamicum Culture

Recovery of the desired product from the C. glutamicum cells or supernatant of the above-described culture can be performed by various methods well known in the art. If the desired product is not secreted from the cells, the cells can be harvested from the culture by low-speed centrifugation, the cells can be lysed by standard techniques, such as mechanical force or sonication. The cellular debris is removed by centrifugation, and the supernatant fraction containing the soluble proteins is retained for further purification of the desired compound. If the product is secreted from the C. glutamicum cells, then the cells are removed from the culture by low-speed centrifugation, and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One of ordinary skill in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60:133-140; Malakhova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### Example 11

### Analysis of the Gene Sequences of the Invention

The comparison of sequences and determination of percent homology between two sequences are art-known techniques, and can be accomplished using a mathematical algorithm, such as the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to SES nucleic acid molecules of the invention. BLAST protein searches can be performed with the XBLAST program, score=50, wordlength=3 to obtain amino acid sequences homologous to SES protein molecules of the invention. To obtain gapped alignments for comparison purposes, Gapped BLAST can be utilized as described in Altschul et al, (1997) Nucleic Acids Res. 25(17):3389-3402. When utilizing BLAST and Gapped BLAST programs, one of ordinary skill in the art will know how to optimize the parameters of the program (e.g., XBLAST and NBLAST) for the specific sequence being analyzed.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM. described in Torelli and Robotti (1994) Comput. Appl. Biosci. 10:3-5; and FASTA, described in Pearson and Lipman (1988) P.N.A.S. 85:2444-8.

The percent homology between two amino acid sequences can also be accomplished using the GAP program in the GCG software package (available at http://www.gcg.com), using either a Blosum 62 matrix or a PAM250 matrix, and a gap weight of 12, 10, 8, 6, or 4 and a length weight of 2, 3, or 4. The percent homology between two nucleic acid sequences can be accomplished using the GAP program in the GCG software package, using standard parameters, such as a gap weight of 50 and a length weight of 3.

A comparative analysis of the gene sequences of the invention with those present in Genbank has been performed using techniques known in the art (see, e.g., Bexevanis and Ouellette, eds. (1998) Bioinformatics: A Practical Guide to the Analysis of Genes and Proteins. John Wiley and Sons: New York). The gene sequences of the invention were compared to genes present in Genbank in a three-step process. In a first step, a BLASTN analysis (e.g., a local alignment analysis) was performed for each of the sequences of the invention against the nucleotide sequences present in Genbank, and the top 500 hits were retained for further analysis. A subsequent FASTA search (e.g., a combined local and global alignment analysis, in which limited regions of the sequences are aligned) was performed on these 500 hits. Each gene sequence of the invention was subsequently globally aligned to each of the top three FASTA hits, using the GAP program in the GCG software package (using standard parameters). In order to obtain correct results, the length of the sequences extracted from Genbank were adjusted to the length of the query sequences by methods well-known in the art. The results of this analysis are set forth in Table 4. The resulting data is identical to that which would have been obtained had a GAP (global) analysis alone been performed on each of the genes of the invention in comparison with each of the references in Genbank, but required significantly reduced computational time as compared to such a database-wide GAP (global) analysis. Sequences of the invention for which no alignments above the cutoff values were obtained are indicated on Table 4 by the absence of alignment information. It will further be understood by one of ordinary skill in the art that the GAP alignment homology percentages set forth in Table 4 under the heading “% homology (GAP)” are listed in the European numerical format, wherein a ‘,’ represents a decimal point. For example, a value of “40,345” in this column represents “40.345%”.

### Example 12

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays, therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more C. glutamicum genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. These nucleic acid molecules can be labeled according to standard methods. In brief, nucleic acid molecules (e.g., mRNA molecules or DNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), supra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of C. glutamicum or other Corynebacteria. For example, studies of inter-strain variations based on individual transcript profiles and the identification of genes that are important for specific and/or desired strain properties such as pathogenicity, productivity and stress tolerance are facilitated by nucleic acid array methodologies. Also, comparisons of the profile of expression of genes of the invention during the course of a fermentation reaction are possible using nucleic acid array technology.

### Example 13

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of C. glutamicum (e.g., in comparison with the protein populations of other organisms), those proteins which are active under specific environmental or metabolic conditions (e.g., during fermentation, at high or low temperature, or at high or low pH), or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (WEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann et al. (1998) Electrophoresis 19: 3217-3221; Fountoulakis et al. (1998) Electrophoresis 19: 1193-1202; Langen et al. (1997) Electrophoresis 18: 1184-1192; Antelmann et al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵S-methionine, ³⁵S-cysteine, ¹⁴C-labelled amino acids, ¹⁵N-amino acids, ¹⁵NO₃ or ¹⁵NH₄⁺ or ¹³C-labelled amino acids) in the medium of C. glutamicum permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N- and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of C. glutamicum proteins by these techniques.

The information obtained by these methods can be used to compare patterns of protein presence, activity, or modification between different samples from various biological conditions (e.g., different organisms, time points of fermentation, media conditions, or different biotopes, among others). Data obtained from such experiments alone, or in combination with other techniques, can be used for various applications, such as to compare the behavior of various organisms in a given (e.g., metabolic) situation, to increase the productivity of strains which produce fine chemicals or to increase the efficiency of the production of fine chemicals.

### EQUIVALENTS

Those of ordinary skill in the art will recognize, or will be able to ascertain using no more than routine experimentation, many equivalents to the specific embodiments of the invention described herein. Such equivalents are intended to be encompassed by the following claims.

TABLE 1
GENES IN THE APPLICATION

| Nucleic | Amino | | | | | |
|---|---|---|---|---|---|---|
| Acid | Acid | | | | | |
| SEQ | SEQ | Identification | | NT | NT | |
| ID NO | ID NO | Code | Contig. | Start | Stop | Function |
| 1 | 2 | RXA01278 | GR00369 | 2425 | 299 | Protein Translation Elongation Factor G (EF-G) |
| 3 | 4 | RXA01913 | GR00547 | 1856 | 2680 | Protein translation Elongation Factor Ts (EF-Ts) |
| 5 | 6 | RXN01559 | VV0171 | 7795 | 5954 | PROTEIN-EXPORT MEMBRANE PROTEIN SECD |
| 7 | 8 | F RXA00935 | GR00254 | 654 | 4 | PROTEIN-EXPORT MEMBRANE PROTEIN SECD |
| 9 | 10 | F RXA01559 | GR00434 | 1983 | 1741 | PROTEIN-EXPORT MEMBRANE PROTEIN SECD |
| 11 | 12 | RXA01558 | GR00434 | 1735 | 527 | PROTEIN-EXPORT MEMBRANE PROTEIN SECF |
| 13 | 14 | RXA02429 | GR00707 | 4823 | 7111 | PREPROTEIN TRANSLOCASE SECA SUBUNIT |
| 15 | 16 | RXA02748 | GR00764 | 2434 | 4074 | SIGNAL RECOGNITION PARTICLE PROTEIN |
| 17 | 18 | RXA01355 | GR00393 | 2877 | 3662 | SIGNAL PEPTIDASE I (EC 3.4.21.89) |
| 19 | 20 | RXA00107 | GR00014 | 17940 | 18176 | GLUTAREDOXIN-LIKE PROTEIN NRDH |
| 21 | 22 | RXA01613 | GR00449 | 7055 | 5841 | GLUTATHIONE REDUCTASE (EC 1.6.4.2) |
| 23 | 24 | RXA00539 | GR00139 | 1460 | 1936 | GLUTATHIONE PEROXIDASE (EC 1.11.1.9) |

Genes and enzymes involved in DNA uptake, repair and recombination

| | | | | | | |
|---|---|---|---|---|---|---|
| 25 | 26 | RXA01020 | GR00291 | 998 | 1744 | URACIL-DNA GLYCOSYLASE (EC 3.2.2.—) |
| 27 | 28 | RXN00484 | VV0086 | 47365 | 46286 | DEOXYRIBODIPYRIMIDINE PHOTOLYASE (EC 4.1.99.3) |
| 29 | 30 | F RXA00484 | GR00119 | 21602 | 20568 | DEOXYRIBODIPYRIMIDINE PHOTOLYASE (EC 4.1.99.3) |
| 31 | 32 | RXA02476 | GR00715 | 10514 | 9636 | A/G-SPECIFIC ADENINE GLYCOSYLASE (EC 3.2.2.—) |
| 33 | 34 | RXA00102 | GR00014 | 11288 | 10521 | FORMAMIDOPYRIMIDINE-DNA GLYCOSYLASE (EC 3.2.2.23) |
| 35 | 36 | RXN01670 | VV0079 | 18911 | 18105 | FORMAMIDOPYRIMIDINE-DNA GLYCOSYLASE (EC 3.2.2.23) |
| 37 | 38 | F RXA01670 | GR00466 | 3 | 614 | FORMAMIDOPYRIMIDINE-DNA GLYCOSYLASE (EC 3.2.2.23) |
| 39 | 40 | RXA02078 | GR00628 | 8170 | 9027 | FORMAMIDOPYRIMIDINE-DNA GLYCOSYLASE (EC 3.2.2.23) |
| 41 | 42 | RXA01596 | GR00447 | 4370 | 6148 | DNA REPAIR PROTEIN RECN |
| 43 | 44 | RXA01493 | GR00423 | 7530 | 6220 | DNA-DAMAGE-INDUCIBLE PROTEIN F |
| 45 | 46 | RXA02671 | GR00753 | 11718 | 12296 | DNA REPAIR PROTEIN RADA HOMOLOG |
| 47 | 48 | RXN02291 | VV0127 | 18678 | 18025 | ALKB PROTEIN (DNA repair - alkylated DNA) |
| 49 | 50 | F RXA02291 | GR00662 | 1518 | 865 | DNA repair gene specific for alkylated DNA |
| 51 | 52 | RXN01733 | VV0221 | 70 | 1251 | RECF PROTEIN |
| 53 | 54 | F RXA01733 | GR00492 | 2 | 544 | RECF PROTEIN |
| 55 | 56 | RXA01252 | GR00365 | 643 | 1296 | RECOMBINATION PROTEIN RECR |
| 57 | 58 | RXA01878 | GR00537 | 1239 | 2117 | DIMETHYLADENOSINE TRANSFERASE (EC 2.1.1.—) |
| 59 | 60 | RXA01556 | GR00433 | 1 | 849 | METHYLPHOSPHOTRIESTER-DNA ALKYLTRANSFERASE |
| 61 | 62 | RXA00053 | GR00008 | 8162 | 8554 | MUTATOR MUTT PROTEIN (7,8-DIHYDRO-8-OXOGUANINE- |
| | | | | | | TRIPHOSPHATASE) (8-OXO-DGTPASE) (EC 3.6.1.—) |
| 63 | 64 | RXA00280 | GR00043 | 4196 | 4696 | MUTATOR MUTT PROTEIN (7,8-DIHYDRO-8-OXOGUANINE- |
| | | | | | | TRIPHOSPHATASE) (8-OXO-DGTPASE) (EC 3.6.1.—) |
| 65 | 66 | RXA00333 | GR00057 | 16166 | 16699 | MUTATOR MUTT PROTEIN (7,8-DIHYDRO-8-OXOGUANINE- |
| | | | | | | TRIPHOSPHATASE) (8-OXO-DGTPASE) (EC 3.6.1.—) |
| 67 | 68 | RXA02110 | GR00632 | 3641 | 4258 | MUTATOR MUTT PROTEIN (7,8-DIHYDRO-8-OXOGUANINE- |
| | | | | | | TRIPHOSPHATASE) (8-OXO-DGTPASE) (EC 3.6.1.—) |
| 69 | 70 | RXA02290 | GR00662 | 693 | 295 | DNA-3-METHYLADENINE GLYCOSIDASE I (EC 3.2.2.20) |
| 71 | 72 | RXA02557 | GR00731 | 3766 | 3179 | DNA-3-METHYLADENINE GLYCOSIDASE I (EC 3.2.2.20) |
| 73 | 74 | RXA02130 | GR00638 | 1 | 87 | DNA REPAIR HELICASE RAD25 |
| 75 | 76 | RXA02742 | GR00763 | 12384 | 10036 | Hypothetical DNA Repair Helicase |
| 77 | 78 | RXA02445 | GR00709 | 9362 | 11050 | ATP-DEPENDENT DNA HELICASE RECG |
| 79 | 80 | RXA00927 | GR00253 | 1606 | 518 | HOLLIDAY JUNCTION DNA HELICASE RUVB |
| 81 | 82 | RXA00928 | GR00253 | 2233 | 1616 | HOLLIDAY JUNCTION DNA HELICASE RUVA |
| 83 | 84 | RXN00172 | VV0187 | 7949 | 8560 | RESOLVASE |
| 85 | 86 | F RXA00172 | GR00027 | 455 | 6 | RESOLVASE |
| 87 | 88 | RXA00184 | GR00028 | 8239 | 9411 | DNA repair exonuclease |
| 89 | 90 | RXA00019 | GR00002 | 14399 | 16258 | SINGLE-STRANDED-DNA-SPECIFIC EXONUCLEASE RECJ (EC 3.1.—.—) |
| 91 | 92 | RXA00929 | GR00253 | 2938 | 2276 | CROSSOVER JUNCTION ENDODEOXYRIBONUCLEASE RUVC (EC 3.1.22.4) |
| 93 | 94 | RXA02251 | GR00654 | 18367 | 18666 | EXCINUCLEASE ABC SUBUNIT C |
| 95 | 96 | RXA02252 | GR00654 | 18632 | 20455 | EXCINUCLEASE ABC SUBUNIT C |
| 97 | 98 | RXN02416 | VV0116 | 10457 | 7629 | EXCINUCLEASE ABC SUBUNIT A |
| 99 | 100 | F RXA02416 | GR00705 | 3 | 2642 | EXCINUCLEASE ABC SUBUNIT A |
| 101 | 102 | RXA02563 | GR00732 | 1515 | 2246 | Excinuclease ATPase subunit |
| 103 | 104 | RXA02731 | GR00762 | 3263 | 5359 | EXCINUCLEASE ABC SUBUNIT B |
| 105 | 106 | RXA00998 | GR00283 | 2871 | 2410 | COMA OPERON PROTEIN 2 |
| 107 | 108 | RXN02386 | VV0176 | 368 | 826 | COME OPERON PROTEIN 1 |
| 109 | 110 | F RXA02386 | GR00693 | 1180 | 776 | COME OPERON PROTEIN 1, DNA binding and uptake (competence) |
| 111 | 112 | RXN02388 | VV0176 | 826 | 2487 | COME OPERON PROTEIN 3 |
| 113 | 114 | F RXA02385 | GR00693 | 776 | 6 | COME OPERON PROTEIN 3, DNA binding and uptake (competence) |
| 115 | 116 | F RXA02388 | GR00694 | 1770 | 925 | COME OPERON PROTEIN 3, DNA binding and uptake (competence) |
| 117 | 118 | RXA01975 | GR00571 | 242 | 2137 | PUTATIVE TYPE II RESTRICTION ENDONUCLEASE AND PUTATIVE |
| | | | | | | TYPE I OR TYPE III RESTRICTION ENDONUCLEASE GENES, |
| | | | | | | COMPLETE CDS |
| 119 | 120 | RXA01954 | GR00562 | 3326 | 4165 | TYPE III RESTRICTION-MODIFICATION SYSTEM |
| | | | | | | ECOP15I ENZYME MOD (EC 2.1.1.72) |
| 121 | 122 | RXA02236 | GR00654 | 4249 | 4566 | integration host factor |
| 123 | 124 | RXN01795 | VV0093 | 722 | 1318 | MODIFICATION METHYLASE (EC 2.1.1.73) |
| 125 | 126 | RXN02267 | VV0020 | 10928 | 10056 | DNA (CYTOSINE-5)-METHYLTRANSFERASE (EC 2.1.1.37) |
| 127 | 128 | RXA02988 | VV0093 | 231 | 836 | MODIFICATION METHYLASE SCRFI-A (EC 2.1.1.73) |
| 129 | 130 | RXN00127 | VV0124 | 9789 | 10253 | COMPETENCE PROTEIN F |
| 131 | 132 | RXN02938 | VV0054 | 23357 | 24097 | MUTATOR MUTT PROTEIN (7,8-DIHYDRO-8-OXOGUANINE- |
| | | | | | | TRIPHOSPHATASE) (8-OXO-DGTPASE) |
| 133 | 134 | RXN03102 | VV0067 | 5253 | 4762 | PUTATIVE COMPETENCE-DAMAGE PROTEIN |
| 135 | 136 | RXN03118 | VV0093 | 1330 | 2139 | PUTATIVE TYPE II RESTRICTION ENDONUCLEASE AND PUTATIVE |
| | | | | | | TYPE I OR TYPE III RESTRICTION ENDONUCLEASE GENES, |
| | | | | | | COMPLETE CDS |
| 137 | 138 | RXN02989 | VV0073 | 118 | 1257 | RECA PROTEIN |
| 139 | 140 | RXN03168 | VV0327 | 1777 | 695 | RIBONUCLEASE BN (EC 3.1.—.—) |
| 141 | 142 | RXN02431 | VV0090 | 1 | 876 | UMUC PROTEIN |
| 143 | 144 | RXN02985 | VV0009 | 1182 | 850 | EBSC PROTEIN |
| 145 | 146 | RXN02986 | VV0009 | 801 | 664 | EBSC PROTEIN |
| 147 | 148 | RXS00061 | VV0044 | 4256 | 1590 | DNA POLYMERASE I (EC 2.7.7.7) |
| 149 | 150 | RXS00212 | VV0096 | 12413 | 10854 | DNA LIGASE (EC 6.5.1.2) |
| 151 | 152 | RXS00213 | VV0096 | 12894 | 12322 | DNA LIGASE (EC 6.5.1.2) |
| 153 | 154 | RXS00724 | VV0052 | 1217 | 3193 | ATP-DEPENDENT DNA HELICASE RECG (EC 3.6.1.—) |
| 155 | 156 | RXS00823 | VV0054 | 22014 | 22793 | ENDONUCLEASE III (EC 4.2.99.18) |
| 157 | 158 | RXS00898 | VV0140 | 4755 | 5543 | EXODEOXYRIBONUCLEASE III (EC 3.1.11.2) |
| 159 | 160 | RXS01066 | VV0099 | 21112 | 21837 | DNA REPAIR PROTEIN RECO |
| 161 | 162 | RXS02145 | VV0300 | 12248 | 13864 | ENDONUCLEASE III (EC 4.2.99.18) |
| 163 | 164 | RXS02476 | VV0008 | 49453 | 48575 | A/G-SPECIFIC ADENINE GLYCOSYLASE (EC 3.2.2.—) |
| 165 | 166 | RXS02990 | VV0073 | 1352 | 1948 | REGULATORY PROTEIN RECX |
| 167 | 168 | RXS03098 | VV0064 | 2100 | 2723 | DNA alkylation repair enzyme |
| 169 | 170 | RXS03175 | VV0331 | 1248 | 466 | EXODEOXYRIBONUCLEASE III (EC 3.1.11.2) |

Transposon, IS elements, Transposase, Integrase

| | | | | | | |
|---|---|---|---|---|---|---|
| 171 | 172 | RXN03069 | VV0039 | 5816 | 4734 | INTEGRASE |
| 173 | 174 | F RXA02890 | GR10027 | 112 | 1194 | INTEGRASE |
| 175 | 176 | RXA01601 | GR00447 | 11128 | 12039 | INTEGRASE/RECOMBINASE XERD |
| 177 | 178 | RXA01228 | GR00355 | 1668 | 1883 | TRANSPOSONS TN1721 AND TN4653 RESOLVASE |
| 179 | 180 | RXN03130 | VV0123 | 14262 | 15569 | DNA, TRANSPOSABLE ELEMENT IS31831 |
| 181 | 182 | RXN01969 | VV0155 | 139 | 504 | DNA, TRANSPOSABLE ELEMENT IS31831 |
| 183 | 184 | F RXA00263 | GR00040 | 2243 | 936 | DNA, TRANSPOSABLE ELEMENT IS31831 |
| 185 | 186 | RXN01541 | VV0015 | 56012 | 56788 | PLASMID PASU1 TRANSPOSASE |
| 187 | 188 | F RXA01541 | GR00428 | 3865 | 3095 | PLASMID PASU1 TRANSPOSASE |
| 189 | 190 | RXA02590 | GR00741 | 14837 | 13902 | INSERTION ELEMENT IS1415 TRANSPOSASE (ISTA) AND |
| | | | | | | HELPER PROTEIN(ISTB) GENES, COMPLETE CDS |
| 191 | 192 | RXA00016 | GR00002 | 8857 | 7964 | IS3 RELATED INSERTION ELEMENT |
| 193 | 194 | RXA00265 | GR00040 | 2840 | 3289 | TRANSPOSASE |
| 195 | 196 | RXA00938 | GR00256 | 670 | 927 | TRANSPOSASE |
| 197 | 198 | RXA01264 | GR00367 | 12003 | 11788 | TRANSPOSASE |
| 199 | 200 | RXA01265 | GR00367 | 12616 | 12467 | TRANSPOSASE |
| 201 | 202 | RXA01327 | GR00386 | 753 | 896 | TRANSPOSASE |
| 203 | 204 | RXA01328 | GR00386 | 991 | 1365 | TRANSPOSASE |
| 205 | 206 | RXA01329 | GR00386 | 1407 | 1697 | TRANSPOSASE |
| 207 | 208 | RXA01443 | GR00418 | 13570 | 12740 | TRANSPOSASE |
| 209 | 210 | RXA01444 | GR00418 | 13928 | 13662 | TRANSPOSASE |
| 211 | 212 | RXA01648 | GR00457 | 829 | 461 | TRANSPOSASE |
| 213 | 214 | RXA01649 | GR00457 | 1260 | 841 | TRANSPOSASE |
| 215 | 216 | RXA01650 | GR00457 | 1437 | 1324 | TRANSPOSASE |
| 217 | 218 | RXA01651 | GR00457 | 1618 | 1484 | TRANSPOSASE |
| 219 | 220 | RXN01680 | VV0179 | 17470 | 17060 | TRANSPOSASE |
| 221 | 222 | F RXA01680 | GR00467 | 9590 | 9180 | TRANSPOSASE |
| 223 | 224 | RXN01784 | VV0084 | 13161 | 12580 | TRANSPOSASE |
| 225 | 226 | F RXA01784 | GR00505 | 3 | 551 | TRANSPOSASE |
| 227 | 228 | RXA01862 | GR00529 | 4961 | 6166 | TRANSPOSASE |
| 229 | 230 | RXA01953 | GR00562 | 928 | 548 | TRANSPOSASE |
| 231 | 232 | RXA01998 | GR00589 | 1345 | 2052 | TRANSPOSASE |
| 233 | 234 | RXA02837 | GR00829 | 179 | 6 | TRANSPOSASE |
| 235 | 236 | RXA00005 | GR00001 | 4724 | 6331 | TRANSPOSASE |
| 237 | 238 | RXA00017 | GR00002 | 9150 | 8857 | TRANSPOSASE |
| 239 | 240 | RXA00057 | GR00009 | 2491 | 2393 | TRANSPOSASE |
| 241 | 242 | RXA00227 | GR00032 | 27991 | 27194 | TRANSPOSASE |
| 243 | 244 | RXA01819 | GR00515 | 8287 | 7841 | transposase |
| 245 | 246 | RXN03052 | VV0024 | 5310 | 4555 | INTEGRASE |
| 247 | 248 | RXN02915 | VV0135 | 43798 | 44175 | TRANSPOSASE |
| 249 | 250 | RXN02919 | VV0084 | 14953 | 15486 | TRANSPOSASE |
| 251 | 252 | RXN03033 | VV0012 | 3942 | 5099 | TRANSPOSASE |
| 253 | 254 | RXN03035 | VV0013 | 667 | 1824 | TRANSPOSASE |
| 255 | 256 | RXN03049 | VV0020 | 29926 | 28985 | TRANSPOSASE |
| 257 | 258 | RXN03070 | VV0039 | 8897 | 8070 | TRANSPOSASE |
| 259 | 260 | RXN03121 | VV0101 | 645 | 4 | TRANSPOSASE |
| 261 | 262 | RXN03161 | VV0193 | 884 | 1267 | TRANSPOSASE |
| 263 | 264 | RXN03165 | VV0312 | 1562 | 1242 | TRANSPOSASE |
| 265 | 266 | RXN00083 | VV0048 | 3416 | 3117 | TRANSPOSASE |
| 267 | 268 | RXN02004 | VV0290 | 588 | 382 | TRANSPOSASE |
| 269 | 270 | RXN02287 | VV0127 | 69201 | 69752 | TRANSPOSON TN2501 RESOLVASE |
| 271 | 272 | RXN02963 | VV0102 | 6547 | 5240 | DNA, TRANSPOSABLE ELEMENT IS31831 |

Aminoacyl-tRNA synthetases/tRNAs and tRNA metabolism

| | | | | | | |
|---|---|---|---|---|---|---|
| 273 | 274 | RXA02788 | GR00777 | 2359 | 5022 | ALANYL-TRNA SYNTHETASE (EC 6.1.1.7) |
| 275 | 276 | RXN00975 | VV0149 | 7820 | 9469 | ARGINYL-TRNA SYNTHETASE (EC 6.1.1.19) |
| 277 | 278 | F RXA00975 | GR00275 | 780 | 4 | POSSIBLE ARGINYL-TRNA SYNTHETASE (EC 6.1.1.19) |
| 279 | 280 | F RXA00976 | GR00275 | 1423 | 824 | POSSIBLE ARGINYL-TRNA SYNTHETASE (EC 6.1.1.19) |
| 281 | 282 | RXN01730 | VV0137 | 1709 | 6 | ASPARTYL-TRNA SYNTHETASE (EC 6.1.1.12) |
| 283 | 284 | F RXA01730 | GR00490 | 298 | 1974 | ASPARTYL-TRNA SYNTHETASE (EC 6.1.1.12) |
| 285 | 286 | RXA00314 | GR00053 | 5406 | 4027 | CYSTEINYL-TRNA SYNTHETASE (EC 6.1.1.16) |
| 287 | 288 | RXA02204 | GR00646 | 8756 | 7497 | CYSTEINYL-TRNA SYNTHETASE (EC 6.1.1.16) |
| 289 | 290 | RXA01124 | GR00312 | 2 | 1510 | GLUTAMYL-TRNA SYNTHETASE (EC 6.1.1.17) |
| 291 | 292 | RXN00458 | VV0076 | 8169 | 8804 | GLUTAMYL-TRNA SYNTHETASE (EC 6.1.1.17) |
| 293 | 294 | F RXA00458 | GR00115 | 232 | 5 | GLUTAMYL-TRNA SYNTHETASE (EC 6.1.1.17) |
| 295 | 296 | RXA00069 | GR00011 | 2782 | 1400 | GLYCYL-TRNA SYNTHETASE (EC 6.1.1.14) |
| 297 | 298 | RXA01852 | GR00525 | 4873 | 3587 | HISTIDYL-TRNA SYNTHETASE (EC 6.1.1.21) |
| 299 | 300 | RXA02726 | GR00760 | 4530 | 1597 | ISOLEUCYL-TRNA SYNTHETASE (EC 6.1.1.5) |
| 301 | 302 | RXN00966 | VV0262 | 543 | 4 | LEUCYL-TRNA SYNTHETASE (EC 6.1.1.4) |
| 303 | 304 | F RXA00966 | GR00271 | 533 | 6 | LEUCYL-TRNA SYNTHETASE (EC 6.1.1.4) |
| 305 | 306 | RXN01061 | VV0079 | 1 | 1038 | LEUCYL-TRNA SYNTHETASE (EC 6.1.1.4) |
| 307 | 308 | F RXA01864 | GR00531 | 474 | 4 | LEUCYL-TRNA SYNTHETASE (EC 6.1.1.4) |
| 309 | 310 | F RXA01061 | GR00296 | 10974 | 10567 | LEUCYL-TRNA SYNTHETASE (EC 6.1.1.4) |
| 311 | 312 | RXA00968 | GR00272 | 1007 | 6 | LEUCYL-TRNA SYNTHETASE (EC 6.1.1.4) |
| 313 | 314 | RXA01522 | GR00424 | 26014 | 27591 | LYSYL-TRNA SYNTHETASE (EC 6.1.1.6) |
| 315 | 316 | RXA02015 | GR00609 | 152 | 670 | METHIONYL-TRNA SYNTHETASE (EC 6.1.1.10) |
| 317 | 318 | RXA01582 | GR00440 | 1619 | 2707 | PHENYLALANYL-TRNA SYNTHETASE ALPHA CHAIN (EC 6.1.1.20) |
| 319 | 320 | RXN01583 | VV0122 | 19884 | 17542 | PHENYLALANYL-TRNA SYNTHETASE BETA CHAIN (EC 6.1.1.20) |
| 321 | 322 | F RXA01583 | GR00440 | 2914 | 4629 | PHENYLALANYL-TRNA SYNTHETASE BETA CHAIN (EC 6.1.1.20) |
| 323 | 324 | F RXA01717 | GR00487 | 1000 | 719 | PHENYLALANYL-TRNA SYNTHETASE BETA CHAIN (EC 6.1.1.20) |
| 325 | 326 | RXN01938 | VV0139 | 19106 | 20533 | PROLYL-TRNA SYNTHETASE (EC 6.1.1.15) |
| 327 | 328 | F RXA01938 | GR00556 | 94 | 1008 | PROLYL-TRNA SYNTHETASE (EC 6.1.1.15) |
| 329 | 330 | RXA02692 | GR00754 | 15485 | 16750 | SERYL-TRNA SYNTHETASE (EC 6.1.1.11) |
| 331 | 332 | RXA02167 | GR00640 | 13255 | 14514 | TYROSYL-TRNA SYNTHETASE 1 (EC 6.1.1.1) |
| 333 | 334 | RXA02509 | GR00721 | 2 | 1972 | THREONYL-TRNA SYNTHETASE (EC 6.1.1.3) |
| 335 | 336 | RXN03169 | VV0327 | 2326 | 1889 | TRYPTOPHANYL-TRNA SYNTHETASE (EC 6.1.1.2) |
| 337 | 338 | F RXA02860 | GR10006 | 2 | 439 | TRYPTOPHANYL-TRNA SYNTHETASE (EC 6.1.1.2) |
| 339 | 340 | RXN03078 | VV0045 | 484 | 5 | TRYPTOPHANYL-TRNA SYNTHETASE (EC 6.1.1.2) |
| 341 | 342 | F RXA02866 | GR10007 | 3992 | 4471 | TRYPTOPHANYL-TRNA SYNTHETASE (EC 6.1.1.2) |
| 343 | 344 | RXN00985 | VV0123 | 6747 | 9455 | VALYL-TRNA SYNTHETASE (EC 6.1.1.9) |
| 345 | 346 | F RXA00985 | GR00279 | 498 | 4 | VALYL-TRNA SYNTHETASE (EC 6.1.1.9) |
| 347 | 348 | F RXA01347 | GR00391 | 3036 | 5084 | VALYL-TRNA SYNTHETASE (EC 6.1.1.9) |
| 349 | 350 | RXN00454 | VV0076 | 3497 | 4789 | QUEUINE TRNA-RIBOSYLTRANSFERASE (EC 2.4.2.29) |
| 351 | 352 | F RXA00454 | GR00112 | 869 | 6 | QUEUINE TRNA-RIBOSYLTRANSFERASE (EC 2.4.2.29) |
| 353 | 354 | RXN01490 | VV0139 | 38695 | 37805 | TRNA PSEUDOURIDINE SYNTHASE B (EC 4.2.1.70) |
| 355 | 356 | F RXA01490 | GR00423 | 3442 | 4332 | TRNA PSEUDOURIDINE 55 SYNTHASE |
| 357 | 358 | RXA01621 | GR00452 | 473 | 1912 | TRNA NUCLEOTIDYLTRANSFERASE (EC 2.7.7.25) |
| 359 | 360 | RXN01704 | VV0191 | 1 | 1077 | TRNA (URACIL-5-)-METHYLTRANSFERASE (EC 2.1.1.35) |
| 361 | 362 | F RXA01704 | GR00480 | 3 | 818 | TRNA (URACIL-5-)-METHYLTRANSFERASE (EC 2.1.1.35) |
| 363 | 364 | RXA02523 | GR00725 | 1587 | 2405 | TRNA (GUANINE-N1)-METHYLTRANSFERASE (EC 2.1.1.31) |
| 365 | 366 | RXA02243 | GR00654 | 11114 | 12058 | METHIONYL-TRNA FORMYLTRANSFERASE (EC 2.1.2.9) |
| 367 | 368 | RXA00217 | GR00032 | 17389 | 16295 | PROBABLE TRNA (5-METHYLAMINOMETHYL-2-THIOURIDYLATE)- |
| | | | | | | METHYLTRANSFERASE (EC 2.1.1.61) |
| 369 | 370 | RXA01223 | GR00354 | 4156 | 3545 | PEPTIDYL-TRNA HYDROLASE (EC 3.1.1.29) |
| 371 | 372 | RXA01226 | GR00354 | 7416 | 6973 | PEPTIDYL-TRNA HYDROLASE (EC 3.1.1.29) |
| 373 | 374 | RXA00209 | GR00032 | 9592 | 8102 | L-glutamyl-tRNA(‘Gln)-dependent amidotransferase subunit A (EC 6.3.5.—) |
| 375 | 376 | RXA00210 | GR00032 | 9897 | 9601 | L-glutamyl-tRNA(‘Gln)-dependent amidotransferase subunit C (EC 6.3.5.—) |
| 377 | 378 | RXA02686 | GR00754 | 11266 | 10130 | L-glutamyl-tRNA(‘Gln)-dependent amidotransferase subunit A (EC 6.3.5.—) |
| 379 | 380 | RXA02625 | GR00747 | 791 | 6 | L-glutamyl-tRNA(‘Gln)-dependent amidotransferase subunit B (EC 6.3.5.—) |
| 381 | 382 | RXA01398 | GR00408 | 7645 | 7010 | L-glutamyl-tRNA(‘Gln)-dependent amidotransferase subunit B (EC 6.3.5.—) |
| 383 | 384 | RXA02228 | GR00653 | 1876 | 2778 | TRNA DELTA(2)-ISOPENTENYLPYROPHOSPHATE |
| | | | | | | TRANSFERASE (EC 2.5.1.8) |
| 385 | 386 | RXA02502 | GR00720 | 15510 | 16901 | GLUTAMYL-TRNA REDUCTASE (EC 1.2.1.—) |
| 387 | 388 | RXA02182 | GR00641 | 17875 | 18648 | GLUTAMINE CYCLOTRANSFERASE PRECURSOR (EC 2.3.2.5), |
| | | | | | | Glutaminyl-tRNA cyclotransferase |
| 389 | 390 | RXN00211 | VV0096 | 10126 | 10788 | L-glutamyl-tRNA('Gln)-dependent amidotransferase subunit B (EC 6.3.5.—) |
| 391 | 392 | RXN00669 | VV0005 | 38825 | 39706 | PSEUDOURIDYLATE SYNTHASE I (EC 4.2.1.70) |
| 393 | 394 | RXN02651 | VV0090 | 6842 | 7771 | SFHB PROTEIN |

Transcription

| | | | | | | |
|---|---|---|---|---|---|---|
| 395 | 396 | RXA01344 | GR00390 | 2551 | 5 | DNA-DIRECTED RNA POLYMERASE BETA CHAIN (EC 2.7.7.6) |
| 397 | 398 | RXA01387 | GR00407 | 372 | 4 | DNA-DIRECTED RNA POLYMERASE BETA′ CHAIN (EC 2.7.7.6) |
| 399 | 400 | RXA01388 | GR00407 | 590 | 459 | DNA-DIRECTED RNA POLYMERASE BETA CHAIN (EC 2.7.7.6) |
| 401 | 402 | RXA01283 | GR00369 | 7109 | 5817 | DNA-DIRECTED RNA POLYMERASE BETA′ CHAIN (EC 2.7.7.6) |
| 403 | 404 | RXA01433 | GR00417 | 9606 | 9004 | SIGMA FACTOR |
| 405 | 406 | RXA02456 | GR00712 | 1127 | 510 | RNA POLYMERASE SIGMA-H FACTOR |
| 407 | 408 | RXA00304 | GR00051 | 696 | 4 | RNA POLYMERASE SIGMA FACTOR |
| 409 | 410 | RXA00495 | GR00123 | 1210 | 1773 | PUTATIVE RNA POLYMERASE SIGMA FACTOR CY78.15 |
| 411 | 412 | RXA00532 | GR00137 | 3 | 587 | PROBABLE RNA POLYMERASE SIGMA FACTOR CY49.08 |
| 413 | 414 | RXA01530 | GR00426 | 1724 | 1083 | RNA POLYMERASE SIGMA FACTOR RPOD |
| 415 | 416 | RXA01531 | GR00426 | 2565 | 1549 | RNA POLYMERASE SIGMA FACTOR RPOD |
| 417 | 418 | RXA02065 | GR00626 | 5348 | 5995 | EXTRACYTOPLASMIC FUNCTION ALTERNATIVE SIGMA FACTOR |
| 419 | 420 | RXA00588 | GR00156 | 13672 | 14193 | TRANSCRIPTION ELONGATION FACTOR GREA |
| 421 | 422 | RXN01724 | VV0037 | 2128 | 809 | TRANSCRIPTION TERMINATION FACTOR RHO |
| 423 | 424 | F RXA01723 | GR00488 | 6600 | 7436 | TRANSCRIPTION TERMINATION FACTOR RHO |
| 425 | 426 | F RXA01724 | GR00488 | 7429 | 7812 | TRANSCRIPTION TERMINATION FACTOR RHO |
| 427 | 428 | RXN01725 | VV0037 | 825 | 619 | TRANSCRIPTION TERMINATION FACTOR RHO |
| 429 | 430 | F RXA01725 | GR00488 | 7897 | 8004 | TRANSCRIPTION TERMINATION FACTOR RHO |
| 431 | 432 | RXA01726 | GR00488 | 8000 | 8572 | TRANSCRIPTION TERMINATION FACTOR RHO |
| 433 | 434 | RXA00736 | GR00199 | 1 | 1887 | TRANSCRIPTION-REPAIR COUPLING FACTOR |
| 435 | 436 | RXN00737 | VV0094 | 2673 | 1681 | TRANSCRIPTION-REPAIR COUPLING FACTOR |
| 437 | 438 | F RXA00737 | GR00200 | 1 | 480 | TRANSCRIPTION-REPAIR COUPLING FACTOR |
| 439 | 440 | RXN01872 | VV0248 | 2141 | 2968 | TRANSCRIPTIONAL REGULATORY PROTEIN |
| 441 | 442 | F RXA01872 | GR00535 | 768 | 4 | TRANSCRIPTIONAL REGULATORY PROTEIN |
| 443 | 444 | RXA02413 | GR00703 | 3029 | 2538 | PAPX PROTEIN, transcriptional regulator |
| 445 | 446 | RXN01404 | VV0278 | 3 | 1001 | TRANSCRIPTION REGULATORY PROTEIN PEPR1 |
| 447 | 448 | RXN02827 | VV0350 | 428 | 6 | TRANSCRIPTION-REPAIR COUPLING FACTOR |
| 449 | 450 | RXN02732 | VV0145 | 3915 | 3475 | Putative transcription factors |
| 451 | 452 | RXN01671 | VV0079 | 17865 | 16717 | RTCB PROTEIN |
| 453 | 454 | RXS00671 | VV0005 | 37121 | 38134 | DNA-DIRECTED RNA POLYMERASE ALPHA CHAIN (EC 2.7.7.6) |
| 455 | 456 | RXS02760 | VV0025 | 31807 | 32760 | TRANSCRIPTION ANTITERMINATION PROTEIN NUSG |
| 457 | 458 | RXS02830 | VV0168 | 3 | 650 | Helix-turn-helix domain-containing transcription regulator |
| 459 | 460 | RXS03207 | | | | RNA POLYMERASE SIGMA FACTOR |

Translation

| | | | | | | |
|---|---|---|---|---|---|---|
| 461 | 462 | RXA02418 | GR00705 | 5101 | 5667 | Bacterial Protein Translation Initiation Factor 3 (IF-3) |
| 463 | 464 | RXN01496 | VV0139 | 29945 | 32956 | Protein Translation Initiation Factor 2 (IF-2) |
| 465 | 466 | F RXA00755 | GR00203 | 1280 | 6 | Protein Translation Initiation Factor 2 (IF-2) |
| 467 | 468 | F RXA01496 | GR00423 | 10908 | 9181 | Protein Translation Initiation Factor 2 (IF-2) |
| 469 | 470 | RXA00677 | GR00178 | 1624 | 1839 | Bacterial Protein Translation Initiation Factor 1 (IF-1) |
| 471 | 472 | RXN01284 | VV0212 | 570 | 4 | Bacterial Protein Translation Elongation Factor Tu (EF-TU) |
| 473 | 474 | F RXA01284 | GR00370 | 510 | 4 | Bacterial Protein Translation Elongation FactorTu (EF-TU) |
| 475 | 476 | RXA00138 | GR00022 | 1914 | 2474 | Protein Translation Elongation Factor P (EF-P) |
| 477 | 478 | RXA00331 | GR00057 | 15141 | 14785 | Hypothetical Translational Inhibitor Protein |
| 479 | 480 | RXA02822 | GR00803 | 1 | 570 | Bacterial Peptide Chain Release Factor 1 (RF-1) |
| 481 | 482 | RXA00011 | GR00002 | 2739 | 2383 | Bacterial Peptide Chain Release Factor 2 (RF-2) |
| 483 | 484 | RXA00012 | GR00002 | 3487 | 2612 | Bacterial Peptide Chain Release Factor 2 (RF-2) |
| 485 | 486 | RXN01926 | VV0284 | 1 | 741 | PEPTIDE CHAIN RELEASE FACTOR 3 |
| 487 | 488 | F RXA01926 | GR00554 | 1 | 672 | PEPTIDE CHAIN RELEASE FACTOR 3 |
| 489 | 490 | RXN02002 | VV0111 | 141 | 518 | PEPTIDE CHAIN RELEASE FACTOR 3 |
| 491 | 492 | F RXA02002 | GR00592 | 383 | 6 | PEPTIDE CHAIN RELEASE FACTOR 3 |
| 493 | 494 | RXA00896 | GR00244 | 2884 | 3522 | POLYPEPTIDE DEFORMYLASE (EC 3.5.1.31) |
| 495 | 496 | RXA02242 | GR00654 | 10585 | 11091 | POLYPEPTIDE DEFORMYLASE (EC 3.5.1.31) |
| 497 | 498 | RXS02308 | VV0127 | 13155 | 12727 | TRANSLATION INITIATION INHIBITOR |

Protein translocation, secretion, and folding

| | | | | | | |
|---|---|---|---|---|---|---|
| 499 | 500 | RXA01710 | GR00484 | 850 | 443 | PEPTIDE METHIONINE SULFOXIDE REDUCTASE |
| 501 | 502 | RXN02462 | VV0124 | 11932 | 13749 | PREPROTEIN TRANSLOCASE SECA SUBUNIT |
| 503 | 504 | F RXA00124 | GR00020 | 737 | 6 | PREPROTEIN TRANSLOCASE SECA SUBUNIT |
| 505 | 506 | F RXA02462 | GR00712 | 7653 | 6739 | PREPROTEIN TRANSLOCASE SECA SUBUNIT |
| 507 | 508 | RXA00125 | GR00020 | 1467 | 703 | PREPROTEIN TRANSLOCASE SECA SUBUNIT |
| 509 | 510 | RXA00687 | GR00179 | 9121 | 10440 | PREPROTEIN TRANSLOCASE SECY SUBUNIT |
| 511 | 512 | RXA02260 | GR00654 | 30280 | 30510 | PROTEIN-EXPORT MEMBRANE PROTEIN SECG HOMOLOG |
| 513 | 514 | RXN00046 | VV0119 | 5363 | 6058 | Signal recognition particle GTPase |
| 515 | 516 | F RXA00046 | GR00007 | 5363 | 6058 | Signal recognition particle GTPase |
| 517 | 518 | RXA00753 | GR00202 | 23301 | 21880 | PS1 PROTEIN PRECURSOR (PS1, one of the two major secreted proteins of |
| | | | | | | Corynebacterium glutamicum) |
| 519 | 520 | RXN03038 | VV0017 | 42941 | 43666 | PS1 PROTEIN PRECURSOR |
| 521 | 522 | F RXA01179 | GR00335 | 4639 | 5151 | PS1 PROTEIN PRECURSOR (PS1, one of the two major secreted proteins of |
| | | | | | | Corynebacterium glutamicum) |
| 523 | 524 | RXA01274 | GR00367 | 27148 | 28242 | PS1 PROTEIN PRECURSOR (PS1, one of the two major secreted proteins of |
| | | | | | | Corynebacterium glutamicum) |
| 525 | 526 | RXA01449 | GR00419 | 1046 | 6 | PS1 PROTEIN PRECURSOR (PS1, one of the two major secreted proteins of |
| | | | | | | Corynebacterium glutamicum) |
| 527 | 528 | RXA01798 | GR00509 | 276 | 4 | PS1 PROTEIN PRECURSOR (PS1, one of the two major secreted proteins of |
| | | | | | | Corynebacterium glutamicum) |
| 529 | 530 | RXA01818 | GR00515 | 6453 | 7439 | PS1 PROTEIN PRECURSOR (PS1, one of the two major secreted proteins of |
| | | | | | | Corynebacterium glutamicum) |
| 531 | 532 | RXA02607 | GR00742 | 13971 | 14189 | PS1 PROTEIN PRECURSOR (PS1, one of the two major secreted proteins of |
| | | | | | | Corynebacterium glutamicum) |
| 533 | 534 | RXA02608 | GR00742 | 14248 | 15942 | PS1 PROTEIN PRECURSOR (PS1, one of the two major secreted proteins of |
| | | | | | | Corynebacterium glutamicum) |
| 535 | 536 | RXN03054 | VV0026 | 1906 | 3486 | PS1 PROTEIN PRECURSOR |
| 537 | 538 | F RXA02886 | GR10021 | 1907 | 2737 | PS1 PROTEIN PRECURSOR (PS1, one of the two major secreted proteins of |
| | | | | | | Corynebacterium glutamicum) |
| 539 | 540 | RXN03039 | VV0018 | 2 | 631 | PS1 PROTEIN PRECURSOR |
| 541 | 542 | F RXA02894 | GR10036 | 1017 | 232 | PS1 PROTEIN PRECURSOR (PS1, one of the two major secreted proteins of |
| | | | | | | Corynebacterium glutamicum) |
| 543 | 544 | F RXA02904 | GR10042 | 686 | 12 | PS1 PROTEIN PRECURSOR (PS1, one of the two major secreted proteins of |
| | | | | | | Corynebacterium glutamicum) |
| 545 | 546 | RXA02025 | GR00614 | 862 | 212 | PEPTIDE METHIONINE SULFOXIDE REDUCTASE |
| 547 | 548 | RXA01431 | GR00417 | 7858 | 7538 | THIOREDOXIN REDUCTASE (EC 1.6.4.5)/THIOREDOXIN |
| 549 | 550 | RXA01432 | GR00417 | 8896 | 7946 | THIOREDOXIN REDUCTASE (EC 1.6.4.5) |
| 551 | 552 | RXN00937 | VV0079 | 42335 | 42706 | THIOREDOXIN |
| 553 | 554 | F RXA00937 | GR00256 | 1 | 123 | THIOREDOXIN |
| 555 | 556 | RXA01199 | GR00343 | 3813 | 4583 | THIOREDOXIN |
| 557 | 558 | RXA00824 | GR00221 | 4356 | 4913 | THIOL: DISULFIDE INTERCHANGE PROTEIN TLPA |
| 559 | 560 | RXA01841 | GR00522 | 115 | 477 | THIOL: DISULFIDE INTERCHANGE PROTEIN TLPA |
| 561 | 562 | RXN01863 | VV0206 | 1172 | 24 | /O/C Thioredoxin-like oxidoreductases |
| 563 | 564 | F RXA01863 | GR00530 | 830 | 24 | /O/C Thioredoxin-like oxidoreductases |
| 565 | 566 | RXA02323 | GR00668 | 1429 | 506 | THIOREDOXIN REDUCTASE (EC 1.6.4.5) |
| 567 | 568 | RXA01072 | GR00300 | 377 | 147 | NRDH-REDOXIN |
| 569 | 570 | RXA02436 | GR00709 | 1596 | 1036 | PEPTIDYL-PROLYL CIS-TRANS ISOMERASE (EC 5.2.1.8) |
| 571 | 572 | RXN01837 | VV0320 | 7103 | 7879 | PEPTIDYL-PROLYL CIS-TRANS ISOMERASE (EC 5.2.1.8) |
| 573 | 574 | F RXA01837 | GR00518 | 858 | 466 | PEPTIDYL-PROLYL CIS-TRANS ISOMERASE (EC 5.2.1.8) |
| 575 | 576 | RXS01277 | VV0009 | 32155 | 34158 | PROLYL ENDOPEPTIDASE (EC 3.4.21.26) |
| 577 | 578 | F RXA02047 | GR00624 | 1 | 192 | PROLYL ENDOPEPTIDASE (EC 3.4.21.26) |
| 579 | 580 | RXA02174 | GR00641 | 9290 | 8937 | PROBABLE FK506-BINDING PROTEIN (PEPTIDYL-PROLYL CIS-TRANS |
| | | | | | | ISOMERASE) (PPIASE) (EC 5.2.1.8) |
| 581 | 582 | RXA00568 | GR00152 | 2928 | 1582 | TRIGGER FACTOR |
| 583 | 584 | RXN03040 | VV0018 | 761 | 1069 | PS1 PROTEIN PRECURSOR |
| 585 | 586 | RXN03051 | VV0022 | 2832 | 3566 | PS1 PROTEIN PRECURSOR |
| 587 | 588 | RXN02949 | VV0025 | 31243 | 31575 | PREPROTEIN TRANSLOCASE SECE SUBUNIT |
| 589 | 590 | RXN00833 | VV0180 | 8039 | 8533 | THIOL PEROXIDASE (EC 1.11.1.—) |
| 591 | 592 | RXN01676 | VV0179 | 12059 | 11304 | THIOL: DISULFIDE INTERCHANGE PROTEIN DSBD |
| 593 | 594 | RXN00380 | VV0223 | 836 | 216 | THIOL: DISULFIDE INTERCHANGE PROTEIN TLPA |
| 595 | 596 | RXN02325 | VV0047 | 5527 | 6393 | THIOREDOXIN |
| 597 | 598 | RXN00493 | VV0086 | 14389 | 16002 | 60 KD CHAPERONIN |
| 599 | 600 | RXN02543 | VV0057 | 22031 | 20178 | DNAK PROTEIN |
| 601 | 602 | RXN01345 | VV0123 | 4883 | 3432 | Molecular chaperones (HSP70/DnaK family) |
| 603 | 604 | RXN02736 | VV0074 | 13600 | 14556 | PUTATIVE OXPPCYCLE PROTEIN OPCA |
| 605 | 606 | RXN02280 | VV0152 | 1849 | 26 | TRAP1 |
| 607 | 608 | RXS00170 | VV0031 | 4882 | 3029 | PS1 PROTEIN PRECURSOR |
| 609 | 610 | RXS02641 | VV0098 | 49070 | 51145 | PS1 PROTEIN PRECURSOR |
| 611 | 612 | RXS02650 | VV0090 | 6261 | 6839 | LIPOPROTEIN SIGNAL PEPTIDASE (EC 3.4.23.36) |
| 613 | 614 | RXS00076 | VV0154 | 2752 | 4122 | NADPH: FERREODOXIN OXIDOREDUCTASE PRECURSOR (EC 1.18.1.2) |
| 615 | 616 | RXS01438 | VV0089 | 25340 | 23976 | NADPH: FERREODOXIN OXIDOREDUCTASE PRECURSOR (EC 1.18.1.2) |

TABLE 2
GENES IDENTIFIED FROM GENBANK

| | | | |
|---|---|---|---|
| GenBank ™ | | | |
| Accession | | | |
| No. | Gene Name | Gene Function | Reference |

| | | | |
|---|---|---|---|
| A09073 | ppg | Phosphoenol pyruvate carboxylase | Bachmann, B. et al. “DNA fragment coding for phosphoenolpyruvat |
| | | | corboxylase, recombinant DNA carrying said fragment, strains carrying the |
| | | | recombinant DNA and method for producing L-aminino acids using said |
| | | | strains,” Patent: EP 0358940-A 3 Mar. 21, 1990 |
| A45579, | | Threonine dehydratase | Moeckel, B. et al. “Production of L-isoleucine by means of recombinant |
| A45581, | | | micro-organisms with deregulated threonine dehydratase,” Patent: WO |
| A45583, | | | 9519442-A 5 Jul. 20, 1995 |
| A45585 | | | |
| A45587 | | | |
| AB003132 | murC; ftsQ; | | Kobayashi, M. et al. “Cloning, sequencing, and characterization of the ftsZ |
| | ftsZ | | gene from coryneform bacteria,” Biochem. Biophys. Res. Commun., |
| | | | 236(2): 383-388 (1997) |
| AB015023 | murC; ftsQ | | Wachi, M. et al. “A murC gene from Coryneform bacteria,” Appl. Microbiol. |
| | | | Biotechnol., 51(2): 223-228 (1999) |
| AB018530 | dtsR | | Kimura, E. et al. “Molecular cloning of a novel gene, dtsR, which rescues the |
| | | | detergent sensitivity of a mutant derived from Brevibacterium |
| | | | lactofermentum,” Biosci. Biotechnol. Biochem., 60(10): 1565-1570 (1996) |
| AB018531 | dtsR1; dtsR2 | | |
| AB020624 | murI | D-glutamate racemase | |
| AB023377 | tkt | transketolase | |
| AB024708 | gltB; gltD | Glutamine 2-oxoglutarate | |
| | | aminotransferase | |
| | | large and small subunits | |
| AB025424 | acn | aconitase | |
| AB027714 | rep | Replication protein | |
| AB027715 | rep; aad | Replication protein; aminoglycoside | |
| | | adenyltransferase | |
| AF005242 | argC | N-acetylglutamate-5-semialdehyde | |
| | | dehydrogenase | |
| AF005635 | glnA | Glutamine synthetase | |
| AF030405 | hisF | cyclase | |
| AF030520 | argG | Argininosuccinate synthetase | |
| AF031518 | argF | Ornithine carbamolytransferase | |
| AF036932 | aroD | 3-dehydroquinate dehydratase | |
| AF038548 | pyc | Pyruvate carboxylase | |
| AF038651 | dciAE; apt; | Dipeptide-binding protein; adenine | Wehmeier, L. et al. “The role of the Corynebacterium glutamicum rel gene in |
| | rel | phosphoribosyltransferase; GTP | (p)ppGpp metabolism,” Microbiology, 144: 1853-1862 (1998) |
| | | pyrophosphokinase | |
| AF041436 | argR | Arginine repressor | |
| AF045998 | impA | Inositol monophosphate phosphatase | |
| AF048764 | argH | Argininosuccinate lyase | |
| AF049897 | argC; argJ; | N-acetylglutamylphosphate reductase; | |
| | argB; argD; | ornithine acetyltransferase; N- | |
| | argF; argR; | acetylglutamate kinase; acetylornithine | |
| | argG; argH | transminase; ornithine | |
| | | carbamoyltransferase; | |
| | | arginine repressor; | |
| | | argininosuccinate synthase; | |
| | | argininosuccinate lyase | |
| AF050109 | inhA | Enoyl-acyl carrier protein reductase | |
| AF050166 | hisG | ATP phosphoribosyltransferase | |
| AF051846 | hisA | Phosphoribosylformimino-5-amino-1- | |
| | | phosphoribosyl-4- | |
| | | imidazolecarboxamide | |
| | | isomerase | |
| AF052652 | metA | Homoserine O-acetyltransferase | Park, S. et al. “Isolation and analysis of metA, a methionine biosynthetic gene |
| | | | encoding homoserine acetyltransferase in Corynebacterium glutamicum,” Mol. |
| | | | Cells., 8(3): 286-294 (1998) |
| AF053071 | aroB | Dehydroquinate synthetase | |
| AF060558 | hisH | Glutamine amidotransferase | |
| AF086704 | hisE | Phosphoribosyl-ATP- | |
| | | pyrophosphohydrolase | |
| AF114233 | aroA | 5-enolpyruvylshikimate | |
| | | 3-phosphate synthase | |
| AF116184 | panD | L-aspartate-alpha-decarboxylase | Dusch, N. et al. “Expression of the Corynebacterium glutamicum panD gene |
| | | precursor | encoding L-aspartate-alpha-decarboxylase leads to pantothenate |
| | | | overproduction in Escherichia coli,” Appl. Environ. Microbiol., 65(4)1530-1539 |
| | | | (1999) |
| AF124518 | aroD; aroE | 3-dehydroquinase; shikimate | |
| | | dehydrogenase | |
| AF124600 | aroC; aroK; | Chorismate synthase; shikimate | |
| | aroB; pepQ | kinase; 3-dehydroquinate synthase; | |
| | | putative cytoplasmic peptidase | |
| AF145897 | inhA | | |
| AF145898 | inhA | | |
| AJ001436 | ectP | Transport of ectoine, glycine betaine, | Peter, H. et al. “Corynebacterium glutamicum is equipped with four secondary |
| | | proline | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| AJ004934 | dapD | Tetrahydrodipicolinate succinylase | Wehrmann, A. et al. “Different modes of diaminopimelate synthesis and their |
| | | (incompleteⁱ) | role in cell wall integrity: A study with Corynebacterium glutamicum,” J. |
| | | | Bacteriol., 180(12): 3159-3165 (1998) |
| AJ007732 | ppc; secG; | Phosphoenolpyruvate-carboxylase; ?; | |
| | amt; ocd; | high affinity ammonium uptake | |
| | soxA | protein; putative ornithine- | |
| | | cyclodecarboxylase; sarcosine | |
| | | oxidase | |
| AJ010319 | ftsY, glnB, | Involved in cell division; PII protein; | Jakoby, M. et al. “Nitrogen regulation in Corynebacterium glutamicum; |
| | glnD; srp; | uridylyltransferase (uridylyl-removing | Isolation of genes involved in biochemical characterization of corresponding |
| | amtP | enzmye); signal recognition | proteins,” FEMS Microbiol., 173(2): 303-310 (1999) |
| | | particle; low affinity ammonium | |
| | | uptake protein | |
| AJ132968 | cat | Chloramphenicol aceteyl transferase | |
| AJ224946 | mqo | L-malate: quinone oxidoreductase | Molenaar, D. et al. “Biochemical and genetic characterization of the |
| | | | membrane-associated malate dehydrogenase (acceptor) from Corynebacterium |
| | | | glutamicum,” Eur. J. Biochem., 254(2): 395-403 (1998) |
| AJ238250 | ndh | NADH dehydrogenase | |
| AJ238703 | porA | Porin | Lichtinger, T. et al. “Biochemical and biophysical characterization of the cell |
| | | | wall porin of Corynebacterium glutamicum: The channel is formed by a low |
| | | | molecular mass polypeptide,” Biochemistry, 37(43): 15024-15032 (1998) |
| D17429 | | Transposable element IS31831 | Vertes, A. A. et al. “Isolation and characterization of IS31831, a transposable |
| | | | element from Corynebacterium glutamicum,” Mol. Microbiol., 11(4): 739-746 |
| | | | (1994) |
| D84102 | odhA | 2-oxoglutarate dehydrogenase | Usuda, Y. et al. “Molecular cloning of the Corynebacterium glutamicum |
| | | | (Brevibacterium lactofermentum AJ12036) odhA gene encoding a novel type |
| | | | of 2-oxoglutarate dehydrogenase,” Microbiology, 142: 3347-3354 (1996) |
| E01358 | hdh; hk | Homoserine dehydrogenase; homoserine | Katsumata, R. et al. “Production of L-thereonine and L-isoleucine,” Patent: JP |
| | | kinase | 1987232392-A 1 Oct. 12, 1987 |
| E01359 | | Upstream of the start codon of | Katsumata, R. et al. “Production of L-thereonine and L-isoleucine,” Patent: JP |
| | | homoserine kinase gene | 1987232392-A 2 Oct. 12, 1987 |
| E01375 | | Tryptophan operon | |
| E01376 | trpL; trpE | Leader peptide; anthranilate synthase | Matsui, K. et al. “Tryptophan operon, peptide and protein coded thereby, |
| | | | utilization of tryptophan operon gene expression and production of |
| | | | tryptophan,” Patent: JP 1987244382-A 1 Oct. 24, 1987 |
| E01377 | | Promoter and operator regions of | Matsui, K. et al. “Tryptophan operon, peptide and protein coded thereby, |
| | | tryptophan operon | utilization of tryptophan operon gene expression and production of |
| | | | tryptophan,” Patent: JP 1987244382-A 1 Oct. 24, 1987 |
| E03937 | | Biotin-synthase | Hatakeyama, K. et al. “DNA fragment containing gene capable of coding |
| | | | biotin synthetase and its utilization,” Patent: JP 1992278088-A 1 Oct. 02, 1992 |
| E04040 | | Diamino pelargonic acid | Kohama, K. et al. “Gene coding diaminopelargonic acid aminotransferase and |
| | | aminotransferase | desthiobiotin synthetase and its utilization,” Patent: JP 1992330284-A 1 |
| | | | Nov. 18, 1992 |
| E04041 | | Desthiobiotinsynthetase | Kohama, K. et al. “Gene coding diaminopelargonic acid aminotransferase and |
| | | | desthiobiotin synthetase and its utilization,” Patent: JP 1992330284-A 1 |
| | | | Nov. 18, 1992 |
| E04307 | | Flavum aspartase | Kurusu, Y. et al. “Gene DNA coding aspartase and utilization thereof,” Patent: |
| | | | JP 1993030977-A 1 Feb. 09, 1993 |
| E04376 | | Isocitric acid lyase | Katsumata, R. et al. “Gene manifestation controlling DNA,” Patent: JP |
| | | | 1993056782-A 3 Mar. 09, 1993 |
| E04377 | | Isocitric acid lyase N-terminal fragment | Katsumata, R. et al. “Gene manifestation controlling DNA,” Patent: JP |
| | | | 1993056782-A 3 Mar. 09, 1993 |
| E04484 | | Prephenate dehydratase | Sotouchi, N. et al. “Production of L-phenylalanine by fermentation,” Patent: JP |
| | | | 1993076352-A 2 Mar. 30, 1993 |
| E05108 | | Aspartokinase | Fugono, N. et al. “Gene DNA coding Aspartokinase and its use,” Patent: JP |
| | | | 1993184366-A 1 Jul. 27, 1993 |
| E05112 | | Dihydro-dipichorinate synthetase | Hatakeyama, K. et al. “Gene DNA coding dihydrodipicolinic acid synthetase |
| | | | and its use,” Patent: JP 1993184371-A 1 Jul. 27, 1993 |
| E05776 | | Diaminopimelic acid dehydrogenase | Kobayashi, M. et al. “Gene DNA coding Diaminopimelic acid dehydrogenase |
| | | | and its use,” Patent: JP 1993284970-A 1 Nov. 02, 1993 |
| E05779 | | Threonine synthase | Kohama, K. et al. “Gene DNA coding threonine synthase and its use,” Patent: |
| | | | JP 1993284972-A 1 Nov. 02, 1993 |
| E06110 | | Prephenate dehydratase | Kikuchi, T. et al. “Production of L-phenylalanine by fermentation method,” |
| | | | Patent: JP 1993344881-A 1 Dec. 27, 1993 |
| E06111 | | Mutated Prephenate dehydratase | Kikuchi, T. et al. “Production of L-phenylalanine by fermentation method,” |
| | | | Patent: JP 1993344881-A 1 Dec. 27, 1993 |
| E06146 | | Acetohydroxy acid synthetase | Inui, M. et al. “Gene capable of coding Acetohydroxy acid synthetase and its |
| | | | use,” Patent: JP 1993344893-A 1 Dec. 27, 1993 |
| E06825 | | Aspartokinase | Sugimoto, M. et al. “Mutant aspartokinase gene,” patent: JP 1994062866-A 1 |
| | | | Mar. 08, 1994 |
| E06826 | | Mutated aspartokinase alpha subunit | Sugimoto, M. et al. “Mutant aspartokinase gene,” patent: JP 1994062866-A 1 |
| | | | Mar. 08, 1994 |
| E06827 | | Mutated aspartokinase alpha subunit | Sugimoto, M. et al. “Mutant aspartokinase gene,” patent: JP 1994062866-A 1 |
| | | | Mar. 08, 1994 |
| E07701 | secY | | Honno, N. et al. “Gene DNA participating in integration of membraneous |
| | | | protein to membrane,” Patent: JP 1994169780-A 1 Jun. 21, 1994 |
| E08177 | | Aspartokinase | Sato, Y. et al. “Genetic DNA capable of coding Aspartokinase released from |
| | | | feedback inhibition and its utilization,” Patent: JP 1994261766-A 1 Sep. 20, 1994 |
| E08178, | | Feedback inhibition-released | Sato, Y. et al. “Genetic DNA capable of coding Aspartokinase released from |
| E08179, | | Aspartokinase | feedback inhibition and its utilization,” Patent: JP 1994261766-A 1 Sep. 20, 1994 |
| E08180, | | | |
| E08181, | | | |
| E08182 | | | |
| E08232 | | Acetohydroxy-acid isomeroreductase | Inui, M. et al. “Gene DNA coding acetohydroxy acid isomeroreductase,” |
| | | | Patent: JP 1994277067-A 1 Oct. 04, 1994 |
| E08234 | secE | | Asai, Y. et al. “Gene DNA coding for translocation machinery of protein,” |
| | | | Patent: JP 1994277073-A 1 Oct. 04, 1994 |
| E08643 | | FT aminotransferase and desthiobiotin | Hatakeyama, K. et al. “DNA fragment having promoter function in |
| | | synthetase promoter region | coryneform bacterium,” Patent: JP 1995031476-A 1 Feb. 03, 1995 |
| E08646 | | Biotin synthetase | Hatakeyama, K. et al. “DNA fragment having promoter function in |
| | | | coryneform bacterium,” Patent: JP 1995031476-A 1 Feb. 03, 1995 |
| E08649 | | Aspartase | Kohama, K. et al “DNA fragment having promoter function in coryneform |
| | | | bacterium,” Patent: JP 1995031478-A 1 Feb. 03, 1995 |
| E08900 | | Dihydrodipicolinate reductase | Madori, M. et al. “DNA fragment containing gene coding Dihydrodipicolinate |
| | | | acid reductase and utilization thereof,” Patent: JP 1995075578-A 1 Mar. 20, 1995 |
| E08901 | | Diaminopimelic acid decarboxylase | Madori, M. et al. “DNA fragment containing gene coding Diaminopimelic acid |
| | | | decarboxylase and utilization thereof,” Patent: JP 1995075579-A 1 Mar. 20, 1995 |
| E12594 | | Serine hydroxymethyltransferase | Hatakeyama, K. et al. “Production of L-trypophan,” Patent: JP 1997028391-A |
| | | | 1 Feb. 04, 1997 |
| E12760, | | transposase | Moriya, M. et al. “Amplification of gene using artificial transposon,” Patent: |
| E12759, | | | JP 1997070291-A Mar. 18, 1997 |
| E12758 | | | |
| E12764 | | Arginyl-tRNA synthetase; | Moriya, M. et al. “Amplification of gene using artificial transposon,” Patent: |
| | | diaminopimelic acid decarboxylase | JP 1997070291-A Mar. 18, 1997 |
| E12767 | | Dihydrodipicolinic acid synthetase | Moriya, M. et al. “Amplification of gene using artificial transposon,” Patent: |
| | | | JP 1997070291-A Mar. 18, 1997 |
| E12770 | | aspartokinase | Moriya, M. et al. “Amplification of gene using artificial transposon,” Patent: |
| | | | JP 1997070291-A Mar. 18, 1997 |
| E12773 | | Dihydrodipicolinic acid reductase | Moriya, M. et al. “Amplification of gene using artificial transposon,” Patent: |
| | | | JP 1997070291-A Mar. 18, 1997 |
| E13655 | | Glucose-6-phosphate dehydrogenase | Hatakeyama, K. et al. “Glucose-6-phosphate dehydrogenase and DNA capable |
| | | | of coding the same,” Patent: JP 1997224661-A 1 Sep. 02, 1997 |
| L01508 | IlvA | Threonine dehydratase | Moeckel, B. et al. “Functional and structural analysis of the threonine |
| | | | dehydratase of Corynebacterium glutamicum,” J. Bacteriol., 174: 8065-8072 |
| | | | (1992) |
| L07603 | EC 4.2.1.15 | 3-deoxy-D-arabinoheptulosonate-7- | Chen, C. et al. “The cloning and nucleotide sequence of Corynebacterium |
| | | phosphate synthase | glutamicum 3-deoxy-D-arabinoheptulosonate-7-phosphate synthase gene,” |
| | | | FEMS Microbiol. Lett., 107: 223-230 (1993) |
| L09232 | IlvB; ilvN; | Acetohydroxy acid synthase large | Keilhauer, C. et al. “Isoleucine synthesis in Corynebacterium glutamicum: |
| | ilvC | subunit; Acetohydroxy acid synthase | molecular analysis of the ilvB-ilvN-ilvC operon,” J. Bacteriol., 175(17): |
| | | small subunit; Acetohydroxy acid | 5595-5603 (1993) |
| | | isomeroreductase | |
| L18874 | PtsM | Phosphoenolpyruvate sugar | Fouet, A et al. “Bacillus subtilis sucrose-specific enzyme II of the |
| | | phosphotransferase | phosphotransferase system: expression in Escherichia coli and homology to |
| | | | enzymes II from enteric bacteria,” PNAS USA, 84(24): 8773-8777 (1987); |
| | | | Lee, J. K. et al. “Nucleotide sequence of the gene encoding the |
| | | | Corynebacterium glutamicum mannose enzyme II and analyses of |
| | | | the deduced protein sequence,” FEMS Microbiol. Lett., |
| | | | 119(1-2): 137-145 (1994) |
| L27123 | aceB | Malate synthase | Lee, H-S. et al. “Molecular characterization of aceB, a gene encoding malate |
| | | | synthase in Corynebacterium glutamicum,” J. Microbiol. Biotechnol., |
| | | | 4(4): 256-263 (1994) |
| L27126 | | Pyruvate kinase | Jetten, M. S. et al. “Structural and functional analysis of pyruvate kinase from |
| | | | Corynebacterium glutamicum,” Appl. Environ. Microbiol., 60(7): 2501-2507 |
| | | | (1994) |
| L28760 | aceA | Isocitrate lyase | |
| L35906 | dtxr | Diphtheria toxin repressor | Oguiza, J. A. et al. “Molecular cloning, DNA sequence analysis, and |
| | | | characterization of the Corynebacterium diphtheriae dtxR from Brevibacterium |
| | | | lactofermentum,” J. Bacteriol., 177(2): 465-467 (1995) |
| M13774 | | Prephenate dehydratase | Follettie, M. T. et al. “Molecular cloning and nucleotide sequence of the |
| | | | Corynebacterium glutamicum pheA gene,” J. Bacteriol., 167: 695-702 (1986) |
| M16175 | 5S rRNA | | Park, Y-H. et al. “Phylogenetic analysis of the coryneform bacteria by 56 |
| | | | rRNA sequences,” J. Bacteriol., 169: 1801-1806 (1987) |
| M16663 | trpE | Anthranilate synthase, 5′ end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M16664 | trpA | Tryptophan synthase, 3′end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M25819 | | Phosphoenolpyruvate carboxylase | O'Regan, M. et al. “Cloning and nucleotide sequence of the |
| | | | Phosphoenolpyruvate carboxylase-coding gene of Corynebacterium |
| | | | glutamicum ATCC13032,” Gene, 77(2): 237-251 (1989) |
| M85106 | | 23S rRNA gene insertion sequence | Roller, C. et al. “Gram-positive bacteria with a high DNA G + C content are |
| | | | characterized by a common insertion within their 23S rRNA genes,” J. Gen. |
| | | | Microbiol., 138: 1167-1175 (1992) |
| M85107, | | 23S rRNA gene insertion sequence | Roller, C. et al. “Gram-positive bacteria with a high DNA G + C content are |
| M85108 | | | characterized by a common insertion within their 23S rRNA genes,” J. Gen. |
| | | | Microbiol., 138: 1167-1175 (1992) |
| M89931 | aecD; brnQ; | Beta C-S lyase; branched-chain amino | Rossol, I. et al. “The Corynebacterium glutamicum aecD gene encodes a C-S |
| | yhbw | acid uptake carrier; hypothetical | lyase with alpha, beta-elimination activity that degrades aminoethylcysteine,” |
| | | protein yhbw | J. Bacteriol., 174(9): 2968-2977 (1992); Tauch, A. et al. “Isoleucine uptake in |
| | | | Corynebacterium glutamicum ATCC 13032 is directed by the brnQ gene |
| | | | product,” Arch. Microbiol., 169(4): 303-312 (1998) |
| S59299 | trp | Leader gene (promoter) | Herry, D. M. et al. “Cloning of the trp gene cluster from a tryptophan- |
| | | | hyperproducing strain of Corynebacterium glutamicum: identification of a |
| | | | mutation in the trp leader sequence,” Appl. Environ. Microbiol., 59(3): 791-799 |
| | | | (1993) |
| U11545 | trpD | Anthranilate phosphoribosyltransferase | O'Gara, J. P. and Dunican, L. K. (1994) Complete nucleotide sequence of the |
| | | | Corynebacterium glutamicum ATCC 21850 tpD gene.” Thesis, Microbiology |
| | | | Department, University College Galway, Ireland. |
| U13922 | cglIM; | Putative type II 5-cytosoine | Schafer, A. et al. “Cloning and characterization of a DNA region encoding a |
| | cglIR; | methyltransferase; putative type II | stress-sensitive restriction system from Corynebacterium glutamicum ATCC |
| | clgIIR | restriction endonuclease; putative | 13032 and analysis of its role in intergeneric conjugation with Escherichia |
| | | type I or type III restriction | coli,” J. Bacteriol., 176(23): 7309-7319 (1994); Schafer, A. et al. “The |
| | | | Corynebacterium glutamicum cglIM gene encoding a 5-cytosine in an McrBC- |
| | | | deficient Escherichia coli strain,” Gene, 203(2): 95-101 (1997) |
| U14965 | recA | | |
| U31224 | ppx | | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31225 | proC | L-proline: NADP+ 5-oxidoreductase | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31230 | obg; proB; | ?; gamma glutamyl kinase; similar to D- | Ankri, S. et al. “Mutations in the Corynebacterium glutamicumproline |
| | unkdh | isomer specific 2-hydroxyacid | biosynthetic pathway: A natural bypass of the proA step,” J. Bacteriol., |
| | | dehydrogenases | 178(15): 4412-4419 (1996) |
| X54223 | | AttB-related site | Cianciotto, N. et al. “DNA sequence homology between att B-related sites of |
| | | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X54740 | argS; lysA | Arginyl-tRNA synthetase; | Marcel, T. et al. “Nucleotide sequence and organization of the upstream region |
| | | Diaminopimelate decarboxylase | of the Corynebacterium glutamicum lysA gene,” Mol. Microbiol., 4(11): |
| | | | 1819-1830 (1990) |
| X55994 | trpL; trpE | Putative leader peptide; anthranilate | Heery, D. M. et al. “Nucleotide sequence of the Corynebacterium glutamicum |
| | | synthase component 1 | trpE gene,” Nucleic Acids Res., 18(23): 7138 (1990) |
| X56037 | thrC | Threonine synthase | Han, K. S. et al. “The molecular structure of the Corynebacterium glutamicum |
| | | | threonine synthase gene,” Mol. Microbiol., 4(10): 1693-1702 (1990) |
| X56075 | attB-related | Attachment site | Cianciotto, N. et al. “DNA sequence homology between att B-related sites of |
| | site | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X57226 | lysC-alpha; | Aspartokinase-alpha subunit; | Kalinowski, J. et al. “Genetic and biochemical analysis of the Aspartokinase |
| | lysC-beta; | Aspartokinase-beta subunit; aspartate | from Corynebacterium glutamicum,” Mol. Microbiol., 5(5): 1197-1204 (1991); |
| | asd | beta semialdehyde dehydrogenase | Kalinowski, J. et al. “Aspartokinase genes lysC alpha and lysC beta overlap |
| | | | and are adjacent to the aspertate beta-semialdehyde dehydrogenase gene asd in |
| | | | Corynebacterium glutamicum,” Mol. Gen. Genet., 224(3): 317-324 (1990) |
| X59403 | gap; pgk; tpi | Glyceraldehyde-3-phosphate; | Eikmanns, B. J. “Identification, sequence analysis, and expression of a |
| | | phosphoglycerate kinase; | Corynebacterium glutamicum gene cluster encoding the three glycolytic |
| | | triosephosphate isomerase | enzymes glyceraldehyde-3-phosphate dehydrogenase, 3-phosphoglycerate |
| | | | kinase, and triosephosphate isomeras,” J. Bacteriol., 174(19): 6076-6086 |
| | | | (1992) |
| X59404 | gdh | Glutamate dehydrogenase | Bormann, E. R. et al. “Molecular analysis of the Corynebacterium glutamicum |
| | | | gdh gene encoding glutamate dehydrogenase,” Mol. Microbiol., 6(3): 317-326 |
| | | | (1992) |
| X60312 | lysI | L-lysine permease | Seep-Feldhaus, A. H. et al. “Molecular analysis of the Corynebacterium |
| | | | glutamicum lysl gene involved in lysine uptake,” Mol. Microbiol., 5(12): |
| | | | 2995-3005 (1991) |
| X66078 | cop1 | Ps1 protein | Joliff, G. et al. “Cloning and nucleotide sequence of the csp1 gene encoding |
| | | | PS1, one of the two major secreted proteins of Corynebacterium glutamicum: |
| | | | The deduced N-terminal region of PS1 is similar to the Mycobacterium antigen |
| | | | 85 complex,” Mol. Microbiol., 6(16): 2349-2362 (1992) |
| X66112 | glt | Citrate synthase | Eikmanns, B. J. et al. “Cloning sequence, expression and transcriptional |
| | | | analysis of the Corynebacterium glutamicum gltA gene encoding citrate |
| | | | synthase,” Microbiol., 140: 1817-1828 (1994) |
| X67737 | dapB | Dihydrodipicolinate reductase | |
| X69103 | csp2 | Surface layer protein PS2 | Peyret, J. L. et al. “Characterization of the cspB gene encoding PS2, an ordered |
| | | | surface-layer protein in Corynebacterium glutamicum,” Mol. Microbiol., |
| | | | 9(1): 97-109 (1993) |
| X69104 | | IS3 related insertion element | Bonamy, C. et al. “Identification of IS1206, a Corynebacterium glutamicum |
| | | | IS3-related insertion sequence and phylogenetic analysis,” Mol. Microbiol., |
| | | | 14(3): 571-581 (1994) |
| X70959 | leuA | Isopropylmalate synthase | Patek, M. et al. “Leucine synthesis in Corynebacterium glutamicum: enzyme |
| | | | activities, structure of leuA, and effect of leuA inactivation on lysine |
| | | | synthesis,” Appl. Environ. Microbiol., 60(1): 133-140 (1994) |
| X71489 | icd | Isocitrate dehydrogenase (NADP+) | Eikmanns, B. J. et al. “Cloning sequence analysis, expression, and inactivation |
| | | | of the Corynebacterium glutamicum icd gene encoding isocitrate |
| | | | dehydrogenase and biochemical characterization of the enzyme,” J. Bacteriol., |
| | | | 177(3): 774-782 (1995) |
| X72855 | GDHA | Glutamate dehydrogenase (NADP+) | |
| X75083, | mtrA | 5-methyltryptophan resistance | Heery, D. M. et al. “A sequence from a tryptophan-hyperproducing strain of |
| X70584 | | | Corynebacterium glutamicum encoding resistance to 5-methyltryptophan,” |
| | | | Biochem. Biophys. Res. Commun., 201(3): 1255-1262 (1994) |
| X75085 | recA | | Fitzpatrick, R. et al. “Construction and characterization of recA mutant strains |
| | | | of Corynebacterium glutamicum and Brevibacterium lactofermentum,” Appl. |
| | | | Microbiol. Biotechnol., 42(4): 575-580 (1994) |
| X75504 | aceA; thiX | Partial Isocitrate lyase; ? | Reinscheid, D. J. et al. “Characterization of the isocitrate lyase gene from |
| | | | Corynebacterium glutamicum and biochemical analysis of the enzyme,” J. |
| | | | Bacteriol., 176(12): 3474-3483 (1994) |
| X76875 | | ATPase beta-subunit | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77034 | tuf | Elongation factor Tu | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77384 | recA | | Billman-Jacobe, H. “Nucleotide sequence of a recA gene from |
| | | | Corynebacterium glutamicum,” DNA Seq., 4(6): 403-404 (1994) |
| X78491 | aceB | Malate synthase | Reinscheid, D. J. et al. “Malate synthase from Corynebacterium glutamicum |
| | | | pta-ack operon encoding phosphotransacetylase: sequence analysis,” |
| | | | Microbiology, 140: 3099-3108 (1994) |
| X80629 | 16S rDNA | 16S ribosomal RNA | Rainey, F. A. et al. “Phylogenetic analysis of the genera Rhodococcus and |
| | | | Norcardia and evidence for the evolutionary origin of the genus Norcardia |
| | | | from within the radiation of Rhodococcus species,” Microbiol., 141: 523-528 |
| | | | (1995) |
| X81191 | gluA; gluB; | Glutamate uptake system | Kronemeyer, W. et al. “Structure of the gluABCD cluster encoding the |
| | gluC; gluD | | glutamate uptake system of Corynebacterium glutamicum,” J. Bacteriol., |
| | | | 177(5): 1152-1158 (1995) |
| X81379 | dapE | Succinyldiaminopimelate desuccinylase | Wehrmann, A. et al. “Analysis of different DNA fragments of |
| | | | Corynebacterium glutamicum complementing dapE of Escherichia coli,” |
| | | | Microbiology, 40: 3349-56 (1994) |
| X82061 | 16S rDNA | 16S ribosomal RNA | Ruimy, R. et al. “Phylogeny of the genus Corynebacterium deduced from |
| | | | analyses of small-subunit ribosomal DNA sequences,” Int. J. Syst. Bacteriol., |
| | | | 45(4): 740-746 (1995) |
| X82928 | asd; lysC | Aspartate-semialdehyde | Serebrijski, I. et al. “Multicopy suppression by asd gene and osmotic stress- |
| | | dehydrogenase; ? | dependent complementation by heterologous proA in proA mutants,” J. |
| | | | Bacteriol., 177(24): 7255-7260 (1995) |
| X82929 | proA | Gamma-glutamyl phosphate reductase | Serebrijski, I. et al. “Multicopy suppression by asd gene and osmotic stress- |
| | | | dependent complementation by heterologous proA in proA mutants,” J. |
| | | | Bacteriol., 177(24): 7255-7260 (1995) |
| X84257 | 16S rDNA | 16S ribosomal RNA | Pascual, C. et al. “Phylogenetic analysis of the genus Corynebacterium based |
| | | | on 16S rRNA gene sequences,” Int. J. Syst. Bacteriol., 45(4): 724-728 (1995) |
| X85965 | aroP; dapE | Aromatic amino acid permease; ? | Wehrmann, A. et al. “Functional analysis of sequences adjacent to dapE of |
| | | | Corynebacterium glutamicumproline reveals the presence of aroP, which |
| | | | encodes the aromatic amino acid transporter,” J. Bacteriol., 177(20): 5991-5993 |
| | | | (1995) |
| X86157 | argB; argC; | Acetylglutamate kinase; N-acetyl- | Sakanyan, V. et al. “Genes and enzymes of the acetyl cycle of arginine |
| | argD; argF; | gamma-glutamyl-phosphate | biosynthesis in Corynebacterium glutamicum: enzyme evolution in the early |
| | argJ | reductase; acetylornithine | steps of the arginine pathway,” Microbiology, 142: 99-108 (1996) |
| | | aminotransferase; ornithine | |
| | | carbamoyltransferase; | |
| | | glutamate N-acetyltransferase | |
| X89084 | pta; ackA | Phosphate acetyltransferase; | Reinscheid, D. J. et al. “Cloning, sequence analysis, expression and inactivation |
| | | acetate kinase | of the Corynebacterium glutamicum pta-ack operon encoding |
| | | | phosphotransacetylase and acetate kinase,” Microbiology, 145: 503-513 (1999) |
| X89850 | attB | Attachment site | Le Marrec, C. et al. “Genetic characterization of site-specific integration |
| | | | functions of phi AAU2 infecting “Arthrobacter aureus C70,” J. Bacteriol., |
| | | | 178(7): 1996-2004 (1996) |
| X90356 | | Promoter fragment F1 | Patek, M. et al. “Promoters from Corynebacterium glutamicum: cloning, |
| | | | molecular analysis and search for a consensus motif,” Microbiology, |
| | | | 142: 1297-1309 (1996) |
| X90357 | | Promoter fragment F2 | Patek, M. et al. “Promoters from Corynebacterium glutamicum: cloning, |
| | | | molecular analysis and search for a consensus motif,” Microbiology, |
| | | | 142: 1297-1309 (1996) |
| X90358 | | Promoter fragment F10 | Patek, M. et al. “Promoters from Corynebacterium glutamicum: cloning, |
| | | | molecular analysis and search for a consensus motif,” Microbiology, |
| | | | 142: 1297-1309 (1996) |
| X90359 | | Promoter fragment F13 | Patek, M. et al. “Promoters from Corynebacterium glutamicum: cloning, |
| | | | molecular analysis and search for a consensus motif,” Microbiology, |
| | | | 142: 1297-1309 (1996) |
| X90360 | | Promoter fragment F22 | Patek, M. et al. “Promoters from Corynebacterium glutamicum: cloning, |
| | | | molecular analysis and search for a consensus motif,” Microbiology, |
| | | | 142: 1297-1309 (1996) |
| X90361 | | Promoter fragment F34 | Patek, M. et al. “Promoters from Corynebacterium glutamicum: cloning, |
| | | | molecular analysis and search for a consensus motif,” Microbiology, |
| | | | 142: 1297-1309 (1996) |
| X90362 | | Promoter fragment F37 | Patek, M. et al. “Promoters from Corynebacterium glutamicum: cloning, |
| | | | molecular analysis and search for a consensus motif,” Microbiology, |
| | | | 142: 1297-1309 (1996) |
| X90363 | | Promoter fragment F45 | Patek, M. et al. “Promoters from Corynebacterium glutamicum: cloning, |
| | | | molecular analysis and search for a consensus motif,” Microbiology, |
| | | | 142: 1297-1309 (1996) |
| X90364 | | Promoter fragment F64 | Patek, M. et al. “Promoters from Corynebacterium glutamicum: cloning, |
| | | | molecular analysis and search for a consensus motif,” Microbiology, |
| | | | 142: 1297-1309 (1996) |
| X90365 | | Promoter fragment F75 | Patek, M. et al. “Promoters from Corynebacterium glutamicum: cloning, |
| | | | molecular analysis and search for a consensus motif,” Microbiology, |
| | | | 142: 1297-1309 (1996) |
| X90366 | | Promoter fragment PF101 | Patek, M. et al. “Promoters from Corynebacterium glutamicum: cloning, |
| | | | molecular analysis and search for a consensus motif,” Microbiology, |
| | | | 142: 1297-1309 (1996) |
| X90367 | | Promoter fragment PF104 | Patek, M. et al. “Promoters from Corynebacterium glutamicum: cloning, |
| | | | molecular analysis and search for a consensus motif,” Microbiology, |
| | | | 142: 1297-1309 (1996) |
| X90368 | | Promoter fragment PF109 | Patek, M. et al. “Promoters from Corynebacterium glutamicum: cloning, |
| | | | molecular analysis and search for a consensus motif,” Microbiology, |
| | | | 142: 1297-1309 (1996) |
| X93513 | amt | Ammonium transport system | Siewe, R. M. et al. “Functional and genetic characterization of the (methyl) |
| | | | ammonium uptake carrier of Corynebacterium glutamicum,” J. Biol. Chem., |
| | | | 271(10): 5398-5403 (1996) |
| X93514 | betP | Glycine betaine transport system | Peter, H. et al. “Isolation, characterization, and expression of the |
| | | | Corynebacterium glutamicum betP gene, encoding the transport system for the |
| | | | compatible solute glycine betaine,” J. Bacteriol., 178(17): 5229-5234 (1996) |
| X95649 | orf4 | | Patek, M. et al. “Identification and transcriptional analysis of the dapB-ORF2- |
| | | | dapA-ORF4 operon of Corynebacterium glutamicum, encoding two enzymes |
| | | | involved in L-lysine synthesis,” Biotechnol. Lett., 19: 1113-1117 (1997) |
| X96471 | lysE; lysG | Lysine exporter protein; Lysine export | Vrljic, M. et al. “A new type of transporter with a new type of cellular |
| | | regulator protein | function: L-lysine export from Corynebacterium glutamicum,” Mol. |
| | | | Microbiol., 22(5): 815-826 (1996) |
| X96580 | panB; panC; | 3-methyl-2-oxobutanoate | Sahm, H. et al. “D-pantothenate synthesis in Corynebacterium glutamicum and |
| | xylB | hydroxymethyltransferase; pantoate- | use of panBC and genes encoding L-valine synthesis for D-pantothenate |
| | | beta-alanine ligase; xylulokinase | overproduction,” Appl. Environ. Microbiol., 65(5): 1973-1979 (1999) |
| X96962 | | Insertion sequence IS1207 and | |
| | | transposase | |
| X99289 | | Elongation factor P | Ramos, A. et al. “Cloning, sequencing and expression of the gene encoding |
| | | | elongation factor P in the amino-acid producer Brevibacterium lactofermentum |
| | | | (Corynebacterium glutamicum ATCC 13869),” Gene, 198: 217-222 (1997) |
| Y00140 | thrB | Homoserine kinase | Mateos, L. M. et al. “Nucleotide sequence of the homoserine kinase (thrB) gene |
| | | | of the Brevibacterium lactofermentum,” Nucleic Acids Res., 15(9): 3922 (1987) |
| Y00151 | ddh | Meso-diaminopimelate | Ishino, S. et al. “Nucleotide sequence of the meso-diaminopimelate D- |
| | | D-dehydrogenase (EC 1.4.1.16) | dehydrogenase gene from Corynebacterium glutamicum,” Nucleic Acids Res., |
| | | | 15(9): 3917 (1987) |
| Y00476 | thrA | Homoserine dehydrogenase | Mateos, L. M. et al. “Nucleotide sequence of the homoserine dehydrogenase |
| | | | (thrA) gene of the Brevibacterium lactofermentum,” Nucleic Acids Res., |
| | | | 15(24): 10598 (1987) |
| Y00546 | hom; thrB | Homoserine dehydrogenase; homoserine | Peoples, O. P. et al. “Nucleotide sequence and fine structural analysis of the |
| | | kinase | Corynebacterium glutamicum hom-thrB operon,” Mol. Microbiol., 2(1): 63-72 |
| | | | (1988) |
| Y08964 | murC; ftsQ/ | UPD-N-acetylmuramate-alanine ligase; | Honrubia, M. P. et al. “Identification, characterization, and chromosomal |
| | divD; ftsZ | division initiation protein or cell | organization of the ftsZ gene from Brevibacterium lactofermentum,” Mol. Gen. |
| | | division protein; cell division | Genet., 259(1): 97-104 (1998) |
| | | protein | |
| Y09163 | putP | High affinity proline transport system | Peter, H. et al. “Isolation of the putP gene of Corynebacterium |
| | | | glutamicumproline and characterization of a low-affinity uptake system for |
| | | | compatible solutes,” Arch. Microbiol., 168(2): 143-151 (1997) |
| Y09548 | pyc | Pyruvate carboxylase | Peters-Wendisch, P. G. et al. “Pyruvate carboxylase from Corynebacterium |
| | | | glutamicum: characterization, expression and inactivation of the pyc gene,” |
| | | | Microbiology, 144: 915-927 (1998) |
| Y09578 | leuB | 3-isopropylmalate dehydrogenase | Patek, M. et al. “Analysis of the leuB gene from Corynebacterium |
| | | | glutamicum,” Appl. Microbiol. Biotechnol., 50(1): 42-47 (1998) |
| Y12472 | | Attachment site bacteriophage Phi-16 | Moreau, S. et al. “Site-specific integration of corynephage Phi-16: The |
| | | | construction of an integration vector,” Microbiol., 145: 539-548 (1999) |
| Y12537 | proP | Proline/ectoine uptake system protein | Peter, H. et al. “Corynebacterium glutamicum is equipped with four secondary |
| | | | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| Y13221 | glnA | Glutamine synthetase I | Jakoby, M. et al. “Isolation of Corynebacterium glutamicum glnA gene |
| | | | encoding glutamine synthetase I,” FEMS Microbiol. Lett., 154(1): 81-88 (1997) |
| Y16642 | lpd | Dihydrolipoamide dehydrogenase | |
| Y18059 | | Attachment site Corynephage 304L | Moreau, S. et al. “Analysis of the integration functions of &phi; 304L: An |
| | | | integrase module among corynephages,” Virology, 255(1): 150-159 (1999) |
| Z21501 | argS; lysA | Arginyl-tRNA synthetase; | Oguiza, J. A. et al. “A gene encoding arginyl-tRNA synthetase is located in the |
| | | diaminopimelate decarboxylase | upstream region of the lysA gene in Brevibacterium lactofermentum: |
| | | (partial) | Regulation of argS-lysA cluster expression by arginine,” J. |
| | | | Bacteriol., 175(22): 7356-7362 (1993) |
| Z21502 | dapA; dapB | Dihydrodipicolinate synthase; | Pisabarro, A. et al. “A cluster of three genes (dapA, orf2, and dapB) of |
| | | dihydrodipicolinate reductase | Brevibacterium lactofermentum encodes dihydrodipicolinate reductase, and a |
| | | | third polypeptide of unknown function,” J. Bacteriol., 175(9): 2743-2749 |
| | | | (1993) |
| Z29563 | thrC | Threonine synthase | Malumbres, M. et al. “Analysis and expression of the thrC gene of the encoded |
| | | | threonine synthase,” Appl. Environ. Microbiol., 60(7)2209-2219 (1994) |
| Z46753 | 16S rDNA | Gene for 16S ribosomal RNA | |
| Z49822 | sigA | SigA sigma factor | Oguiza, J. A. et al “Multiple sigma factor genes in Brevibacterium |
| | | | lactofermentum: Characterization of sigA and sigB,” J. Bacteriol., 178(2): |
| | | | 550-553 (1996) |
| Z49823 | galE; dtxR | Catalytic activity UDP-galactose 4- | Oguiza, J. A. et al “The galE gene encoding the UDP-galactose 4-epimerase of |
| | | epimerase; diphtheria toxin regulatory | Brevibacterium lactofermentum is coupled transcriptionally to the dmdR |
| | | protein | gene,” Gene, 177: 103-107 (1996) |
| Z49824 | orfl; sigB | ?; SigB sigma factor | Oguiza, J. A. et al “Multiple sigma factor genes in Brevibacterium |
| | | | lactofermentum: Characterization of sigA and sigB,” J. Bacteriol., 178(2): |
| | | | 550-553 (1996) |
| Z66534 | | Transposase | Correia, A. et al. “Cloning and characterization of an IS-like element present in |
| | | | the genome of Brevibacterium lactofermentum ATCC 13869,” Gene, |
| | | | 170(1): 91-94 (1996) |

¹A sequence for this gene was published in the indicated reference. However, the sequence obtained by the inventors of the present application is significantly longer than the published version. It is believed that the published version relied on an incorrect start codon, and thus represents only a fragment of the actual coding region.

TABLE 3
Corynebacterium and Brevibacterium Strains Which May be Used in the Practice of the Invention

| Genus | species | ATCC | FERM | NRRL | CECT | NCIMB | CBS | NCTC | DSMZ |
|---|---|---|---|---|---|---|---|---|---|
| Brevibacterium | ammoniagenes | 21054 | | | | | | | |
| Brevibacterium | ammoniagenes | 19350 | | | | | | | |
| Brevibacterium | ammoniagenes | 19351 | | | | | | | |
| Brevibacterium | ammoniagenes | 19352 | | | | | | | |
| Brevibacterium | ammoniagenes | 19353 | | | | | | | |
| Brevibacterium | ammoniagenes | 19354 | | | | | | | |
| Brevibacterium | ammoniagenes | 19355 | | | | | | | |
| Brevibacterium | ammoniagenes | 19356 | | | | | | | |
| Brevibacterium | ammoniagenes | 21055 | | | | | | | |
| Brevibacterium | ammoniagenes | 21077 | | | | | | | |
| Brevibacterium | ammoniagenes | 21553 | | | | | | | |
| Brevibacterium | ammoniagenes | 21580 | | | | | | | |
| Brevibacterium | ammoniagenes | 39101 | | | | | | | |
| Brevibacterium | butanicum | 21196 | | | | | | | |
| Brevibacterium | divaricatum | 21792 | P928 | | | | | | |
| Brevibacterium | flavum | 21474 | | | | | | | |
| Brevibacterium | flavum | 21129 | | | | | | | |
| Brevibacterium | flavum | 21518 | | | | | | | |
| Brevibacterium | flavum | | | B11474 | | | | | |
| Brevibacterium | flavum | | | B11472 | | | | | |
| Brevibacterium | flavum | 21127 | | | | | | | |
| Brevibacterium | flavum | 21128 | | | | | | | |
| Brevibacterium | flavum | 21427 | | | | | | | |
| Brevibacterium | flavum | 21475 | | | | | | | |
| Brevibacterium | flavum | 21517 | | | | | | | |
| Brevibacterium | flavum | 21528 | | | | | | | |
| Brevibacterium | flavum | 21529 | | | | | | | |
| Brevibacterium | flavum | | | B11477 | | | | | |
| Brevibacterium | flavum | | | B11478 | | | | | |
| Brevibacterium | flavum | 21127 | | | | | | | |
| Brevibacterium | flavum | | | B11474 | | | | | |
| Brevibacterium | healii | 15527 | | | | | | | |
| Brevibacterium | ketoglutamicum | 21004 | | | | | | | |
| Brevibacterium | ketoglutamicum | 21089 | | | | | | | |
| Brevibacterium | ketosoreductum | 21914 | | | | | | | |
| Brevibacterium | lactofermentum | | | | 70 | | | | |
| Brevibacterium | lactofermentum | | | | 74 | | | | |
| Brevibacterium | lactofermentum | | | | 77 | | | | |
| Brevibacterium | lactofermentum | 21798 | | | | | | | |
| Brevibacterium | lactofermentum | 21799 | | | | | | | |
| Brevibacterium | lactofermentum | 21800 | | | | | | | |
| Brevibacterium | lactofermentum | 21801 | | | | | | | |
| Brevibacterium | lactofermentum | | | B11470 | | | | | |
| Brevibacterium | lactofermentum | | | B11471 | | | | | |
| Brevibacterium | lactofermentum | 21086 | | | | | | | |
| Brevibacterium | lactofermentum | 21420 | | | | | | | |
| Brevibacterium | lactofermentum | 21086 | | | | | | | |
| Brevibacterium | lactofermentum | 31269 | | | | | | | |
| Brevibacterium | linens | 9174 | | | | | | | |
| Brevibacterium | linens | 19391 | | | | | | | |
| Brevibacterium | linens | 8377 | | | | | | | |
| Brevibacterium | paraffinolyticum | | | | | 11160 | | | |
| Brevibacterium | spec. | | | | | | 717.73 | | |
| Brevibacterium | spec. | | | | | | 717.73 | | |
| Brevibacterium | spec. | 14604 | | | | | | | |
| Brevibacterium | spec. | 21860 | | | | | | | |
| Brevibacterium | spec. | 21864 | | | | | | | |
| Brevibacterium | spec. | 21865 | | | | | | | |
| Brevibacterium | spec. | 21866 | | | | | | | |
| Brevibacterium | spec. | 19240 | | | | | | | |
| Corynebacterium | acetoacidophilum | 21476 | | | | | | | |
| Corynebacterium | acetoacidophilum | 13870 | | | | | | | |
| Corynebacterium | acetoglutamicum | | | B11473 | | | | | |
| Corynebacterium | acetoglutamicum | | | B11475 | | | | | |
| Corynebacterium | acetoglutamicum | 15806 | | | | | | | |
| Corynebacterium | acetoglutamicum | 21491 | | | | | | | |
| Corynebacterium | acetoglutamicum | 31270 | | | | | | | |
| Corynebacterium | acetophilum | | | B3671 | | | | | |
| Corynebacterium | ammoniagenes | 6872 | | | | | | 2399 | |
| Corynebacterium | ammoniagenes | 15511 | | | | | | | |
| Corynebacterium | fujiokense | 21496 | | | | | | | |
| Corynebacterium | glutamicum | 14067 | | | | | | | |
| Corynebacterium | glutamicum | 39137 | | | | | | | |
| Corynebacterium | glutamicum | 21254 | | | | | | | |
| Corynebacterium | glutamicum | 21255 | | | | | | | |
| Corynebacterium | glutamicum | 31830 | | | | | | | |
| Corynebacterium | glutamicum | 13032 | | | | | | | |
| Corynebacterium | glutamicum | 14305 | | | | | | | |
| Corynebacterium | glutamicum | 15455 | | | | | | | |
| Corynebacterium | glutamicum | 13058 | | | | | | | |
| Corynebacterium | glutamicum | 13059 | | | | | | | |
| Corynebacterium | glutamicum | 13060 | | | | | | | |
| Corynebacterium | glutamicum | 21492 | | | | | | | |
| Corynebacterium | glutamicum | 21513 | | | | | | | |
| Corynebacterium | glutamicum | 21526 | | | | | | | |
| Corynebacterium | glutamicum | 21543 | | | | | | | |
| Corynebacterium | glutamicum | 13287 | | | | | | | |
| Corynebacterium | glutamicum | 21851 | | | | | | | |
| Corynebacterium | glutamicum | 21253 | | | | | | | |
| Corynebacterium | glutamicum | 21514 | | | | | | | |
| Corynebacterium | glutamicum | 21516 | | | | | | | |
| Corynebacterium | glutamicum | 21299 | | | | | | | |
| Corynebacterium | glutamicum | 21300 | | | | | | | |
| Corynebacterium | glutamicum | 39684 | | | | | | | |
| Corynebacterium | glutamicum | 21488 | | | | | | | |
| Corynebacterium | glutamicum | 21649 | | | | | | | |
| Corynebacterium | glutamicum | 21650 | | | | | | | |
| Corynebacterium | glutamicum | 19223 | | | | | | | |
| Corynebacterium | glutamicum | 13869 | | | | | | | |
| Corynebacterium | glutamicum | 21157 | | | | | | | |
| Corynebacterium | glutamicum | 21158 | | | | | | | |
| Corynebacterium | glutamicum | 21159 | | | | | | | |
| Corynebacterium | glutamicum | 21355 | | | | | | | |
| Corynebacterium | glutamicum | 31808 | | | | | | | |
| Corynebacterium | glutamicum | 21674 | | | | | | | |
| Corynebacterium | glutamicum | 21562 | | | | | | | |
| Corynebacterium | glutamicum | 21563 | | | | | | | |
| Corynebacterium | glutamicum | 21564 | | | | | | | |
| Corynebacterium | glutamicum | 21565 | | | | | | | |
| Corynebacterium | glutamicum | 21566 | | | | | | | |
| Corynebacterium | glutamicum | 21567 | | | | | | | |
| Corynebacterium | glutamicum | 21568 | | | | | | | |
| Corynebacterium | glutamicum | 21569 | | | | | | | |
| Corynebacterium | glutamicum | 21570 | | | | | | | |
| Corynebacterium | glutamicum | 21571 | | | | | | | |
| Corynebacterium | glutamicum | 21572 | | | | | | | |
| Corynebacterium | glutamicum | 21573 | | | | | | | |
| Corynebacterium | glutamicum | 21579 | | | | | | | |
| Corynebacterium | glutamicum | 19049 | | | | | | | |
| Corynebacterium | glutamicum | 19050 | | | | | | | |
| Corynebacterium | glutamicum | 19051 | | | | | | | |
| Corynebacterium | glutamicum | 19052 | | | | | | | |
| Corynebacterium | glutamicum | 19053 | | | | | | | |
| Corynebacterium | glutamicum | 19054 | | | | | | | |
| Corynebacterium | glutamicum | 19055 | | | | | | | |
| Corynebacterium | glutamicum | 19056 | | | | | | | |
| Corynebacterium | glutamicum | 19057 | | | | | | | |
| Corynebacterium | glutamicum | 19058 | | | | | | | |
| Corynebacterium | glutamicum | 19059 | | | | | | | |
| Corynebacterium | glutamicum | 19060 | | | | | | | |
| Corynebacterium | glutamicum | 19185 | | | | | | | |
| Corynebacterium | glutamicum | 13286 | | | | | | | |
| Corynebacterium | glutamicum | 21515 | | | | | | | |
| Corynebacterium | glutamicum | 21527 | | | | | | | |
| Corynebacterium | glutamicum | 21544 | | | | | | | |
| Corynebacterium | glutamicum | 21492 | | | | | | | |
| Corynebacterium | glutamicum | | | B8183 | | | | | |
| Corynebacterium | glutamicum | | | B8182 | | | | | |
| Corynebacterium | glutamicum | | | B12416 | | | | | |
| Corynebacterium | glutamicum | | | B12417 | | | | | |
| Corynebacterium | glutamicum | | | B12418 | | | | | |
| Corynebacterium | glutamicum | | | B11476 | | | | | |
| Corynebacterium | glutamicum | 21608 | | | | | | | |
| Corynebacterium | lilium | | P973 | | | | | | |
| Corynebacterium | nitrilophilus | 21419 | | | | 11594 | | | |
| Corynebacterium | spec. | | P4445 | | | | | | |
| Corynebacterium | spec. | | P4446 | | | | | | |
| Corynebacterium | spec. | 31088 | | | | | | | |
| Corynebacterium | spec. | 31089 | | | | | | | |
| Corynebacterium | spec. | 31090 | | | | | | | |
| Corynebacterium | spec. | 31090 | | | | | | | |
| Corynebacterium | spec. | 31090 | | | | | | | |
| Corynebacterium | spec. | 15954 | | | | | | | 20145 |
| Corynebacterium | spec. | 21857 | | | | | | | |
| Corynebacterium | spec. | 21862 | | | | | | | |
| Corynebacterium | spec. | 21863 | | | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baarn, NL
NCTC: National Collection of Type Cultures, London, UK
DSMZ: Deutsche Sammlung von Mikroorganismen und Zellkulturen, Braunschweig, Germany
For reference see Sugawara, H. et al. (1993) World directory of collections of cultures of microorganisms: Bacteria, fungi and yeasts (4^{th} edn), World federation for culture collections world data center on microorganisms, Saimata, Japen.

TABLE 4
ALIGNMENT RESULTS

| | length | | | | | | % homology | Date of |
|---|---|---|---|---|---|---|---|---|
| ID # | (NT) | Genbank Hit | Length | Accession | Name of Genbank Hit | Source of Genbank Hit | (GAP) | Deposit |
| rxa00005 | 1731 | GB_BA1:CGGLNA | 3686 | Y13221 | Corynebacterium glutamicum glnA gene. | Corynebacterium glutamicum | 37,555 | 28-Aug-97 |
| | | GB_BA1:CGPROMF45 | 60 | X90363 | C. glutamicum DNA for promoter fragment F45. | Corynebacterium glutamicum | 100,000 | 4-Nov-96 |
| | | GB_BA1:CGGLNA | 3686 | Y13221 | Corynebacterium glutamicum glnA gene. | Corynebacterium glutamicum | 37,251 | 28-Aug-97 |
| rxa00011 | 480 | GB_BA1:D86821 | 5585 | D86821 | Streptomyces coelicolor DNA for PkaA, PkaB and PrfB, complete cds. | Streptomyces coelicolor | 69,729 | 7-Feb-99 |
| | | GB_BA1:MTCY164 | 39150 | Z95150 | Mycobacterium tuberculosis H37Rv complete genome; segment 135/162. | Mycobacterium tuberculosis | 35,639 | 19-Jun-98 |
| | | GB_BA1:MLCB1779 | 43254 | Z98271 | Mycobacterium leprae cosmid B1779. | Mycobacterium leprae | 37,555 | 8-Aug-97 |
| rxa00012 | 999 | GB_BA1:D86821 | 5585 | D86821 | Streptomyces coelicolor DNA for PkaA, PkaB and PrfB, complete cds. | Streptomyces coelicolor | 63,089 | 7-Feb-99 |
| | | GB_BA1:MTCY164 | 39150 | Z95150 | Mycobacterium tuberculosis H37Rv complete genome; segment 135/162. | Mycobacterium tuberculosis | 38,985 | 19-Jun-98 |
| | | GB_BA1:MLCB1779 | 43254 | Z98271 | Mycobacterium leprae cosmid B1779. | Mycobacterium leprae | 37,448 | 8-Aug-97 |
| rxa00016 | 1017 | GB_BA1:CGISABL | 1290 | X69104 | C. glutamicum IS3 related insertion element. | Corynebacterium glutamicum | 82,891 | 9-Aug-95 |
| | | GB_PAT:E12760 | 1279 | E12760 | DNA encoding Brevibacterium transposase. | Corynebacterium glutamicum | 83,201 | 24-Jun-98 |
| | | GB_PAT:AR038104 | 1279 | AR038104 | Sequence 9 from patent U.S. Pat. No. 5804414. | Unknown. | 83,201 | 29-Sep-99 |
| rxa00017 | 417 | GB_BA1:CGISABL | 1290 | X69104 | C. glutamicum IS3 related insertion element. | Corynebacterium glutamicum | 78,947 | 9-Aug-95 |
| | | GB_PAT:E12760 | 1279 | E12760 | DNA encoding Brevibacterium transposase. | Corynebacterium glutamicum | 77,895 | 24-Jun-98 |
| | | GB_PAT:AR038104 | 1279 | AR038104 | Sequence 9 from patent U.S. Pat. No. 5804414. | Unknown. | 77,895 | 29-Sep-99 |
| rxa00019 | 1983 | GB_PR3:HSN20A6 | 30875 | Z69713 | Human DNA sequence from cosmid cN20A6, on chromosome 22 contains STS, | Homo sapiens | 37,596 | 23-Nov-99 |
| | | GB_HTG3:AC011577 | 151996 | AC011577 | Homo sapiens clone 12_P_19, LOW-PASS SEQUENCE SAMPLING. | Homo sapiens | 34,506 | 07-OCT- |
| | | | | | | | | 1999 |
| | | GB_EST37:AW000587 | 470 | AW000587 | 614056A09.x1 614 - root cDNA library from Walbot Lab Zea mays | Zea mays | 41,578 | 8-Sep-99 |
| | | | | | cDNA, mRNA sequence. | | | |
| rxa00046 | 819 | GB_EST17:C73675 | 391 | C73675 | C73675 Rice panicle (longer than 10 cm) Oryza sativa cDNA clone E20126_2A, | Oryza sativa | 42,014 | 23-Sep-97 |
| | | | | | mRNA sequence. | | | |
| | | GB_EST31:AI704169 | 275 | AI704169 | UI-R-AC0-yi-d-08-0-UI.s1 UI-R-AC0 Rattus norvegicus cDNA clone | Rattus norvegicus | 38,182 | 3-Jun-99 |
| | | | | | UI-R-AC0-yi-d-08-0-UI 3′, mRNA sequence. | | | |
| | | GB_EST35:AI846250 | 390 | AI846250 | UI-M-AK1-aez-b-06-0-UI.s1 NIH_BMAP_MHY_N Mus musculus cDNA clone | Mus musculus | 34,872 | 15-Jul-99 |
| | | | | | UI-M-AK1-aez-b-06-0-UI 3′, mRNA sequence. | | | |
| rxa00053 | 516 | GB_PL2:AF072675 | 3127 | AF072675 | Kluyveromyces lactis Hap4p (HAP4) gene, complete cds. | Kluyveromyces lactis | 36,914 | 13-MAR- |
| | | | | | | | | 1999 |
| | | GB_VI:AB010886 | 3387 | AB010886 | Cydia pomonella granulovirus genes for chitinase and cathepsin, complete cds. | Cydia pomonella granulovirus | 35,375 | 13-Feb-99 |
| | | GB_PR2:HS1177I5 | 96256 | AL022315 | Human DNA sequence from clone 1177I5 on chromosome 22q13.1. Contains part | Homo sapiens | 36,884 | 23-Nov-99 |
| | | | | | of a putative novel gene, the gene for serum constituent protein MSE55 | | | |
| | | | | | downstream of a putative CpG island and the LGALS2 gene for Lectin, | | | |
| | | | | | Galactose-binding, soluble, 2 (Galectin 2, S-Lac Lectin 2, HL14). Contains | | | |
| | | | | | ESTs and GSSs, complete sequence. | | | |
| rxa00057 | 222 | GB_BA1:CGISABL | 1290 | X69104 | C. glutamicum IS3 related insertion element. | Corynebacterium glutamicum | 61,261 | 9-Aug-95 |
| | | GB_PAT:AR038104 | 1279 | AR038104 | Sequence 9 from patent U.S. Pat. No. 5804414. | Unknown. | 66,512 | 29-Sep-99 |
| | | GB_PAT:E12760 | 1279 | E12760 | DNA encoding Brevibacterium transposase. | Corynebacterium glutamicum | 66,512 | 24-Jun-98 |
| rxa00069 | 1506 | GB_BA1:MTCY27 | 27548 | Z95208 | Mycobacterium tuberculosis H37Rv complete genome; segment 104/162. | Mycobacterium tuberculosis | 38,029 | 17-Jun-98 |
| | | GB_BA1:MSGB1229CS | 30670 | L78812 | Mycobacterium leprae cosmid B1229 DNA sequence. | Mycobacterium leprae | 64,940 | 15-Jun-96 |
| | | GB_BA1:MSGB998CS | 10000 | L78829 | Mycobacterium leprae cosmid B998 DNA sequence. | Mycobacterium leprae | 64,940 | 15-Jun-96 |
| rxa00102 | 891 | GB_PR3:HS45P21 | 170001 | AL021917 | Human DNA sequence from clone 45P21 on chromosome 6p21.3-22.2 Contains | Homo sapiens | 37,882 | 23-Nov-99 |
| | | | | | butyrophilins (BTF3, BTF5, BTF2, BTF4), EST, STS, complete sequence. | | | |
| | | GB_PR3:AC005330 | 40607 | AC005330 | Homo sapiens chromosome 19, cosmid R34047, complete sequence. | Homo sapiens | 35,666 | 28-Jul-98 |
| | | GB_GSS4:AQ677431 | 502 | AQ677431 | HS_5529_A2_C01_T7A RPCI-11 Human Male BAC Library Homo sapiens | Homo sapiens | 37,000 | 25-Jun-99 |
| | | | | | genomic clone Plate = 1105 Col = 2 Row = E, genomic survey sequence. | | | |
| rxa00107 | 360 | GB_PL2:AC007153 | 103223 | AC007153 | Arabidopsis thaliana chromosome I BAC F3F20 genomic sequence, complete | Arabidopsis thaliana | 33,427 | 17-MAY- |
| | | | | | sequence. | | | 1999 |
| | | GB_BA2:PPU89363 | 4642 | U89363 | Pseudomonas putida P38K, amidase, nitrile hydratase alpha subunit, nitrile | Pseudomonas putida | 40,988 | 2-Jun-98 |
| | | | | | hydratase beta subunit, and P14K genes, complete cds. | | | |
| | | GB_PAT:AR041193 | 1440 | AR041193 | Sequence 17 from patent U.S. Pat. No. 5811286. | Unknown. | 40,988 | 29-Sep-99 |
| rxa00125 | 888 | EM_PAT:E09053 | 2538 | E09053 | gDNA encoding secA protein. | Corynebacterium glutamicum | 94,028 | 07-OCT- |
| | | | | | | | | 1997 (Rel. |
| | | | | | | | | 52, Created) |
| | | GB_BA1:MSU66081 | 2968 | U66081 | Mycobacterium smegmatis SecA (SecA) gene, complete cds. | Mycobacterium smegmatis | 71,216 | 28-Aug-98 |
| | | GB_BA2:SLU21192 | 4006 | U21192 | Streptomyces lividans SecA (secA) gene, complete cds. | Streptomyces lividans | 63,472 | 3-Sep-96 |
| rxa00138 | 684 | GB_BA1:BLELONP | 738 | X99289 | B. lactofermentum gene encoding elongation factor P. | Corynebacterium glutamicum | 98,331 | 1-Nov-97 |
| | | GB_BA1:MTCY159 | 33818 | Z83863 | Mycobacterium tuberculosis H37Rv complete genome; segment 111/162. | Mycobacterium tuberculosis | 37,946 | 17-Jun-98 |
| | | GB_BA1:MSGB937CS | 38914 | L78820 | Mycobacterium leprae cosmid B937 DNA sequence. | Mycobacterium leprae | 62,261 | 15-Jun-96 |
| rxa00172 | 735 | GB_EST28:AI484755 | 572 | AI484755 | EST243016 tomato ovary, TAMU Lycopersicon esculentum cDNA clone | Lycopersicon esculentum | 39,171 | 29-Jun-99 |
| | | | | | cLED3O13, mRNA sequence. | | | |
| | | GB_EST28:AI486041 | 610 | AI486041 | EST244362 tomato ovary, TAMU Lycopersicon esculentum | Lycopersicon esculentum | 46,452 | 29-Jun-99 |
| | | | | | cDNA clone cLED2K7, mRNA sequence. | | | |
| | | GB_PR3:HS396D17 | 152592 | AL008634 | Human DNA sequence from clone 396D17 on chromosome 1p33-35.3 Contains | Homo sapiens | 33,060 | 23-Nov-99 |
| | | | | | EST, STS, GSS, complete sequence. | | | |
| rxa00184 | 1296 | GB_BA1:MTCY50 | 36030 | Z77137 | Mycobacterium tuberculosis H37Rv complete genome; segment 55/162. | Mycobacterium tuberculosis | 47,823 | 17-Jun-98 |
| | | GB_BA1:AB013492 | 18497 | AB013492 | Bacillus halodurans C-125 genomic DNA, 9A/3S′ fragment, clone ALBAC001. | Bacillus halodurans | 39,234 | 3-Aug-99 |
| | | GB_PR3:AC005738 | 134506 | AC005738 | Homo sapiens chromosome 5, BAC clone 7g12 | Homo sapiens | 37,127 | 20-OCT- |
| | | | | | (LBNL H126), complete sequence. | | | 1998 |
| rxa00209 | 1614 | GB_BA1:MTV012 | 70287 | AL021287 | Mycobacterium tuberculosis H37Rv complete genome; segment 132/162. | Mycobacterium tuberculosis | 37,632 | 23-Jun-99 |
| | | GB_BA1:MLCB637 | 44882 | Z99263 | Mycobacterium leprae cosmid B637. | Mycobacterium leprae | 65,785 | 17-Sep-97 |
| | | GB_BA1:SC8D9 | 38681 | AL035569 | Streptomyces coelicolor cosmid 8D9. | Streptomyces coelicolor | 63,795 | 26-Feb-99 |
| rxa00210 | 420 | GB_PL1:MGR7031 | 103 | AJ007031 | Mycosphaerella graminicola microsatellite ST1A2 DNA. | Mycosphaerella graminicola | 45,545 | 3-Aug-98 |
| | | GB_HTG1:CEY48G10_4 | 110000 | AL021450 | Caenorhabditis elegans chromosome 1 clone Y48G10, *** SEQUENCING IN | Caenorhabditis elegans | 37,101 | 29-Jul-99 |
| | | | | | PROGRESS ***, in unordered pieces. | | | |
| | | GB_HTG1:CEY48G10_4 | 110000 | AL021450 | Caenorhabditis elegans chromosome 1 clone Y48G10, *** SEQUENCING IN | Caenorhabditis elegans | 37,101 | 29-Jul-99 |
| | | | | | PROGRESS ***, in unordered pieces. | | | |
| rxa00217 | 1218 | GB_BA1:MTV012 | 70287 | AL021287 | Mycobacterium tuberculosis H37Rv complete genome; segment 132/162. | Mycobacterium tuberculosis | 35,122 | 23-Jun-99 |
| | | GB_HTG2:AC008092 | 88749 | AC008092 | Drosophila melanogaster chromosome 3 clone BACR22F22 (D824) RPCI-98 | Drosophila melanogaster | 33,001 | 2-Aug-99 |
| | | | | | 22.F.22 map 84D-84D strain y; cn bw sp, *** SEQUENCING | | | |
| | | | | | IN PROGRESS***, 53 unordered pieces. | | | |
| | | GB_HTG2:AC008092 | 88749 | AC008092 | Drosophila melanogaster chromosome 3 clone BACR22F22 (D824) RPCI-98 | Drosophila melanogaster | 33,001 | 2-Aug-99 |
| | | | | | 22.F.22 map 84D-84D strain y; cn bw sp, *** SEQUENCING | | | |
| | | | | | IN PROGRESS***, 53 unordered pieces. | | | |
| rxa00227 | 921 | GB_BA1:LPLLDHE | 1651 | X70926 | L. plantarum gene for 1-lactate dehydrogenase. | Lactobacillus plantarum | 37,294 | 17-Feb-94 |
| | | GB_GSS9:AQ158656 | 731 | AQ158656 | nbxb0011N08f CUGI Rice BAC Library Oryza sativa | Oryza sativa | 39,041 | 12-Sep-98 |
| | | | | | genomic clone nbxb0011N08f, genomic survey sequence. | | | |
| | | GB_BA1:LPLLDHE | 1651 | X70926 | L. plantarum gene for 1-lactate dehydrogenase. | Lactobacillus plantarum | 34,947 | 17-Feb-94 |
| rxa00265 | 573 | GB_PR4:AC007368 | 94024 | AC007368 | Homo sapiens 12q24.2 PAC RPCI4-809F18 (Roswell Park Cancer Institute | Homo sapiens | 40,037 | 31-Jul-99 |
| | | | | | Human PAC Library) complete sequence. | | | |
| | | GB_PR4:AC007368 | 94024 | AC007368 | Homo sapiens 12q24.2 PAC RPCI4-809F18 (Roswell Park Cancer Institute | Homo sapiens | 36,121 | 31-Jul-99 |
| | | | | | Human PAC Library) complete sequence. | | | |
| rxa00280 | 624 | GB_EST29:AI588595 | 532 | AI588595 | fb97b04.y1 Zebrafish WashU MPIMG EST Danio rerio cDNA 5′ similar to | Danio rerio | 34,242 | 21-Apr-99 |
| | | | | | WP: F32D8.4 CE05783 LACTATE DEHYDROGENASE;, mRNA sequence. | | | |
| | | GB_VI:D78362 | 1593 | D78362 | Rotavirus sp. mRNA for nonstructural protein 1, complete cds. | Rotavirus sp. | 35,217 | 22-Jan-98 |
| | | GB_EST29:AI588595 | 532 | AI588595 | fb97b04.y1 Zebrafish WashU MPIMG EST Danio rerio cDNA 5′ similar to | Danio rerio | 36,118 | 21-Apr-99 |
| | | | | | WP: F32D8.4 CE05783 LACTATE DEHYDROGENASE;, mRNA sequence. | | | |
| rxa00314 | 1503 | GB_PAT:AR008345 | 1344 | AR008345 | Sequence 1 from patent U.S. Pat. No. 5753480. | Unknown. | 50,783 | 04-DEC- |
| | | | | | | | | 1998 |
| | | GB_BA1:ABIPDC | 4933 | X99587 | A. brasilense ipdC, gltX & cysS genes. | Azospirillum brasilense | 37,244 | 9-Jan-98 |
| | | GB_PAT:AR008346 | 333 | AR008346 | Sequence 3 from patent U.S. Pat. No. 5753480. | Unknown. | 64,545 | 04-DEC- |
| | | | | | | | | 1998 |
| rxa00331 | 480 | GB_BA1:CGTHRC | 3120 | X56037 | Corynebacterium glutamicum thrC gene for threonine synthase (EC4.2.99.2). | Corynebacterium glutamicum | 40,393 | 17-Jun-97 |
| | | GB_PAT:I09078 | 3146 | I09078 | Sequence 4 from Patent WO 8809819. | Unknown. | 38,462 | 02-DEC- |
| | | | | | | | | 1994 |
| | | GB_BA1:SAY14370 | 7791 | Y14370 | Staphylococcus aureus RF3, murE, ypfP genes. | Staphylococcus aureus | 34,526 | 24-Jun-98 |
| rxa00333 | 657 | GB_PR3:AC004788 | 39436 | AC004788 | Homo sapiens chromosome 7 clone UWGC: g1564a327 from 7p14-15, complete | Homo sapiens | 37,618 | 2-Jun-98 |
| | | | | | sequence. | | | |
| | | GB_PR3:AC004788 | 39436 | AC004788 | Homo sapiens chromosome 7 clone UWGC: g1564a327 from 7p14-15, complete | Homo sapiens | 34,169 | 2-Jun-98 |
| | | | | | sequence. | | | |
| rxa00454 | 1416 | GB_BA2:AE000147 | 10577 | AE000147 | Escherichia coli K-12 MG1655 section 37 of 400 of the complete genome. | Escherichia coli | 48,925 | 12-Nov-98 |
| | | GB_PR4:DJ270M14 | 192126 | AF107885 | Homo sapiens chromosome 14q24.3 clone BAC270M14 | Homo sapiens | 36,043 | 14-Jul-99 |
| | | | | | transforming growth factor beta 3 (TGF-beta 3) gene, | | | |
| | | | | | complete cds; and unknown genes. | | | |
| | | GB_BA1:ECOTGT | 1823 | M63939 | E. coli tRNA-guanine-transglycosylase (tgt) gene, complete cds. | Escherichia coli | 48,925 | 26-APR-199 |
| rxa00458 | 736 | GB_BA1:SC4G2 | 30590 | AL031371 | Streptomyces coelicolor cosmid 4G2. | Streptomyces coelicolor | 34,836 | 5-Sep-98 |
| | | GB_BA2:AF024619 | 4038 | AF024619 | Pseudomonas fluorescens hybrid histidine kinase homolog (styS) and response | Pseudomonas fluorescens | 39,251 | 23-MAR- |
| | | | | | regulatory protein (styR) genes, complete cds. | | | 1998 |
| | | GB_BA1:SC4G2 | 30590 | AL031371 | Streptomyces coelicolor cosmid 4G2. | Streptomyces coelicolor | 40,196 | 5-Sep-98 |
| rxa00484 | 1203 | GB_PL1:AB012627 | 3019 | AB012627 | Adiantum capillus-veneris CRY2 mRNA for blue-light | Adiantum capillus-veneris | 43,959 | 5-Feb-99 |
| | | | | | photoreceptor, complete cds. | | | |
| | | GB_PL1:AB012630 | 4098 | AB012630 | Adiantum capillus-veneris CRY2 gene for blue-light photoreceptor, complete cds. | Adiantum capillus-veneris | 39,765 | 5-Feb-99 |
| | | GB_PL1:YSCF6552A | 20383 | D31600 | Saccharomyces cerevisiae chromosome VI phage 6552. | Saccharomyces cerevisiae | 37,133 | 7-Feb-99 |
| rxa00495 | 687 | GB_HTG3:AC008853 | 54169 | AC008853 | Homo sapiens chromosome 5 clone CITB-H1_2176P21, *** SEQUENCING IN | Homo sapiens | 36,471 | 3-Aug-99 |
| | | | | | PROGRESS ***, 66 unordered pieces. | | | |
| | | GB_HTG3:AC008853 | 54169 | AC008853 | Homo sapiens chromosome 5 clone CITB-H1_2176P21, *** SEQUENCING IN | Homo sapiens | 36,471 | 3-Aug-99 |
| | | | | | PROGRESS ***, 66 unordered pieces. | | | |
| | | GB_HTG3:AC008853 | 54169 | AC008853 | Homo sapiens chromosome 5 clone CITB-H1_2176P21, *** SEQUENCING IN | Homo sapiens | 36,090 | 3-Aug-99 |
| | | | | | PROGRESS ***, 66 unordered pieces. | | | |
| rxa00532 | 608 | GB_BA1:ECR751 | 5499 | X54458 | E. coli plasmid R751 traF (5′end), traG, traH, traI, traJ, traK and | Escherichia coli | 38,992 | 18-Nov-93 |
| | | | | | traL (5′end) genes of the transfer region. | | | |
| | | GB_BA2:EAU67194 | 53339 | U67194 | Enterobacter aerogenes plasmid R751, complete plasmid sequence. | Enterobacter aerogenes | 38,992 | 19-OCT- |
| | | | | | | | | 1998 |
| | | GB_BA1:D83237 | 1626 | D83237 | Rhodococcus erythropolis DNA for catechol 1,2-dioxgenase, complete cds. | Rhodococcus erythropolis | 37,232 | 1-Sep-99 |
| rxa00539 | 600 | GB_PL2:AF053311 | 1110 | AF053311 | Zantedeschia aethiopica glutathione peroxidase (gpx) mRNA, nuclear gene | Zantedeschia aethiopica | 48,552 | 20-Nov-98 |
| | | | | | encoding chloroplast protein, complete cds. | | | |
| | | GB_PL2:AF053311 | 1110 | AF053311 | Zantedeschia aethiopica glutathione peroxidase (gpx) mRNA, nuclear gene | Zantedeschia aethiopica | 36,301 | 20-Nov-98 |
| | | | | | encoding chloroplast protein, complete cds. | | | |
| rxa00568 | 1470 | GB_PAT:I92046 | 2203 | I92046 | Sequence 13 from patent U.S. Pat. No. 5726299. | Unknown. | 37,129 | 01-DEC- |
| | | | | | | | | 1998 |
| | | GB_PAT:I78757 | 2203 | I78757 | Sequence 13 from patent U.S. Pat. No. 5693781. | Unknown. | 37,129 | 3-Apr-98 |
| | | GB_PR4:AC005042 | 192218 | AC005042 | Homo sapiens clone NH0552E01, complete sequence. | Homo sapiens | 37,672 | 14-Jan-99 |
| rxa00588 | 645 | GB_BA1:MTV017 | 67200 | AL021897 | Mycobacterium tuberculosis H37Rv complete genome; segment 48/162. | Mycobacterium tuberculosis | 36,150 | 24-Jun-99 |
| | | GB_BA1:PAU81259 | 7285 | U81259 | Pseudomonas aeruginosa dihydrodipicolinate reductase (dapB) gene, partial cds, | Pseudomonas aeruginosa | 45,483 | 23-DEC- |
| | | | | | carbamoylphosphate synthetase small subunit (carA) and carbamoylphosphate | | | 1996 |
| | | | | | synthetase large subunit (carB) genes, complete cds, and FtsJ homolog (ftsJ) | | | |
| | | | | | gene, partial cds. | | | |
| | | GB_IN2:AC005643 | 80389 | AC005643 | Drosophila melanogaster, chromosome 2R, region 50C5-50C8, | Drosophila melanogaster | 40,705 | 15-DEC- |
| | | | | | P1 clone DS02972, complete sequence. | | | 1998 |
| rxa00677 | 339 | GB_BA1:MLCB1222 | 34714 | AL049491 | Mycobacterium leprae cosmid B1222. | Mycobacterium leprae | 40,549 | 27-Aug-99 |
| | | GB_BA1:MBU15140 | 2136 | U15140 | Mycobacterium bovis ribosomal proteins IF-1 (infA), L36 (rpmJ), S13 (rpsM) and | Mycobacterium bovis | 64,881 | 28-OCT- |
| | | | | | S11 (rpsK) genes, complete cds, and S4 (rpsD) gene, partial cds. | | | 1996 |
| | | GB_BA1:MTY13E12 | 43401 | Z95390 | Mycobacterium tuberculosis H37Rv complete genome; segment 147/162. | Mycobacterium tuberculosis | 41,896 | 17-Jun-98 |
| rxa00687 | 1443 | GB_BA1:BRLSECY | 1516 | D14162 | Brevibacterium flavum gene for SecY protein (complete cds) and gene for | Corynebacterium glutamicum | 98,436 | 3-Feb-99 |
| | | | | | adenylate kinase (partial cds). | | | |
| | | GB_PAT:E07701 | 1323 | E07701 | Brevibacterium secY gene. | Corynebacterium glutamicum | 98,262 | 29-Sep-97 |
| | | GB_BA1:MTV041 | 28826 | AL021958 | Mycobacterium tuberculosis H37Rv complete genome; segment 35/162. | Mycobacterium tuberculosis | 60,724 | 17-Jun-98 |
| rxa00753 | 1704 | GB_EST17:C61980 | 216 | C61980 | C61980 Yuji Kohara unpublished cDNA Caenorhabditis elegans cDNA clone | Caenorhabditis elegans | 43,030 | 22-Sep-97 |
| | | | | | yk272b4 5′, mRNA sequence. | | | |
| | | GB_RO:MMANT12 | 5141 | X01815 | Mouse gene for H-2K(d) antigen. | Mus musculus | 37,317 | 03-OCT- |
| | | | | | | | | 1997 |
| | | GB_PR4:AC003001 | 101981 | AC003001 | Homo sapiens chromosome X, clone HRPC928E24, complete sequence. | Homo sapiens | 34,127 | 6-Feb-99 |
| rxa00824 | 681 | GB_PL2:ATFCA0 | 200576 | Z97335 | Arabidopsis thaliana DNA chromosome 4, ESSA I FCA contig fragment No. 0. | Arabidopsis thaliana | 36,527 | 28-Jun-99 |
| | | GB_PR4:AC006443 | 210636 | AC006443 | Homo sapiens chromosome 9, clone hRPK.494_N_15, complete sequence. | Homo sapiens | 38,401 | 30-Jan-99 |
| | | GB_PR4:AC006443 | 210636 | AC006443 | Homo sapiens chromosome 9, clone hRPK.494_N_15, complete sequence. | Homo sapiens | 34,027 | 30-Jan-99 |
| rxa00896 | 702 | GB_GSS12:AQ403148 | 432 | AQ403148 | HS_5052_A2_F07_SP6E RPCI-11 Human Male BAC Library Homo sapiens | Homo sapiens | 41,371 | 13-MAR- |
| | | | | | genomic clone Plate = 628 Col = 14 Row = K, genomic survey sequence. | | | 1999 |
| | | GB_HTG6:AC009921 | 184689 | AC009921 | Homo sapiens clone RP11-115O18, WORKING DRAFT SEQUENCE, 17 | Homo sapiens | 37,223 | 03-DEC- |
| | | | | | unordered pieces. | | | 1999 |
| | | GB_HTG6:AC009921 | 184689 | AC009921 | Homo sapiens clone RP11-115O18, WORKING DRAFT SEQUENCE, 17 | Homo sapiens | 38,438 | 03-DEC- |
| | | | | | unordered pieces. | | | 1999 |
| rxa00927 | 1212 | GB_BA1:MTCY227 | 35946 | Z77724 | Mycobacterium tuberculosis H37Rv complete genome; segment 114/162. | Mycobacterium tuberculosis | 36,493 | 17-Jun-98 |
| | | GB_BA1:U00011 | 40429 | U00011 | Mycobacterium leprae cosmid B1177. | Mycobacterium leprae | 37,978 | 01-MAR- |
| | | | | | | | | 1994 |
| | | GB_BA1:D90829 | 20277 | D90829 | E. coli genomic DNA, Kohara clone #337(41.9-42.3 min.). | Escherichia coli | 36,750 | 21-MAR- |
| | | | | | | | | 1997 |
| rxa00928 | 741 | GB_PR2:HS1121J18 | 138145 | AL031653 | Human DNA sequence from clone 1121J18 on chromosome 20. Contains ESTs, | Homo sapiens | 37,997 | 23-Nov-99 |
| | | | | | STS, GSSs, a ca repeat polymorphism and genomic marker D20S115′, complete | | | |
| | | | | | sequence. | | | |
| | | GB_PR2:HS1121J18 | 138145 | AL031653 | Human DNA sequence from clone 1121J18 on chromosome 20. Contains ESTs, | Homo sapiens | 38,701 | 23-Nov-99 |
| | | | | | STS, GSSs, a ca repeat polymorphism and genomic marker D20S115′, complete | | | |
| | | | | | sequence. | | | |
| | | GB_HTG3:AC008715 | 101012 | AC008715 | Homo sapiens chromosome 5 clone CIT978SKB_84H3, *** SEQUENCING IN | Homo sapiens | 38,199 | 3-Aug-99 |
| | | | | | PROGRESS ***, 24 unordered pieces. | | | |
| rxa00929 | 786 | GB_HTG3:AC004480 | 220000 | AC004480 | Homo sapiens chromosome 4, *** SEQUENCING IN PROGRESS ***, | Homo sapiens | 37,131 | 2-Sep-99 |
| | | | | | 7 unordered pieces. | | | |
| | | GB_HTG3:AC004480 | 220000 | AC004480 | Homo sapiens chromosome 4, *** SEQUENCING IN PROGRESS ***, | Homo sapiens | 37,131 | 2-Sep-99 |
| | | | | | 7 unordered pieces. | | | |
| | | GB_HTG3:AC004480 | 220000 | AC004480 | Homo sapiens chromosome 4, *** SEQUENCING IN PROGRESS ***, | Homo sapiens | 37,775 | 2-Sep-99 |
| | | | | | 7 unordered pieces. | | | |
| rxa00937 | 495 | GB_GSS3:B67258 | 592 | B67258 | T23N5TF TAMU Arabidopsis thaliana genomic clone T23N5, genomic survey | Arabidopsis thaliana | 35,644 | 09-DEC- |
| | | | | | sequence. | | | 1997 |
| | | GB_PL2:ATAC006413 | 96059 | AC006413 | Arabidopsis thaliana chromosome II BAC F5K7 genomic sequence, complete | Arabidopsis thaliana | 36,864 | 09-MAR- |
| | | | | | sequence. | | | 1999 |
| | | GB_EST8:AA052151 | 282 | AA052151 | mf81g03.r1 Soares mouse embryo NbME13.5 14.5 Mus musculus cDNA clone | Mus musculus | 38,652 | 13-Sep-96 |
| | | | | | IMAGE: 420724 5′, mRNA sequence. | | | |
| rxa00938 | 381 | GB_BA2:AF121000 | 19751 | AF121000 | Corynebacterium glutamicum strain 22243 R-plasmid pAG1, complete sequence. | Corynebacterium glutamicum | 39,410 | 14-Apr-99 |
| | | GB_BA1:FVBPOAD2A | 45519 | D26094 | Flavobacterium sp. plasmid pOAD2 DNA, whole sequence. | Flavobacterium sp. | 37,228 | 6-Feb-99 |
| | | GB_BA1:FVBPOAD2A | 45519 | D26094 | Flavobacterium sp. plasmid pOAD2 DNA, whole sequence. | Flavobacterium sp. | 63,102 | 6-Feb-99 |
| rxa00966 | 640 | GB_BA1:MLCB628 | 40789 | Y14967 | Mycobacterium leprae cosmid B628. | Mycobacterium leprae | 60,938 | 29-Aug-97 |
| | | GB_BA1:MLCB1770 | 37821 | Z70722 | Mycobacterium leprae cosmid B1770. | Mycobacterium leprae | 60,938 | 29-Aug-97 |
| | | GB_BA1:MTCY21D4 | 20760 | Z80775 | Mycobacterium tuberculosis H37Rv complete genome; segment 3/262. | Mycobacterium tuberculosis | 59,375 | 24-Jun-99 |
| rxa00968 | 1054 | GB_BA1:MSGY219 | 38721 | AD000013 | Mycobacterium tuberculosis sequence from clone y219. | Mycobacterium tuberculosis | 36,077 | 10-DEC- |
| | | | | | | | | 1996 |
| | | GB_BA1:MTCY21D4 | 20760 | Z80775 | Mycobacterium tuberculosis H37Rv complete genome; segment 3/262. | Mycobacterium tuberculosis | 67,536 | 24-Jun-99 |
| | | GB_BA1:MLCB628 | 40789 | Y14967 | Mycobacterium leprae cosmid B628. | Mycobacterium leprae | 65,990 | 29-Aug-97 |
| rxa00975 | 1773 | GB_PAT:E14508 | 3579 | E14508 | DNA encoding Brevibacterium diaminopimelic acid decarboxylase and arginyl- | Corynebacterium glutamicum | 99,887 | 28-Jul-99 |
| | | | | | tRNA synthase. | | | |
| | | GB_PAT:AR038110 | 3579 | AR038110 | Sequence 15 from patent U.S. Pat. No. 5804414. | Unknown. | 99,887 | 29-Sep-99 |
| | | GB_PAT:E16355 | 3579 | E16355 | Brevibacterium argS and lysA genes. | Corynebacterium glutamicum | 99,887 | 28-Jul-99 |
| rxa00978 | 738 | GB_PR2:HSAC000372 | 41730 | AC000372 | Human cosmid g1980a186, complete sequence. | Homo sapiens | 34,674 | 12-MAR- |
| | | | | | | | | 1997 |
| | | GB_PR3:AC005503 | 40998 | AC005503 | Homo sapiens clone UWGC:g5129s003 from 7q31, complete sequence. | Homo sapiens | 34,674 | 20-Aug-98 |
| | | GB_PR2:HSAC000372 | 41730 | AC000372 | Human cosmid g1980a186, complete sequence. | Homo sapiens | 38,881 | 12-MAR- |
| | | | | | | | | 1997 |
| rxa00985 | 2832 | GB_BA1:MTV008 | 63033 | AL021246 | Mycobacterium tuberculosis H37Rv complete genome; segment 108/162. | Mycobacterium tuberculosis | 38,126 | 17-Jun-98 |
| | | GB_BA1:BSVALTRS | 3168 | X77239 | B. subtilis valS gene. | Bacillus subtilis | 52,036 | 16-Apr-97 |
| | | GB_BA1:ECOUW93 | 338534 | U14003 | Escherichia coli K-12 chromosomal region from 92.8 to 00.1 minutes. | Escherichia coli | 37,971 | 17-Apr-96 |
| rxa00998 | 585 | GB_PAT:E13660 | 1916 | E13660 | gDNA encoding 6-phosphogluconate dehydrogenase. | Corynebacterium glutamicum | 38,398 | 24-Jun-98 |
| | | GB_HTG2:AF164115 | 94757 | AF164115 | Homo sapiens chromosome 8 clone BAC 644F11, *** SEQUENCING IN | Homo sapiens | 33,563 | 12-Jul-99 |
| | | | | | PROGRESS ***, in unordered pieces. | | | |
| | | GB_HTG2:AF164115 | 94757 | AF164115 | Homo sapiens chromosome 8 clone BAC 644F11, *** SEQUENCING IN | Homo sapiens | 33,563 | 12-Jul-99 |
| | | | | | PROGRESS ***, in unordered pieces. | | | |
| rxa01020 | 870 | GB_EST29:AI553731 | 416 | AI553731 | tn28b06.x1 NCI_CGAP_Brn25 Homo sapiens cDNA clone IMAGE: 2168915 3′ | Homo sapiens | 36,855 | 12-MAY- |
| | | | | | similar to contains element TAR1 TAR1 repetitive element;, mRNA sequence. | | | 1999 |
| | | GB_EST35:AI871115 | 506 | AI871115 | wI79c08.x1 NCI_CGAP_Brn25 Homo sapiens cDNA clone IMAGE: 2431118 3′ | Homo sapiens | 37,549 | 30-Aug-99 |
| | | | | | similar to TR:O75176 O75176 KIAA0692 PROTEIN; contains element MER15 | | | |
| | | | | | repetitive element;, mRNA sequence. | | | |
| | | GB_EST27:AI430328 | 520 | AI430328 | mf66a05.y1 Soares mouse embryo NbME13.5 14.5 Mus musculus cDNA clone | Mus musculus | 37,765 | 09-MAR- |
| | | | | | IMAGE: 419216 5′, mRNA sequence. | | | 1999 |
| rxa01061 | 1061 | GB_BA1:MTCY21D4 | 20760 | Z80775 | Mycobacterium tuberculosis H37Rv complete genome; segment 3/262. | Mycobacterium tuberculosis | 62,606 | 24-Jun-99 |
| | | GB_BA1:MSGY219 | 38721 | AD000013 | Mycobacterium tuberculosis sequence from clone y219. | Mycobacterium tuberculosis | 41,171 | 10-DEC- |
| | | | | | | | | 1996 |
| | | GB_BA1:MLCB628 | 40789 | Y14967 | Mycobacterium leprae cosmid B628. | Mycobacterium leprae | 61,022 | 29-Aug-97 |
| rxa01072 | 354 | GB_BA2:AF112535 | 4363 | AF112535 | Corynebacterium glutamicum putative glutaredoxin NrdH (nrdH), NrdI (nrdI), | Corynebacterium glutamicum | 99,718 | 5-Aug-99 |
| | | | | | and ribonucleotide reductase alpha-chain (nrdE) genes, complete cds. | | | |
| | | GB_BA1:CANRDFGEN | 6054 | Y09572 | Corynebacterium ammoniagenes nrdH, nrdI, nrdE, nrdF genes. | Corynebacterium | 62,393 | 18-Apr-98 |
| | | | | | | ammoniagenes | | |
| | | GB_BA1:MTCY22D7 | 31859 | Z83866 | Mycobacterium tuberculosis H37Rv complete genome; segment 133/162. | Mycobacterium tuberculosis | 37,714 | 17-Jun-98 |
| rxa01124 | 1602 | GB_BA1:SC1C2 | 42210 | AL031124 | Streptomyces coelicolor cosmid 1C2. | Streptomyces coelicolor | 60,616 | 15-Jan-99 |
| | | GB_BA1:MTV012 | 70287 | AL021287 | Mycobacterium tuberculosis H37Rv complete genome; segment 132/162. | Mycobacterium tuberculosis | 37,913 | 23-Jun-99 |
| | | GB_BA1:MLCB637 | 44882 | Z99263 | Mycobacterium leprae cosmid B637. | Mycobacterium leprae | 61,216 | 17-Sep-97 |
| rxa01199 | 871 | GB_PR3:AF046873 | 2153 | AF046873 | Homo sapiens synapsin IIIa mRNA, complete cds. | Homo sapiens | 37,184 | 28-Apr-98 |
| | | GB_EST30:AI649049 | 691 | AI649049 | uk34f03.x1 Sugano mouse kidney mkia Mus musculus cDNA clone | Mus musculus | 37,226 | 30-Apr-99 |
| | | | | | IMAGE: 1970909 3′ similar to gb: X15684 Mouse mRNA for liver-type glucose | | | |
| | | | | | transporter protein (MOUSE);, mRNA sequence. | | | |
| | | GB_EST23:AI121163 | 468 | AI121163 | ud70b04.x1 Sugano mouse liver mlia Mus musculus cDNA clone IMAGE: | Mus musculus | 35,057 | 2-Sep-98 |
| | | | | | 1451215 3′ similar to gb: J03810 GLUCOSE TRANSPORTER TYPE 2, | | | |
| | | | | | LIVER (HUMAN); gb: X15684 Mouse mRNA for liver-type glucose | | | |
| | | | | | transporter protein (MOUSE);, mRNA sequence. | | | |
| rxa01223 | 735 | GB_PR4:AC007386 | 176742 | AC007386 | Homo sapiens BAC clone NH0359K10 from 2, complete sequence. | Homo sapiens | 39,551 | 22-OCT- |
| | | | | | | | | 1999 |
| | | GB_PR4:AC007386 | 176742 | AC007386 | Homo sapiens BAC clone NH0359K10 from 2, complete sequence. | Homo sapiens | 38,678 | 22-OCT- |
| | | | | | | | | 1999 |
| rxa01226 | 663 | GB_PR2:HS21F7 | 150789 | AL033375 | Human DNA sequence from clone 21F7 on chromosome 6q16.1-21. Contains part | Homo sapiens | 37,309 | 23-Nov-99 |
| | | | | | of an exon of a putative new gene and STSs and GSSs, complete sequence. | | | |
| | | GB_PR3:AF023268 | 75270 | AF023268 | Homo sapiens clk2 kinase (CLK2), propin1, cote1, glucocerebrosidase (GBA), | Homo sapiens | 38,923 | 28-OCT- |
| | | | | | and metaxin genes, complete cds; metaxin pseudogene and glucocerebrosidase | | | 1997 |
| | | | | | pseudogene; and thrombospondin3 (THBS3) gene, partial cds. | | | |
| | | GB_BA2:AF016485 | 191346 | AF016485 | Halobacterium sp. NRC-1 plasmid pNRC100, complete plasmid sequence. | Halobacterium sp. NRC-1 | 39,938 | 29-MAR- |
| | | | | | | | | 1999 |
| rxa01228 | 339 | GB_PR2:HS1158E12 | 163871 | AL031584 | Human DNA sequence from clone 1158E12 on chromosome Xp11.23-11.4 | Homo sapiens | 34,718 | 23-Nov-99 |
| | | | | | Contains EST, STS, GSS, CpG island, complete sequence. | | | |
| | | GB_HTG6:AC008180_0 | 110000 | AC008180 | Homo sapiens clone RP11-292L5, *** SEQUENCING IN PROGRESS ***, 152 | Homo sapiens | 31,212 | 29-Jul-99 |
| | | | | | unordered pieces. | | | |
| | | GB_PR4:AC004908 | 138251 | AC004908 | Homo sapiens PAC clone DJ0855D21, complete sequence. | Homo sapiens | 37,082 | 15-Jan-99 |
| rxa01252 | 777 | GB_BA1:MTV025 | 121125 | AL022121 | Mycobacterium tuberculosis H37Rv complete genome; segment 155/162. | Mycobacterium tuberculosis | 39,171 | 24-Jun-99 |
| | | GB_BA1:SC66T3 | 35101 | AL079348 | Streptomyces coelicolor cosmid 66T3. | Streptomyces coelicolor | 35,401 | 19-Jun-99 |
| | | GB_BA2:AF151381 | 1296 | AF151381 | Streptomyces coelicolor recombination protein RecR (recR) gene, complete cds; | Streptomyces coelicolor | 53,826 | 20-Aug-99 |
| | | | | | and unknown gene. | | | |
| rxa01264 | 339 | GB_GSS10:AQ195163 | 617 | AQ195163 | RPCI11-66I23.TJ RPCI-11 Homo sapiens genomic clone RPCI-11-66I23, | Homo sapiens | 41,016 | 20-Apr-99 |
| | | | | | genomic survey sequence. | | | |
| | | GB_HTG2:AC000016 | 194000 | AC000016 | Homo sapiens chromosome 4, *** SEQUENCING IN PROGRESS ***, | Homo sapiens | 38,253 | 16-MAY- |
| | | | | | 9 unordered pieces. | | | 1998 |
| | | GB_STS:G53604 | 617 | G53604 | SHGC-86312 Human Homo sapiens STS genomic, sequence tagged site. | Homo sapiens | 41,016 | 25-Jun-99 |
| rxa01265 | | | | | | | | |
| rxa01274 | 1218 | GB_PAT:E15823 | 2323 | E15823 | DNA encoding cell surface protein from Corynebacterium ammoniagenes. | Corynebacterium | 52,523 | 28-Jul-99 |
| | | | | | | ammoniagenes | | |
| | | GB_HTG3:AF182108 | 167065 | AF182108 | Homo sapiens chromosome 8 clone BAC R-11N9 map 8p12.8, | Homo sapiens | 35,377 | 08-OCT- |
| | | | | | ***SEQUENCING IN PROGRESS ***, in unordered pieces. | | | 1999 |
| | | GB_HTG3:AF182108 | 167065 | AF182108 | Homo sapiens chromosome 8 clone BAC R-11N9 map 8p12.8, | Homo sapiens | 35,377 | 08-OCT- |
| | | | | | ***SEQUENCING IN PROGRESS ***, in unordered pieces. | | | 1999 |
| rxa01278 | 2250 | GB_BA1:MLB1790G | 37617 | Z14314 | M. leprae genes rplL, rpoB, rpoC, end, rpsL, rpsG, efg, tuf, rpsJ, rplC for | Mycobacterium leprae | 70,031 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, elongation | | | |
| | | | | | factor G, elongation factor Tu, ribosomal protein S10, ribosomal protein L3 and | | | |
| | | | | | mkl gene. | | | |
| | | GB_BA1:MTV040 | 15100 | AL021943 | Mycobacterium tuberculosis H37Rv complete genome; segment 33/162. | Mycobacterium tuberculosis | 70,704 | 17-Jun-98 |
| | | GB_BA1:ATFUSATUF | 3412 | X99673 | A. tumefaciens fusA & tufA genes. | Agrobacterium tumefaciens | 64,042 | 11-Nov-96 |
| rxa01283 | 1316 | GB_BA1:MLB1790G | 37617 | Z14314 | M. leprae genes rplL, rpoB, rpoC, end, rpsL, rpsG, efg, tuf, rpsJ, rplC for | Mycobacterium leprae | 65,865 | 11-Feb-93 |
| | | | | | ribosomal protein L7, RNA polymerase beta subunit, RNA polymerase beta′ | | | |
| | | | | | subunit, endonuclease, ribosomal protein S7, ribosomal protein S12, elongation | | | |
| | | | | | factor G, elongation factor Tu, ribosomal protein S10, ribosomal protein L3 and | | | |
| | | | | | mkl gene. | | | |
| | | GB_BA1:MTCI376 | 19770 | Z95972 | Mycobacterium tuberculosis H37Rv complete genome; segment 32/162. | Mycobacterium tuberculosis | 64,633 | 17-Jun-98 |
| | | GB_BA2:ECOUW89 | 176195 | U00006 | E. coli chromosomal region from 89.2 to 92.8 minutes. | Escherichia coli | 46,615 | 17-DEC- |
| | | | | | | | | 1993 |
| rxa01284 | 667 | GB_BA1:CGTUF | 1191 | X77034 | C. glutamicum tuf gene for elongation factor Tu. | Corynebacterium glutamicum | 100,000 | 27-OCT- |
| | | | | | | | | 1994 |
| | | GB_BA1:MTCY210 | 36804 | Z84395 | Mycobacterium tuberculosis H37Rv complete genome; segment 34/162 | Mycobacterium tuberculosis | 74,622 | 17-Jun-98 |
| | | GB_BA1:MSGY42 | 36526 | AD000005 | Mycobacterium tuberculosis sequence from clone y42. | Mycobacterium tuberculosis | 37,419 | 03-DEC- |
| | | | | | | | | 1996 |
| rxa01327 | 267 | GB_SY:SCU53587 | 4546 | U53587 | Artificial Corynebacterium glutamicum IS1207-derived transposon transposase | synthetic construct | 60,674 | 06-MAY- |
| | | | | | genes, complete cds, and 3′5″-aminoglycoside phosphotransferase (aphA-3) gene, | | | 1996 |
| | | | | | complete cds. | | | |
| | | GB_BA1:BLIS13869 | 1840 | Z66534 | B. lactofermentum IS13869 DNA and transposase gene. | Corynebacterium glutamicum | 62,172 | 16-Jul-96 |
| | | EM_PAT:E10419 | 1469 | E10419 | Insertion sequence derived from C. glutamicum. | Corynebacterium glutamicum | 60,674 | 08-OCT- |
| | | | | | | | | 1997 (Rel. |
| | | | | | | | | 52, Created) |
| rxa01328 | 498 | GB_BA1:BLIS13869 | 1840 | Z66534 | B. lactofermentum IS13869 DNA and transposase gene. | Corynebacterium glutamicum | 73,038 | 16-Jul-96 |
| | | GB_SY:SCU53587 | 4546 | U53587 | Artificial Corynebacterium glutamicum IS1207-derived transposon transposase | synthetic construct | 68,813 | 06-MAY- |
| | | | | | genes, complete cds, and 3′5″-aminoglycoside phosphotransferase (aphA-3) gene, | | | 1996 |
| | | | | | complete cds. | | | |
| | | GB_PAT:I43826 | 1452 | I43826 | Sequence 1 from patent U.S. Pat. No. 5633154. | Unknown. | 69,014 | 07-OCT- |
| | | | | | | | | 1997 |
| rxa01329 | 414 | GB_BA1:BLIS13869 | 1840 | Z66534 | B. lactofermentum IS13869 DNA and transposase gene. | Corynebacterium glutamicum | 73,966 | 16-Jul-96 |
| | | GB_PAT:E12758 | 1453 | E12758 | DNA encoding Brevibacterium transposase. | Corynebacterium glutamicum | 73,020 | 24-Jun-98 |
| | | GB_PAT:I33166 | 1453 | I33166 | Sequence 1 from patent U.S. Pat. No. 5591577. | Unknown. | 73,020 | 6-Feb-97 |
| rxa01344 | 2647 | GB_BA1:MSU24494 | 3752 | U24494 | Mycobacterium smegmatis DNA polymerase (rpoB) gene, complete cds. | Mycobacterium smegmatis | 73,086 | 07-MAR- |
| | | | | | | | | 1996 |
| | | GB_BA1:MTCI376 | 19770 | Z95972 | Mycobacterium tuberculosis H37Rv complete genome; segment 32/162. | Mycobacterium tuberculosis | 71,385 | 17-Jun-98 |
| | | GB_BA1:MSGRPOB | 5084 | L27989 | Mycobacterium tuberculosis RNA polymerase beta-subunit (rpoB) gene, complete | Mycobacterium tuberculosis | 71,429 | 13-Sep-94 |
| | | | | | cds and RNA polymerase beta′-subunit rpoC gene, partial cds. | | | |
| rxa01355 | 909 | GB_HTG4:AC009135 | 168607 | AC009135 | Homo sapiens chromosome 16 clone RPCI-11_509E10, *** SEQUENCING IN | Homo sapiens | 37,156 | 31-OCT- |
| | | | | | PROGRESS ***, 231 unordered pieces. | | | 1999 |
| | | GB_HTG4:AC009135 | 168607 | AC009135 | Homo sapiens chromosome 16 clone RPCI-11_509E10, *** SEQUENCING IN | Homo sapiens | 37,156 | 31-OCT- |
| | | | | | PROGRESS ***, 231 unordered pieces. | | | 1999 |
| | | GB_BA1:PFLEPALEP | 1391 | X56466 | P. fluorescens lepA (partial) and lep gene for leader peptidase 1. | Pseudomonas fluorescens | 44,023 | 5-Feb-92 |
| rxa01387 | 469 | GB_BA1:MLB1790G | 37617 | Z14314 | M. leprae genes rplL, rpoB, rpoC, end, rpsL, rpsG, efg, tuf, rpsJ, rplC for | Mycobacterium leprae | 71,429 | 11-Feb-93 |
| | | | | | ribosomal protein L7, RNA polymerase beta subunit, RNA polymerase | | | |
| | | | | | beta′ subunit, endonuclease, ribosomal protein S7, ribosomal | | | |
| | | | | | protein S12, elongation factor G, elongation factor Tu, ribosomal | | | |
| | | | | | protein S10, ribosomal protein L3 and mkl gene. | | | |
| | | GB_BA1:MTCI376 | 19770 | Z95972 | Mycobacterium tuberculosis H37Rv complete genome; segment 32/162. | Mycobacterium tuberculosis | 73,176 | 17-Jun-98 |
| | | GB_BA1:BSUB0001 | 213080 | Z99104 | Bacillus subtilis complete genome (section 1 of 21): from 1 to 213080. | Bacillus subtilis | 63,853 | 26-Nov-97 |
| rxa01388 | 255 | GB_HTG2:HS676J13 | 117045 | AL034347 | Homo sapiens chromosome 6 clone RP4-676J13 map q14, *** | Homo sapiens | 36,863 | 03-DEC- |
| | | | | | SEQUENCING IN PROGRESS ***, in unordered pieces. | | | 1999 |
| | | GB_HTG2:HS676J13 | 117045 | AL034347 | Homo sapiens chromosome 6 clone RP4-676J13 map q14, *** | Homo sapiens | 36,863 | 03-DEC- |
| | | | | | SEQUENCING IN PROGRESS ***, in unordered pieces. | | | 1999 |
| | | GB_HTG2:HS676J13 | 117045 | AL034347 | Homo sapiens chromosome 6 clone RP4-676J13 map q14, *** | Homo sapiens | 29,804 | 03-DEC- |
| | | | | | SEQUENCING IN PROGRESS ***, in unordered pieces. | | | 1999 |
| rxa01398 | 659 | GB_BA1:MTV012 | 70287 | AL021287 | Mycobacterium tuberculosis H37Rv complete genome; segment 132/162. | Mycobacterium tuberculosis | 36,547 | 23-Jun-99 |
| | | GB_BA1:S70345 | 5077 | S70345 | SpaA = endocarditis immunodominant antigen [Streptococcus sobrinus, MUCOB | Streptococcus sobrinus | 35,139 | 23-Sep-94 |
| | | | | | 263, Genomic, 5077 nt]. | | | |
| | | GB_BA1:STRPAGA | 5100 | D90354 | S. sobrinus pag gene for surface protein antigen (PAg). | Streptococcus sobrinus | 35,604 | 7-Feb-99 |
| rxa01431 | 444 | GB_BA2:AE001648 | 13965 | AE001648 | Chlamydia pneumoniae section 64 of 103 of the complete genome. | Chlamydophila pneumoniae | 44,218 | 08-MAR- |
| | | | | | | | | 1999 |
| | | GB_BA2:AE001648 | 13965 | AE001648 | Chlamydia pneumoniae section 64 of 103 of the complete genome. | Chlamydophila pneumoniae | 35,520 | 08-MAR- |
| | | | | | | | | 1999 |
| rxa01432 | 1074 | GB_BA1:MSGY367 | 35336 | AD000008 | Mycobacterium tuberculosis sequence from clone y367. | Mycobacterium tuberculosis | 37,869 | 03-DEC- |
| | | | | | | | | 1996 |
| | | GB_BA1:MTV028 | 11381 | AL021426 | Mycobacterium tuberculosis H37Rv complete genome; segment 162/162. | Mycobacterium tuberculosis | 61,891 | 17-Jun-98 |
| | | GB_BA2:AF023161 | 1775 | AF023161 | Mycobacterium smegmatis thioredoxin reductase (trxB) and thioredoxin (trxA) | Mycobacterium smegmatis | 64,105 | 13-OCT- |
| | | | | | genes, complete cds. | | | 1997 |
| rxa01433 | 726 | GB_BA2:AF105341 | 3010 | AF105341 | Listeria monocytogenes threonine dehydratase (thd1) gene, partial cds; alpha | Listeria monocytogenes | 36,254 | 04-MAR- |
| | | | | | acetolactate decarboxylase gene, complete cds; and pyrimidine nucleoside | | | 1999 |
| | | | | | phosphorylase (pdp1) gene, partial cds. | | | |
| | | GB_BA2:AF105341 | 3010 | AF105341 | Listeria monocytogenes threonine dehydratase (thd1) gene, partial cds; alpha | Listeria monocytogenes | 35,303 | 04-MAR- |
| | | | | | acetolactate decarboxylase gene, complete cds; and pyrimidine nucleoside | | | 1999 |
| | | | | | phosphorylase (pdp1) gene, partial cds. | | | |
| rxa01443 | 954 | GB_BA1:CGISABL | 1290 | X69104 | C. glutamicum IS3 related insertion element. | Corynebacterium glutamicum | 72,823 | 9-Aug-95 |
| | | GB_PAT:I33168 | 1279 | I33168 | Sequence 4 from patent U.S. Pat. No. 5591577. | Unknown. | 72,293 | 6-Feb-97 |
| | | GB_PAT:E12760 | 1279 | E12760 | DNA encoding Brevibacterium transposase. | Corynebacterium glutamicum | 72,293 | 24-Jun-98 |
| rxa01444 | 390 | GB_BA1:CGISABL | 1290 | X69104 | C. glutamicum IS3 related insertion element. | Corynebacterium glutamicum | 69,034 | 9-Aug-95 |
| | | GB_PAT:E12760 | 1279 | E12760 | DNA encoding Brevibacterium transposase. | Corynebacterium glutamicum | 69,318 | 24-Jun-98 |
| | | GB_PAT:I33168 | 1279 | I33168 | Sequence 4 from patent U.S. Pat. No. 5591577. | Unknown. | 69,318 | 6-Feb-97 |
| rxa01449 | 1141 | GB_HTG1:CEY1A5 | 196643 | AL008872 | Caenorhabditis elegans chromosome III clone Y1A5, *** SEQUENCING IN | Caenorhabditis elegans | 36,208 | 9-Nov-97 |
| | | | | | PROGRESS ***, in unordered pieces. | | | |
| | | GB_HTG1:CEY1A5 | 196643 | AL008872 | Caenorhabditis elegans chromosome III clone Y1A5, *** SEQUENCING IN | Caenorhabditis elegans | 36,208 | 9-Nov-97 |
| | | | | | PROGRESS ***, in unordered pieces. | | | |
| | | GB_IN1:PFMAL3P4 | 113899 | AL008970 | Plasmodium falciparum MAL3P4, complete sequence. | Plasmodium falciparum | 33,333 | 28-Jul-99 |
| rxa01490 | 1014 | GB_BA1:MTV002 | 56414 | AL008967 | Mycobacterium tuberculosis H37Rv complete genome; segment 122/162. | Mycobacterium tuberculosis | 36,436 | 17-Jun-98 |
| | | GB_BA1:SC9F2 | 11908 | AL035559 | Streptomyces coelicolor cosmid 9F2. | Streptomyces coelicolor | 36,774 | 25-Feb-99 |
| | | GB_BA1:SPSNBCGEN | 22449 | X98690 | S. pristinaespiralis snbC and snbDE genes. | Streptomyces pristinaespiralis | 41,509 | 24-MAR- |
| | | | | | | | | 1997 |
| rxa01493 | 1434 | GB_HTG3:AC009583 | 172341 | AC009583 | Homo sapiens chromosome 4 clone 158_C_21 map 4, *** SEQUENCING IN | Homo sapiens | 34,102 | 29-Sep-99 |
| | | | | | PROGRESS ***, 17 unordered pieces. | | | |
| | | GB_HTG3:AC009583 | 172341 | AC009583 | Homo sapiens chromosome 4 clone 158_C_21 map 4, *** SEQUENCING IN | Homo sapiens | 34,102 | 29-Sep-99 |
| | | | | | PROGRESS ***, 17 unordered pieces. | | | |
| | | GB_HTG3:AC009583 | 172341 | AC009583 | Homo sapiens chromosome 4 clone 158_C_21 map 4, *** SEQUENCING IN | Homo sapiens | 35,133 | 29-Sep-99 |
| | | | | | PROGRESS ***, 17 unordered pieces. | | | |
| rxa01496 | 3135 | GB_BA1:MTCY16B7 | 43430 | Z81331 | Mycobacterium tuberculosis H37Rv complete genome; segment 123/162. | Mycobacterium tuberculosis | 39,391 | 17-Jun-98 |
| | | GB_BA1:MSGY414A | 40121 | AD000007 | Mycobacterium tuberculosis sequence from clone y414a. | Mycobacterium tuberculosis | 60,308 | 03-DEC- |
| | | | | | | | | 1996 |
| | | GB_BA1:MLCB596 | 38426 | AL035472 | Mycobacterium leprae cosmid B596. | Mycobacterium leprae | 57,989 | 27-Aug-99 |
| rxa01522 | 1701 | GB_BA2:RHMGLTX | 4119 | M27221 | Sinorhizobium meliloti glutamyl-tRNA synthetase (gltX) and lysyl-tRNA | Sinorhizobium meliloti | 49,669 | 11-Sep-98 |
| | | | | | synthetase (lysS) genes, complete cds. | | | |
| | | GB_BA1:MTCY06H11 | 38000 | Z85982 | Mycobacterium tuberculosis H37Rv complete genome; segment 73/162. | Mycobacterium tuberculosis | 38,152 | 17-Jun-98 |
| | | GB_EST8:AA002902 | 396 | AA002902 | mg38a12.r1 Soares mouse embryo NbME13.5 14.5 Mus musculus cDNA clone | Mus musculus | 42,333 | 19-Jul-96 |
| | | | | | IMAGE: 426046 5′, mRNA sequence. | | | |
| rxa01556 | 872 | GB_PR2:HSU73633 | 42845 | U73633 | Human chromosome 11 146h12 cosmid, complete sequence. | Homo sapiens | 37,412 | 19-Jun-97 |
| | | GB_RO:MMU70209 | 14141 | U70209 | Mus musculus polycystic kidney disease 1 protein (Pkd1) mRNA, complete cds. | Mus musculus | 42,536 | 31-MAY- |
| | | | | | | | 1997 | |
| | | GB_HTG2:AC007903 | 170591 | AC007903 | Homo sapiens chromosome 18 clone 563_I_8 map 18, *** SEQUENCING IN | Homo sapiens | 34,868 | 23-Jun-99 |
| | | | | | PROGRESS ***, 6 unordered pieces. | | | |
| rxa01558 | 1332 | GB_BA1:MTCY227 | 35946 | Z77724 | Mycobacterium tuberculosis H37Rv complete genome; segment 114/162. | Mycobacterium tuberculosis | 38,567 | 17-Jun-98 |
| | | GB_BA1:MLCB1259 | 38807 | AL023591 | Mycobacterium leprae cosmid B1259. | Mycobacterium leprae | 53,364 | 27-Aug-99 |
| | | GB_BA1:U00011 | 40429 | U00011 | Mycobacterium leprae cosmid B1177. | Mycobacterium leprae | 38,498 | 01-MAR- |
| | | | | | | | 1994 | |
| rxa01559 | 1965 | GB_BA1:MTCY227 | 35946 | Z77724 | Mycobacterium tuberculosis H37Rv complete genome; segment 114/162. | Mycobacterium tuberculosis | 37,945 | 17-Jun-98 |
| | | GB_BA1:MLCB1259 | 38807 | AL023591 | Mycobacterium leprae cosmid B1259. | Mycobacterium leprae | 51,117 | 27-Aug-99 |
| | | GB_BA1:U00011 | 40429 | U00011 | Mycobacterium leprae cosmid B1177. | Mycobacterium leprae | 37,513 | 01-MAR- |
| | | | | | | | 1994 | |
| rxa01582 | 1212 | GB_BA1:MTCY06H11 | 38000 | Z85982 | Mycobacterium tuberculosis H37Rv complete genome; segment 73/162. | Mycobacterium tuberculosis | 60,249 | 17-Jun-98 |
| | | GB_BA1:MSGB1133CS | 42106 | L78811 | Mycobacterium leprae cosmid B1133 DNA sequence. | Mycobacterium leprae | 58,547 | 15-Jun-96 |
| | | GB_BA1:SCI35 | 40909 | AL031541 | Streptomyces coelicolor cosmid I35. | Streptomyces coelicolor | 37,479 | 9-Sep-98 |
| rxa01583 | 2466 | GB_BA1:MTV004 | 69350 | AL009198 | Mycobacterium tuberculosis H37Rv complete genome; segment 144/162. | Mycobacterium tuberculosis | 39,373 | 18-Jun-98 |
| | | GB_GSS8:AQ077749 | 538 | AQ077749 | CIT-HSP-2367E24.TR CIT-HSP Homo sapiens genomic clone 2367E24, genomic | Homo sapiens | 36,989 | 20-Aug-98 |
| | | | | | survey sequence. | | | |
| | | GB_BA1:MTV004 | 69350 | AL009198 | Mycobacterium tuberculosis H37Rv complete genome; segment 144/162. | Mycobacterium tuberculosis | 39,220 | 18-Jun-98 |
| rxa01596 | 1902 | GB_BA1:SCI51 | 40745 | AL109848 | Streptomyces coelicolor cosmid I51. | Streptomyces coelicolor A3(2) | 38,388 | 16-Aug-99 |
| | | GB_BA1:MTCI125 | 37432 | Z98268 | Mycobacterium tuberculosis H37Rv complete genome; segment 76/162. | Mycobacterium tuberculosis | 53,052 | 17-Jun-98 |
| | | GB_BA1:MTHYPROT | 2544 | X98295 | M. tuberculosis TlyA gene. | Mycobacterium tuberculosis | 49,393 | 2-Jun-98 |
| rxa01601 | 1035 | GB_BA1:MTCI125 | 37432 | Z98268 | Mycobacterium tuberculosis H37Rv complete genome; segment 76/162. | Mycobacterium tuberculosis | 54,801 | 17-Jun-98 |
| | | GB_BA1:MLCB1351 | 38936 | Z95117 | Mycobacterium leprae cosmid B1351. | Mycobacterium leprae | 39,577 | 24-Jun-97 |
| | | GB_BA1:U00021 | 39193 | U00021 | Mycobacterium leprae cosmid L247. | Mycobacterium leprae | 39,476 | 29-Sep-94 |
| rxa01613 | 1338 | GB_BA1:MTCY24A1 | 20270 | Z95207 | Mycobacterium tuberculosis H37Rv complete genome; segment 124/162. | Mycobacterium tuberculosis | 52,216 | 17-Jun-98 |
| | | GB_BA1:AF002193 | 1812 | AF002193 | Mycobacterium tuberculosis glutathione reductase homolog (gorA) gene, | Mycobacterium tuberculosis | 52,216 | 18-Jul-97 |
| | | | | | complete cds. | | | |
| | | GB_HTG3:AC008675 | 206439 | AC008675 | Homo sapiens chromosome 5 clone CIT978SKB_45I8, *** SEQUENCING IN | Homo sapiens | 36,145 | 3-Aug-99 |
| | | | | | PROGRESS ***, 43 unordered pieces. | | | |
| rxa01621 | 1563 | GB_BA1:MTY15F10 | 38204 | Z94121 | Mycobacterium tuberculosis H37Rv complete genome; segment 161/162. | Mycobacterium tuberculosis | 36,776 | 17-Jun-98 |
| | | GB_BA1:MSGY367 | 35336 | AD000008 | Mycobacterium tuberculosis sequence from clone y367. | Mycobacterium tuberculosis | 60,525 | 03-DEC- |
| | | | | | | | 1996 | |
| | | GB_BA1:MTY15F10 | 38204 | Z94121 | Mycobacterium tuberculosis H37Rv complete genome; segment 161/162. | Mycobacterium tuberculosis | 36,288 | 17-Jun-98 |
| rxa01648 | 492 | GB_BA1:CGISABL | 1290 | X69104 | C. glutamicum IS3 related insertion element. | Corynebacterium glutamicum | 76,483 | 9-Aug-95 |
| | | GB_PAT:AR038104 | 1279 | AR038104 | Sequence 9 from patent U.S. Pat. No. 5804414. | Unknown. | 75,574 | 29-Sep-99 |
| | | GB_PAT:E12760 | 1279 | E12760 | DNA encoding Brevibacterium transposase. | Corynebacterium glutamicum | 75,574 | 24-Jun-98 |
| rxa01649 | 543 | GB_BA1:CGISABL | 1290 | X69104 | C. glutamicum IS3 related insertion element. | Corynebacterium glutamicum | 67,978 | 9-Aug-95 |
| | | GB_PAT:AR038104 | 1279 | AR038104 | Sequence 9 from patent U.S. Pat. No. 5804414. | Unknown. | 67,857 | 29-Sep-99 |
| | | GB_PAT:E12760 | 1279 | E12760 | DNA encoding Brevibacterium transposase. | Corynebacterium glutamicum | 67,857 | 24-Jun-98 |
| rxa01650 | 237 | GB_PL2:SPAC17A2 | 36642 | Z99292 | S. pombe chromosome I cosmid c17A2. | Schizosaccharomyces pombe | 42,241 | 22-Jul-99 |
| | | GB_PL2:SPAC17A2 | 36642 | Z99292 | S. pombe chromosome I cosmid c17A2. | Schizosaccharomyces pombe | 33,766 | 22-Jul-99 |
| | | GB_PL1:SCYDR012W | 2732 | Z74308 | S. cerevisiae chromosome IV reading frame ORF YDR012w. | Saccharomyces cerevisiae | 30,804 | 12-Aug-98 |
| rxa01651 | 258 | GB_BA1:CGISABL | 1290 | X69104 | C. glutamicum IS3 related insertion element. | Corynebacterium glutamicum | 69,643 | 9-Aug-95 |
| | | GB_PAT:AR038104 | 1279 | AR038104 | Sequence 9 from patent U.S. Pat. No. 5804414. | Unknown. | 67,265 | 29-Sep-99 |
| | | GB_PAT:I33168 | 1279 | I33168 | Sequence 4 from patent U.S. Pat. No. 5591577. | Unknown. | 67,265 | 6-Feb-97 |
| rxa01670 | 930 | GB_BA2:SMU56906 | 3303 | U56906 | Serratia marcescens DNA gyrase (gyrA) gene, complete cds. | Serratia marcescens | 36,186 | 7-Jan-98 |
| | | GB_BA1:D90902 | 122056 | D90902 | Synechocystis sp. PCC6803 complete genome, 4/27, 402290-524345. | Synechocystis sp. | 37,814 | 7-Feb-99 |
| | | GB_HTG2:HSDJ816K9 | 144277 | AL117349 | Homo sapiens chromosome 1 clone RP4-816K9, *** SEQUENCING IN | Homo sapiens | 41,759 | 30-Nov-99 |
| | | | | | PROGRESS ***, in unordered pieces. | | | |
| rxa01680 | | | | | | | | |
| rxa01704 | 1100 | GB_HTG2:AF129075 | 195012 | AF129075 | Homo sapiens chromosome 21 clone J12100; E0479 map 21q22.1, | Homo sapiens | 40,187 | 03-MAR- |
| | | | | | ***SEQUENCING IN PROGRESS ***, in ordered pieces. | | 1999 | |
| | | GB_HTG2:AF129075 | 195012 | AF129075 | Homo sapiens chromosome 21 clone J12100; E0479 map 21q22.1, | Homo sapiens | 40,187 | 03-MAR- |
| | | | | | ***SEQUENCING IN PROGRESS ***, in ordered pieces. | | 1999 | |
| | | GB_HTG2:AC007271 | 184269 | AC007271 | Homo sapiens clone NH0004B12, *** SEQUENCING IN PROGRESS ***, 2 | Homo sapiens | 38,667 | 16-Apr-99 |
| | | | | | unordered pieces. | | | |
| rxa01710 | 531 | GB_BA1:MTCY441 | 35187 | Z80225 | Mycobacterium tuberculosis H37Rv complete genome; segment 118/162. | Mycobacterium tuberculosis | 56,309 | 18-Jun-98 |
| | | GB_EST16:AA540562 | 695 | AA540562 | LD20282.5prime LD Drosophila melanogaster embryo BlueScript Drosophila | Drosophila melanogaster | 51,357 | 28-Nov-98 |
| | | | | | melanogaster cDNA clone LD20282 5prime, mRNA sequence. | | | |
| | | GB_EST37:AI944677 | 580 | AI944677 | bs04b04.y1 Drosophila melanogaster adult testis library Drosophila melanogaster | Drosophila melanogaster | 50,728 | 17-Aug-99 |
| | | | | | cDNA clone bs04b04 5′, mRNA sequence. | | | |
| rxa01724 | 1343 | GB_BA1:MLU15186 | 36241 | U15186 | Mycobacterium leprae cosmid L471. | Mycobacterium leprae | 37,412 | 09-MAR- |
| | | | | | | | 1995 | |
| | | GB_BA1:MTCY373 | 35516 | Z73419 | Mycobacterium tuberculosis H37Rv complete genome; segment 57/162. | Mycobacterium tuberculosis | 47,819 | 17-Jun-98 |
| | | GB_HTG2:AC007608 | 170057 | AC007608 | Homo sapiens chromosome 16 clone 401P9, *** SEQUENCING IN | Homo sapiens | 37,236 | 20-MAY- |
| | | | | | PROGRESS***, 59 unordered pieces. | | 1999 | |
| rxa01725 | 330 | GB_BA1:MTCY373 | 35516 | Z73419 | Mycobacterium tuberculosis H37Rv complete genome; segment 57/162. | Mycobacterium tuberculosis | 75,610 | 17-Jun-98 |
| | | GB_BA1:MLU15186 | 36241 | U15186 | Mycobacterium leprae cosmid L471. | Mycobacterium leprae | 39,355 | 09-MAR- |
| | | | | | | | 1995 | |
| | | GB_BA1:PSERHO | 1479 | L27278 | Pseudomonas fluorescens rho gene, complete cds. | Pseudomonas fluorescens | 63,303 | 9-Jan-95 |
| rxa01726 | 696 | GB_BA1:MTCY373 | 35516 | Z73419 | Mycobacterium tuberculosis H37Rv complete genome; segment 57/162. | Mycobacterium tuberculosis | 72,899 | 17-Jun-98 |
| | | GB_BA1:MLU15186 | 36241 | U15186 | Mycobacterium leprae cosmid L471. | Mycobacterium leprae | 37,500 | 09-MAR- |
| | | | | | | | 1995 | |
| | | GB_BA1:SLRHOGENE | 2986 | X95444 | S. lividans Rho gene. | Streptomyces lividans | 69,065 | 1-Feb-96 |
| rxa01730 | 1804 | GB_BA1:MTCY227 | 35946 | Z77724 | Mycobacterium tuberculosis H37Rv complete genome; segment 114/162. | Mycobacterium tuberculosis | 39,943 | 17-Jun-98 |
| | | GB_BA1:MLCB1259 | 38807 | AL023591 | Mycobacterium leprae cosmid B1259. | Mycobacterium leprae | 65,120 | 27-Aug-99 |
| | | GB_BA2:S82268 | 2209 | S82268 | Mycobacterium leprae ASPS and antigen T5 genes, complete cds. | Mycobacterium leprae | 40,715 | 22-Jul-98 |
| rxa01733 | 1274 | GB_BA1:MSORIREP | 10430 | X92503 | M. smegmatis origin of replication and genes rpmH, dnaA, dnaN, gnd, recF, | Mycobacterium smegmatis | 52,740 | 26-Aug-97 |
| | | | | | gyrB, gyrA. | | | |
| | | GB_BA1:MSGYRBA | 6000 | X94224 | M. smegmatis gyrB and gyrA genes. | Mycobacterium smegmatis | 52,277 | 12-Feb-97 |
| | | GB_HTG4:AC010890 | 175554 | AC010890 | Homo sapiens chromosome unknown clone NH0449L24, WORKING DRAFT | Homo sapiens | 36,601 | 29-OCT- |
| | | | | | SEQUENCE, in unordered pieces. | | 1999 | |
| rxa01736 | 2891 | GB_BA1:MTV014 | 58280 | AL021646 | Mycobacterium tuberculosis H37Rv complete genome; segment 137/162. | Mycobacterium tuberculosis | 38,918 | 18-Jun-98 |
| | | GB_PL2:AF156928 | 2290 | AF156928 | Candida albicans folylpolyglutamate synthetase (fpgs) gene, complete cds. | Candida albicans | 34,894 | 22-Jun-99 |
| | | GB_GSS12:AQ421204 | 483 | AQ421204 | RPCI-11-167B4.TJ RPCI-11 Homo sapiens genomic clone RPCI-11-167B4, | Homo sapiens | 39,085 | 23-MAR- |
| | | | | | genomic survey sequence. | | | 1999 |
| rxa01737 | 1182 | GB_BA1:SCGD3 | 33779 | AL096822 | Streptomyces coelicolor cosmid GD3. | Streptomyces coelicolor | 38,054 | 8-Jul-99 |
| | | GB_HTG1:CNS01DSB | 222193 | AL121768 | Homo sapiens chromosome 14 clone R-976B16, *** SEQUENCING IN | Homo sapiens | 35,147 | 05-OCT- |
| | | | | | PROGRESS ***, in ordered pieces. | | | 1999 |
| | | GB_HTG1:CNS01DSB | 222193 | AL121768 | Homo sapiens chromosome 14 clone R-976B16, *** SEQUENCING IN | Homo sapiens | 35,147 | 05-OCT- |
| | | | | | PROGRESS ***, in ordered pieces. | | | 1999 |
| rxa01784 | 705 | GB_BA2:AF121000 | 19751 | AF121000 | Corynebacterium glutamicum strain 22243 R-plasmid pAG1, complete sequence. | Corynebacterium glutamicum | 36,270 | 14-Apr-99 |
| | | GB_BA1:FVBPOAD2A | 45519 | D26094 | Flavobacterium sp. plasmid pOAD2 DNA, whole sequence. | Flavobacterium sp. | 38,450 | 6-Feb-99 |
| | | GB_BA1:FVBPOAD2A | 45519 | D26094 | Flavobacterium sp. plasmid pOAD2 DNA, whole sequence. | Flavobacterium sp. | 59,052 | 6-Feb-99 |
| rxa01798 | 373 | GB_IN1:AB018440 | 13738 | AB018440 | Echinococcus multilocularis mitochondrial DNA, complete genome. | Mitochondrion Echinococcus | 34,877 | 28-OCT- |
| | | GB_BA1:SSU82227 | 8313 | U82227 | Sulfolobus solfataricus leucyl-tRNA synthetase (leuS) gene, partial cds, histidine | Sulfolobus solfataricus | 40,166 | 14-Jul-97 |
| | | | | | biosynthesis operon hisCGABdFDEHI, (hisC, hisG, hisBd, hisF, hisD, hisE, hisH | | | |
| | | | | | and hisI) genes, complete cds and seryl-tRNA synthetase (serS) gene, partial cds. | | | |
| | | GB_BA1:SSU82227 | 8313 | U82227 | Sulfolobus solfataricus leucyl-tRNA synthetase (leuS) gene, partial cds, histidine | Sulfolobus solfataricus | 33,989 | 14-Jul-97 |
| | | | | | biosynthesis operon hisCGABdFDEHI, (hisC, hisG, hisBd, hisF, hisD, hisE, hisH | | | |
| | | | | | and hisI) genes, complete cds and seryl-tRNA synthetase (serS) gene, partial cds. | | | |
| rxa01818 | 1110 | GB_IN1:CEF08G5 | 32784 | Z70682 | Caenorhabditis elegans cosmid F08G5, complete sequence. | Caenorhabditis elegans | 35,032 | 23-Jul-99 |
| | | GB_HTG2:AC008029 | 123186 | AC008029 | Drosophila melanogaster chromosome 3 clone BACR01C11 (D819) RPCI-98 | Drosophila melanogaster | 35,197 | 2-Aug-99 |
| | | | | | 01.C.11 map 84D-84D strain y; cn bw sp, *** SEQUENCING IN | | | |
| | | | | | PROGRESS***, 92 unordered pieces. | | | |
| | | GB_HTG2:AC008029 | 123186 | AC008029 | Drosophila melanogaster chromosome 3 clone BACR01C11 (D819) RPCI-98 | Drosophila melanogaster | 35,197 | 2-Aug-99 |
| | | | | | 01.C.11 map 84D-84D strain y; cn bw sp, *** SEQUENCING IN | | | |
| | | | | | PROGRESS***, 92 unordered pieces. | | | |
| rxa01819 | 570 | GB_BA1:AB023076 | 4953 | AB023076 | Pseudomonas syringae DNA, the left outside of the hrpL homology region, | Pseudomonas syringae | 36,852 | 26-Feb-99 |
| | | | | | strain: KW11. | | | |
| | | GB_BA1:AB023076 | 4953 | AB023076 | Pseudomonas syringae DNA, the left outside of the hrpL homology region, | Pseudomonas syringae | 39,646 | 26-Feb-99 |
| | | | | | strain: KW11. | | | |
| rxa01837 | 900 | GB_BA1:MTCY227 | 35946 | Z77724 | Mycobacterium tuberculosis H37Rv complete genome; segment 114/162. | Mycobacterium tuberculosis | 53,182 | 17-Jun-98 |
| | | GB_HTG2:AC006779 | 119562 | AC006779 | Caenorhabditis elegans clone Y47D7, *** SEQUENCING IN | Caenorhabditis elegans | 34,783 | 25-Feb-99 |
| | | | | | PROGRESS ***, 32 unordered pieces. | | | |
| | | GB_HTG2:AC006779 | 119562 | AC006779 | Caenorhabditis elegans clone Y47D7, *** SEQUENCING IN | Caenorhabditis elegans | 34,783 | 25-Feb-99 |
| | | | | | PROGRESS ***, 32 unordered pieces. | | | |
| rxa01841 | 486 | GB_BA2:AF139249 | 1383 | AF139249 | Actinobacillus actinomycetemcomitans rough | Actinobacillus | 37,395 | 25-MAY- |
| | | | | | colony protein A (rcpA) gene, complete cds. | actinomycetemcomitans | | 1999 |
| | | GB_EST17:C76899 | 603 | C76899 | C76899 Mouse 3.5-dpc blastocyst cDNA Mus musculus cDNA clone J0022E02 3′ | Mus musculus | 44,828 | 25-Jun-98 |
| | | | | | similar to M. musculus DNA for LINE-1 or L1 element, mRNA sequence. | | | |
| | | GB_PR3:U94190 | 6469 | U94190 | Homo sapiens Duo mRNA, complete cds. | Homo sapiens | 38,382 | 04-MAY- |
| | | | | | | | | 1998 |
| rxa01852 | 1410 | GB_BA1:MTCY227 | 35946 | Z77724 | Mycobacterium tuberculosis H37Rv complete genome; segment 114/162. | Mycobacterium tuberculosis | 38,378 | 17-Jun-98 |
| | | GB_BA1:MLCB1259 | 38807 | AL023591 | Mycobacterium leprae cosmid B1259. | Mycobacterium leprae | 59,574 | 27-Aug-99 |
| | | GB_BA1:U00011 | 40429 | U00011 | Mycobacterium leprae cosmid B1177. | Mycobacterium leprae | 37,690 | 01-MAR- |
| | | | | | | | | 1994 |
| rxa01862 | 1329 | GB_BA1:RLDCTA | 5820 | Z11529 | R. leguminosarum dctA gene encoding C4-dicarboxylate permease. | Rhizobium leguminosarum | 39,401 | 23-Sep-92 |
| | | GB_BA1:RLDCTBD | 3360 | X06253 | Rhizobium leguminosarum dctB and dctD genes involved in C4-dicarboxylate | Rhizobium leguminosarum | 39,401 | 12-Sep-93 |
| | | | | | transport. | | | |
| | | GB_BA1:RLDCTA | 5820 | Z11529 | R. leguminosarum dctA gene encoding C4-dicarboxylate permease. | Rhizobium leguminosarum | 39,269 | 23-Sep-92 |
| rxa01863 | 1219 | GB_BA1:BSUB0005 | 208430 | Z99108 | Bacillus subtilis complete genome (section 5 of 21): from 802821 to 1011250. | Bacillus subtilis | 35,673 | 26-Nov-97 |
| | | GB_BA1:D83967 | 22197 | D83967 | Bacillus subtilis genomic DNA, 74 degree region. | Bacillus subtilis | 57,261 | 20-Nov-97 |
| | | GB_BA1:STAATTB | 300 | M20393 | S. aureus bacteriophage phi-11 attachment site (attB). | Staphylococcus aureus | 99,595 | 26-Apr-93 |
| rxa01872 | 928 | GB_GSS15:AQ651661 | 422 | AQ651661 | Sheared DNA-5N18.TR Sheared DNA Trypanosoma brucei genomic clone | Trypanosoma brucei | 42,034 | 22-Jun-99 |
| | | | | | Sheared DNA-5N18, genomic survey sequence. | | | |
| | | GB_GSS15:AQ639444 | 175 | AQ639444 | 927P1-17G6.TV 927P1 Trypanosoma brucei genomic clone 927P1-17G6, | Trypanosoma brucei | 51,786 | 8-Jul-99 |
| | | | | | genomic survey sequence. | | | |
| | | GB_HTG3:AC009919 | 134724 | AC009919 | Homo sapiens clone 115_I_23, LOW-PASS SEQUENCE SAMPLING. | Homo sapiens | 37,222 | 8-Sep-99 |
| rxa01878 | 1002 | GB_HTG1:CEY64F11 | 177748 | Z99776 | Caenorhabditis elegans chromosome IV clone Y64F11, *** SEQUENCING IN | Caenorhabditis elegans | 37,564 | 14-OCT- |
| | | | | | PROGRESS ***, in unordered pieces. | | | 1998 |
| | | GB_HTG1:CEY64F11 | 177748 | Z99776 | Caenorhabditis elegans chromosome IV clone Y64F11, *** SEQUENCING IN | Caenorhabditis elegans | 37,564 | 14-OCT- |
| | | | | | PROGRESS ***, in unordered pieces. | | | 1998 |
| | | GB_HTG1:CEY64F11 | 177748 | Z99776 | Caenorhabditis elegans chromosome IV clone Y64F11, *** SEQUENCING IN | Caenorhabditis elegans | 37,576 | 14-OCT- |
| | | | | | PROGRESS ***, in unordered pieces. | | | 1998 |
| rxa01913 | 948 | GB_BA1:MTCY274 | 39991 | Z74024 | Mycobacterium tuberculosis H37Rv complete genome; segment 126/162. | Mycobacterium tuberculosis | 39,631 | 19-Jun-98 |
| | | GB_BA1:SC2E1 | 38962 | AL023797 | Streptomyces coelicolor cosmid 2E1. | Streptomyces coelicolor | 58,226 | 4-Jun-98 |
| | | GB_BA2:AF130345 | 965 | AF130345 | Streptomyces ramocissimus elongation factor Ts (tsf) gene, complete cds. | Streptomyces ramocissimus | 58,009 | 15-OCT- |
| | | | | | | | | 1999 |
| rxa01938 | 1551 | GB_BA1:MTCY24A1 | 20270 | Z95207 | Mycobacterium tuberculosis H37Rv complete genome; segment 124/162. | Mycobacterium tuberculosis | 38,976 | 17-Jun-98 |
| | | GB_GSS1:CNS00WZY | 720 | AL094252 | Arabidopsis thaliana genome survey sequence SP6 end of BAC T12O8 of TAMU | Arabidopsis thaliana | 54,028 | 28-Jun-99 |
| | | | | | library from strain Columbia of Arabidopsis thaliana, genomic survey sequence. | | | |
| | | GB_PR2:AP000056 | 100000 | AP000056 | Homo sapiens genomic DNA, chromosome 21q22.1, segment 27/28, complete | Homo sapiens | 36,967 | 20-Nov-99 |
| | | | | | sequence. | | | |
| rxa01953 | 504 | GB_BA1:MSGTNP | 2276 | M76495 | Mycobacterium smegmatis insertion element tnpR and tnpA genes, complete cds. | Mycobacterium smegmatis | 38,153 | 26-Apr-96 |
| | | GB_BA2:E12PHEAB | 6164 | M57500 | Plasmid pEST1226 putative transposase (tnpA), catechol 1,2-dioxygenase (pheB), | Plasmid pEST1226 | 56,338 | 21-OCT- |
| | | | | | phenol monooxygenase (pheA), and putative transposase (tnpA) genes, complete | | | 1998 |
| | | | | | cds. | | | |
| | | GB_PR2:HS179N16 | 172048 | Z95152 | Homo sapiens DNA sequence from PAC 179N16 on chromosome 6p21.1-21.33. | Homo sapiens | 34,490 | 23-Nov-99 |
| | | | | | Contains the SAPK4 (MAPK p38delta) gene, and the alternatively spliced SAPK2 | | | |
| | | | | | gene coding for CSaids binding protein CSBP2 and a MAPK p38beta | | | |
| | | | | | LIKE protein. Contains ESTs, STSs and two predicted CpG islands, | | | |
| | | | | | complete sequence. | | | |
| rxa01954 | 963 | GB_BA1:SC4H8 | 15560 | AL020958 | Streptomyces coelicolor cosmid 4H8. | Streptomyces coelicolor | 37,960 | 10-DEC- |
| | | | | | | | | 1997 |
| | | GB_GSS3:B91274 | 183 | B91274 | CIT-HSP-2168G14.TF CIT-HSP Homo sapiens genomic clone 2168G14, | Homo sapiens | 36,066 | 25-Jun-98 |
| | | | | | genomic survey sequence. | | | |
| | | GB_BA1:SC4H8 | 15560 | AL020958 | Streptomyces coelicolor cosmid 4H8. | Streptomyces coelicolor | 39,457 | 10-DEC- |
| | | | | | | | | 1997 |
| rxa01975 | 2019 | GB_BA2:CGU13922 | 4412 | U13922 | Corynebacterium glutamicum putative type II 5-cytosoine methyltransferase | Corynebacterium glutamicum | 99,950 | 3-Feb-98 |
| | | | | | (cgIIM) and putative type II restriction endonuclease (cgIIR) and putative type I | | | |
| | | | | | or type III restriction endonuclease (clgIIR) genes, complete cds. | | | |
| | | GB_BA1:SPSNBCDE | 22449 | Y11548 | S. pristinaespiralis snbC gene & snbDE gene. | Streptomyces pristinaespiralis | 36,657 | 25-Apr-97 |
| | | GB_BA1:SPSNBCGEN | 22449 | X98690 | S. pristinaespiralis snbC and snbDE genes. | Streptomyces pristinaespiralis | 36,657 | 24-MAR- |
| | | | | | | | | 1997 |
| rxa01998 | 831 | GB_BA2:AF121000 | 19751 | AF121000 | Corynebacterium glutamicum strain 22243 R-plasmid pAG1, complete sequence. | Corynebacterium glutamicum | 40,520 | 14-Apr-99 |
| | | GB_BA2:AF121000 | 19751 | AF121000 | Corynebacterium glutamicum strain 22243 R-plasmid pAG1, complete sequence. | Corynebacterium glutamicum | 54,699 | 14-Apr-99 |
| | | GB_BA1:FVBPOAD2A | 45519 | D26094 | Flavobacterium sp. plasmid pOAD2 DNA, whole sequence. | Flavobacterium sp. | 38,562 | 6-Feb-99 |
| rxa02002 | 478 | GB_BA1:STYPRFC | 2140 | D50496 | Salmonella typhimurium gene for peptide release factor 3/RF3, complete cds. | Salmonella typhimurium | 53,289 | 10-Feb-99 |
| | | GB_BA2:U32846 | 11650 | U32846 | Haemophilus influenzae Rd section 161 of 163 of the complete genome. | Haemophilus influenzae Rd | 47,265 | 29-MAY- |
| | | | | | | | | 1998 |
| | | GB_BA2:AF072440 | 4316 | AF072440 | Enterobacter gergoviae GTPase (bipA) gene, partial cds; glutamine synthetase | Enterobacter gergoviae | 37,284 | 30-OCT- |
| | | | | | (glnA) and nitrogen regulatory protein (ntrB) genes, complete cds; and nitrogen | | | 1998 |
| | | | | | regulatory protein (ntrC) gene, partial cds. | | | |
| rxa02015 | 619 | GB_PL2:AF015560 | 2681 | AF015560 | Neurospora crassa RO11 (ro-11) gene, complete cds. | Neurospora crassa | 38,953 | 3-Sep-97 |
| | | GB_GSS13:AQ497173 | 511 | AQ497173 | HS_5193_B2_A10_T7A RPCI-11 Human Male BAC Library Homo sapiens | Homo sapiens | 37,086 | 28-Apr-99 |
| | | | | | genomic clone Plate = 769 Col = 20 Row = B, genomic survey sequence. | | | |
| | | GB_PL1:SPAC27D7 | 35892 | AL009227 | S. pombe chromosome I cosmid c27D7. | Schizosaccharomyces pombe | 39,016 | 25-MAR- |
| | | | | | | | | 1999 |
| rxa02025 | 774 | GB_BA1:ECOUW93 | 338534 | U14003 | Escherichia coli K-12 chromosomal region from 92.8 to 00.1 minutes. | Escherichia coli | 39,108 | 17-Apr-96 |
| | | GB_BA2:AE000493 | 10819 | AE000493 | Escherichia coli K-12 MG1655 section 383 of 400 of the complete genome. | Escherichia coli | 39,108 | 12-Nov-98 |
| | | GB_BA1:ECOPMSR | 1270 | M89992 | Escherichia coli peptide methionine sulfoxide reductase gene, complete cds. | Escherichia coli | 50,329 | 26-Apr-93 |
| rxa02065 | 771 | GB_BA2:MSU87307 | 1520 | U87307 | Mycobacterium smegmatis extracytoplasmic function alternative sigma | Mycobacterium smegmatis | 59,533 | 07-MAY- |
| | | | | | factor (sigE) gene, complete cds. | | | 1997 |
| | | GB_BA1:MTCI61 | 13540 | Z98260 | Mycobacterium tuberculosis H37Rv complete genome; segment 53/162. | Mycobacterium tuberculosis | 57,833 | 17-Jun-98 |
| | | GB_BA2:MTU87242 | 3690 | U87242 | Mycobacterium tuberculosis sigma factor SigE (sigE) and HtrA (htrA) genes, | Mycobacterium tuberculosis | 57,833 | 08-MAY- |
| | | | | | complete cds. | | | 1997 |
| rxa02078 | 981 | GB_BA1:MTCY338 | 29372 | Z74697 | Mycobacterium tuberculosis H37Rv complete genome; segment 127/162. | Mycobacterium tuberculosis | 38,050 | 17-Jun-98 |
| | | GB_BA1:MLCB1243 | 42926 | AL023635 | Mycobacterium leprae cosmid B1243. | Mycobacterium leprae | 53,733 | 27-Aug-99 |
| | | GB_BA1:MSGB1723CS | 38477 | L78825 | Mycobacterium leprae cosmid B1723 DNA sequence. | Mycobacterium leprae | 53,733 | 15-Jun-96 |
| rxa02110 | 741 | GB_EST20:AA894760 | 281 | AA894760 | oj55a09.s1 NCI_CGAP_Kid3 Homo sapiens cDNA clone IMAGE: 1502200 3′, | Homo sapiens | 39,928 | 9-Jun-98 |
| | | | | | mRNA sequence. | | | |
| | | GB_EST38:AL119293 | 323 | AL119293 | DKFZp761B161_r1 761 (synonym: hamy2) Homo sapiens cDNA clone | Homo sapiens | 34,579 | 27-Sep-99 |
| | | | | | DKFZp761B161 5′, mRNA sequence. | | | |
| | | GB_PR3:HSJ1031J8 | 155213 | AL118523 | Human DNA sequence from clone RP5-1031J8 on chromosome 20, complete | Homo sapiens | 32,341 | 03-DEC- |
| | | | | | sequence. | | | 1999 |
| rxa02167 | 1383 | GB_BA1:MTCI125 | 37432 | Z98268 | Mycobacterium tuberculosis H37Rv complete genome; segment 76/162. | Mycobacterium tuberculosis | 63,215 | 17-Jun-98 |
| | | GB_BA1:MLCB1351 | 38936 | Z95117 | Mycobacterium leprae cosmid B1351. | Mycobacterium leprae | 38,240 | 24-Jun-97 |
| | | GB_BA1:U00021 | 39193 | U00021 | Mycobacterium leprae cosmid L247. | Mycobacterium leprae | 37,964 | 29-Sep-94 |
| rxa02174 | 477 | GB_BA1:CGGLTG | 3013 | X66112 | C. glutamicum glt gene for citrate synthase and ORF. | Corynebacterium glutamicum | 100,000 | 17-Feb-95 |
| | | GB_PR4:AF117829 | 320250 | AF117829 | Homo sapiens 8q21.3: RICK gene, complete sequence. | Homo sapiens | 37,528 | 13-Jan-99 |
| | | GB_PR4:AF117829 | 320250 | AF117829 | Homo sapiens 8q21.3: RICK gene, complete sequence. | Homo sapiens | 40,733 | 13-Jan-99 |
| rxa02182 | | | | | | | | |
| rxa02204 | 1383 | GB_BA1:MTCY261 | 27322 | Z97559 | Mycobacterium tuberculosis H37Rv complete genome; segment 95/162. | Mycobacterium tuberculosis | 39,846 | 17-Jun-98 |
| | | GB_BA1:ECU82664 | 139818 | U82664 | Escherichia coli minutes 9 to 11 genomic sequence. | Escherichia coli | 47,528 | 11-Jan-97 |
| | | GB_BA2:AE000158 | 10143 | AE000158 | Escherichia coli K-12 MG1655 section 48 of 400 of the complete genome. | Escherichia coli | 47,528 | 12-Nov-98 |
| rxa02228 | 1026 | GB_HTG2:AC007962 | 172091 | AC007962 | Homo sapiens chromosome 17 clone 2511_J_5 map 17, *** SEQUENCING IN | Homo sapiens | 39,051 | 3-Jul-99 |
| | | | | | PROGRESS ***, 25 unordered pieces. | | | |
| | | GB_HTG2:AC007962 | 172091 | AC007962 | Homo sapiens chromosome 17 clone 2511_J_5 map 17, *** SEQUENCING IN | Homo sapiens | 39,051 | 3-Jul-99 |
| | | | | | PROGRESS ***, 25 unordered pieces. | | | |
| | | GB_HTG3:AC008363 | 131230 | AC008363 | Drosophila melanogaster chromosome 3 clone BACR14H24 (D989) RPCI-98 | Drosophila melanogaster | 31,957 | 3-Aug-99 |
| | | | | | 14.H.24 map 92A-92A strain y; cn bw sp, *** SEQUENCING IN | | | |
| | | | | | PROGRESS***, 91 unordered pieces. | | | |
| rxa02236 | 441 | GB_BA2:MSU75344 | 1458 | U75344 | Mycobacterium smegmatis integration host factor (mIHF) gene, complete cds. | Mycobacterium smegmatis | 63,908 | 4-Aug-98 |
| | | GB_BA1:MTCY21B4 | 39150 | Z80108 | Mycobacterium tuberculosis H37Rv complete genome; segment 62/162. | Mycobacterium tuberculosis | 58,957 | 23-Jun-98 |
| | | GB_BA2:AF077324 | 5228 | AF077324 | Rhodococcus equi strain 103 plasmid RE-VP1 fragment f. | Rhodococcus equi | 40,639 | 5-Nov-98 |
| rxa02242 | 630 | GB_EST30:AI667039 | 548 | AI667039 | fc24h04.y1 Zebrafish WashU MPIMG EST Danio rerio cDNA 5′ similar to | Danio rerio | 46,903 | 18-MAY- |
| | | | | | TR: O93510 O93510 HOMOGENIN.;, mRNA sequence. | | | 1999 |
| | | GB_EST30:AI667039 | 548 | AI667039 | fc24h04.y1 Zebrafish WashU MPIMG EST Danio rerio cDNA 5′ similar to | Danio rerio | 38,445 | 18-MAY- |
| | | | | | TR: O93510 O93510 HOMOGENIN.;, mRNA sequence. | | | 1999 |
| rxa02243 | 1068 | GB_EST8:AA050680 | 515 | AA050680 | mj20f12.r1 Soares mouse embryo NbME13.5 14.5 Mus musculus cDNA clone | Mus musculus | 40,313 | 9-Sep-96 |
| | | | | | IMAGE: 476687 5′, mRNA sequence. | | | |
| | | GB_EST28:AI509997 | 372 | AI509997 | mj20f12.y1 Soares mouse embryo NbME13.5 14.5 Mus musculus cDNA clone | Mus musculus | 40,431 | 12-MAR- |
| | | | | | IMAGE: 476687 5′, mRNA sequence. | | | 1999 |
| | | GB_EST27:AI426148 | 445 | AI426148 | mj20f12.x1 Soares mouse embryo NbME13.5 14.5 Mus musculus cDNA clone | Mus musculus | 45,775 | 09-MAR- |
| | | | | | IMAGE: 476687 3′, mRNA sequence. | | | 1999 |
| rxa02252 | 1544 | GB_BA1:MTCY21B4 | 39150 | Z80108 | Mycobacterium tuberculosis H37Rv complete genome; segment 62/162. | Mycobacterium tuberculosis | 63,017 | 23-Jun-98 |
| | | GB_PAT:I32742 | 5589 | I32742 | Sequence 1 from patent U.S. Pat. No. 5589355. | Unknown. | 66,077 | 6-Feb-97 |
| | | EM_BA1:AB003693 | 5589 | AB003693 | Corynebacterium ammoniagenes DNA for rib operon, complete cds. | Corynebacterium | 66,077 | 03-OCT- |
| | | | | | | ammoniagenes | | 1997 (Rel. |
| | | | | | | | | 52, Created) |
| rxa02260 | 354 | GB_BA1:CORPEPC | 4885 | M25819 | C. glutamicum phosphoenolpyruvate carboxylase gene, complete cds. | Corynebacterium glutamicum | 100,000 | 15-DEC- |
| | | | | | | | | 1995 |
| | | GB_PAT:A09073 | 4885 | A09073 | C. glutamicum ppg gene for phosphoenol pyruvate carboxylase. | Corynebacterium glutamicum | 100,000 | 25-Aug-93 |
| | | GB_BA1:CGL007732 | 4460 | AJ007732 | Corynebacterium glutamicum 3′ ppc gene, secG gene, amt gene, ocd gene and 5′ | Corynebacterium glutamicum | 100,000 | 7-Jan-99 |
| | | | | | soxA gene. | | | |
| rxa02290 | 522 | GB_GSS11:AQ262166 | 588 | AQ262166 | CITBI-E1-2509J2.TF CITBI-E1 Homo sapiens genomic clone 2509J2, genomic | Homo sapiens | 41,505 | 24-OCT- |
| | | | | | survey sequence. | | | 1998 |
| | | GB_HTG5:AC006209 | 233854 | AC006209 | Homo sapiens clone RP11-546D14, *** SEQUENCING IN PROGRESS ***, 85 | Homo sapiens | 40,719 | 19-Nov-99 |
| | | | | | unordered pieces. | | | |
| | | GB_VI:AF107100 | 2335 | AF107100 | Ecotropis obliqua nuclear polyhedrosis virus ecdysteroid UDP-glucosyltransferase | Ecotropis obliqua nuclear | 38,606 | 4-Apr-99 |
| | | | | | gene, complete cds. | polyhedrosis virus | | |
| rxa02291 | 777 | GB_PL1:ATF17M5 | 96475 | AL035678 | Arabidopsis thaliana DNA chromosome 4, BAC clone F17M5 (ESSA project). | Arabidopsis thaliana | 35,195 | 11-MAR- |
| | | | | | | | | 1999 |
| | | GB_HTG4:AC007621 | 335275 | AC007621 | Homo sapiens chromosome 12p12-21.8-27.2 clone RPCI11-757G14, | Homo sapiens | 36,471 | 21-OCT- |
| | | | | | ***SEQUENCING IN PROGRESS ***, 142 unordered pieces. | | | 1999 |
| | | GB_HTG4:AC007621 | 335275 | AC007621 | Homo sapiens chromosome 12p12-21.8-27.2 clone RPCI11-757G14, | Homo sapiens | 36,471 | 21-OCT- |
| | | | | | ***SEQUENCING IN PROGRESS ***, 142 unordered pieces. | | | 1999 |
| rxa02323 | 1047 | GB_PL1:YSKERD2A | 1248 | M34844 | K. lactis ER lumen protein retaining receptor (ERD2) gene, complete cds. | Kluyveromyces lactis | 37,168 | 27-Apr-93 |
| | | GB_PL2:CNS01AFM | 720 | AL112874 | Botrytis cinerea strain T4 cDNA library under conditions of nitrogen deprivation. | Botryotinia fuckeliana | 39,638 | 2-Sep-99 |
| | | GB_PR1:HAAXTRSYV | 6972 | X90840 | H. sapiens mRNA for axonal transporter of synaptic vesicles. | Homo sapiens | 38,454 | 28-MAY- |
| | | | | | | | | 1996 |
| rxa02386 | 582 | GB_OV:AF131057 | 1875 | AF131057 | Gallus gallus substance P receptor (ASPR) mRNA, complete cds. | Gallus gallus | 38,382 | 18-MAY- |
| | | | | | | | | 1999 |
| | | GB_HTG2:AC008225 | 110418 | AC008225 | Drosophila melanogaster chromosome 3 clone BACR03E11 (D818) RPCI-98 | Drosophila melanogaster | 39,236 | 2-Aug-99 |
| | | | | | 03.E.11 map 84C-84D strain y; cn bw sp, *** SEQUENCING | | | |
| | | | | | IN PROGRESS***, 76 unordered pieces. | | | |
| | | GB_EST10:AA142237 | 594 | AA142237 | CK00013.3prime CK Drosophila melanogaster embryo BlueScript Drosophila | Drosophila melanogaster | 36,519 | 29-Nov-98 |
| | | | | | melanogaster cDNA clone CK00013 3prime, mRNA sequence. | | | |
| rxa02388 | 1785 | GB_RO:RNY16563 | 12507 | Y16563 | Rattus norvegicus mRNA for brain-specific synapse-associated protein, Bassoon. | Rattus norvegicus | 35,082 | 11-Aug-98 |
| | | GB_PR4:AF052224 | 15964 | AF052224 | Homo sapiens neuronal double zinc finger | Homo sapiens | 36,270 | 09-DEC- |
| | | | | | protein (ZNF231) mRNA, complete cds. | | | 1998 |
| | | GB_PR1:AB007894 | 5650 | AB007894 | Homo sapiens KIAA0434 mRNA, partial cds. | Homo sapiens | 36,970 | 13-Feb-99 |
| rxa02413 | 615 | GB_PR4:AC007102 | 176258 | AC007102 | Homo sapiens chromosome 4 clone C0162P16 map 4p16, complete sequence. | Homo sapiens | 36,772 | 2-Jun-99 |
| | | GB_HTG3:AC011214 | 183414 | AC011214 | Homo sapiens clone 5_C_3, LOW-PASS SEQUENCE SAMPLING. | Homo sapiens | 36,442 | 03-OCT- |
| | | | | | | | | 1999 |
| | | GB_HTG3:AC011214 | 183414 | AC011214 | Homo sapiens clone 5_C_3, LOW-PASS SEQUENCE SAMPLING. | Homo sapiens | 36,442 | 03-OCT- |
| | | | | | | | | 1999 |
| rxa02416 | 2952 | GB_BA1:MSGB1133CS | 42106 | L78811 | Mycobacterium leprae cosmid B1133 DNA sequence. | Mycobacterium leprae | 65,083 | 15-Jun-96 |
| | | GB_BA1:MTCY06H11 | 38000 | Z85982 | Mycobacterium tuberculosis H37Rv complete genome; segment 73/162. | Mycobacterium tuberculosis | 66,278 | 17-Jun-98 |
| | | GB_BA1:SCC54 | 30753 | AL035591 | Streptomyces coelicolor cosmid C54. | Streptomyces coelicolor | 39,079 | 11-Jun-99 |
| rxa02418 | 690 | GB_BA1:MTCY06H11 | 38000 | Z85982 | Mycobacterium tuberculosis H37Rv complete genome; segment 73/162. | Mycobacterium tuberculosis | 62,899 | 17-Jun-98 |
| | | GB_BA1:MSGB1133CS | 42106 | L78811 | Mycobacterium leprae cosmid B1133 DNA sequence. | Mycobacterium leprae | 66,473 | 15-Jun-96 |
| | | GB_BA1:SCI35 | 40909 | AL031541 | Streptomyces coelicolor cosmid I35. | Streptomyces coelicolor | 35,958 | 9-Sep-98 |
| rxa02429 | 2346 | GB_BA1:MLCB1788 | 39228 | AL008609 | Mycobacterium leprae cosmid B1788. | Mycobacterium leprae | 40,352 | 27-Aug-99 |
| | | GB_BA1:MTCY1A11 | 30850 | Z78020 | Mycobacterium tuberculosis H37Rv complete genome; segment 83/162. | Mycobacterium tuberculosis | 57,417 | 17-Jun-98 |
| | | GB_PL2:AC007153 | 103223 | AC007153 | Arabidopsis thaliana chromosome I BAC F3F20 genomic sequence, complete | Arabidopsis thaliana | 36,104 | 17-MAY- |
| | | | | | sequence. | | | 1999 |
| rxa02436 | 684 | GB_BA1:MTCY10H4 | 39160 | Z80233 | Mycobacterium tuberculosis H37Rv complete genome; segment 2/162. | Mycobacterium tuberculosis | 63,274 | 17-Jun-98 |
| | | GB_BA1:MLCB1770 | 37821 | Z70722 | Mycobacterium leprae cosmid B1770. | Mycobacterium leprae | 62,719 | 29-Aug-97 |
| | | GB_BA1:SCH69 | 35824 | AL079308 | Streptomyces coelicolor cosmid H69. | Streptomyces coelicolor | 40,237 | 15-Jun-99 |
| rxa02445 | 1812 | GB_PR3:HS864I18 | 106018 | AL031293 | Human DNA sequence from clone 864I18 on chromosome 1p36.11-36.33. | Homo sapiens | 37,409 | 23-Nov-99 |
| | | | | | Contains ESTs, STSs, GSSs, genomic marker D1S2728 and a ca repeat | | | |
| | | | | | polymorphism, complete sequence. | | | |
| | | GB_PR3:HS864I18 | 106018 | AL031293 | Human DNA sequence from clone 864I18 on chromosome 1p36.11-36.33. | Homo sapiens | 38,679 | 23-Nov-99 |
| | | | | | Contains ESTs, STSs, GSSs, genomic marker D1S2728 and a ca repeat | | | |
| | | | | | polymorphism, complete sequence. | | | |
| rxa02456 | 741 | GB_BA2:AF144091 | 2900 | AF144091 | Mycobacterium smegmatis catechol 1,2-dioxygenase (catA) gene, partial cds; | Mycobacterium smegmatis | 57,085 | 15-Jul-99 |
| | | | | | muconolactone isomerase (catC) and sigma factor SigH (sigH) genes, complete | | | |
| | | | | | cds; and unknown genes. | | | |
| | | GB_BA1:MTCY7D11 | 22070 | Z95120 | Mycobacterium tuberculosis H37Rv complete genome; segment 138/162. | Mycobacterium tuberculosis | 35,534 | 17-Jun-98 |
| | | GB_STS:G36947 | 418 | G36947 | SHGC-56623 Human Homo sapiens STS cDNA, sequence tagged site. | Homo sapiens | 36,591 | 1-Jan-98 |
| rxa02462 | 1941 | EM_PAT:E09053 | 2538 | E09053 | gDNA encoding secA protein. | Corynebacterium glutamicum | 99,528 | 07-OCT- |
| | | | | | | | | 1997 (Rel. |
| | | | | | | | | 52, Created) |
| | | GB_BA1:MTY20B11 | 36330 | Z95121 | Mycobacterium tuberculosis H37Rv complete genome; segment 139/162. | Mycobacterium tuberculosis | 38,632 | 17-Jun-98 |
| | | GB_BA2:MBU66080 | 4049 | U66080 | Mycobacterium bovis SecA (secA) gene, complete cds. | Mycobacterium bovis | 68,353 | 3-Sep-98 |
| rxa02476 | 1002 | GB_BA1:AB009078 | 2686 | AB009078 | Brevibacterium saccharolyticum gene for L-2.3-butanediol dehydrogenase, | Brevibacterium | 97,309 | 13-Feb-99 |
| | | | | | complete cds. | saccharolyticum | | |
| | | GB_HTG2:AC007933 | 152224 | AC007933 | Homo sapiens chromosome 17 clone hRPC.908_O_12 map 17, | Homo sapiens | 39,959 | 30-Jun-99 |
| | | | | | ***SEQUENCING IN PROGRESS ***, 11 unordered pieces. | | | |
| | | GB_HTG2:AC007933 | 152224 | AC007933 | Homo sapiens chromosome 17 clone hRPC.908_O_12 map 17, | Homo sapiens | 39,959 | 30-Jun-99 |
| | | | | | ***SEQUENCING IN PROGRESS ***, 11 unordered pieces. | | | |
| rxa02502 | 1515 | GB_PR2:AP000548 | 128077 | AP000548 | Homo sapiens genomic DNA, chromosome 22q11.2, Cat Eye Syndrome region, | Homo sapiens | 36,965 | 01-OCT- |
| | | | | | clone: KB556G11. | | | 1999 |
| | | GB_BA1:MSGY348 | 40056 | AD000020 | Mycobacterium tuberculosis sequence from clone y348. | Mycobacterium tuberculosis | 38,198 | 10-DEC- |
| | | | | | | | | 1996 |
| | | GB_PR2:AP000548 | 128077 | AP000548 | Homo sapiens genomic DNA, chromosome 22q11.2, Cat Eye Syndrome region, | Homo sapiens | 35,839 | 01-OCT- |
| | | | | | clone: KB556G11. | | | 1999 |
| rxa02509 | 1994 | GB_BA1:MTCY1A10 | 25949 | Z95387 | Mycobacterium tuberculosis H37Rv complete genome; segment 117/162. | Mycobacterium tuberculosis | 38,806 | 17-Jun-98 |
| | | GB_BA1:MLCL581 | 36225 | Z96801 | Mycobacterium leprae cosmid L581. | Mycobacterium leprae | 38,532 | 24-Jun-97 |
| | | GB_BA1:MTCY1A10 | 25949 | Z95387 | Mycobacterium tuberculosis H37Rv complete genome; segment 117/162. | Mycobacterium tuberculosis | 39,036 | 17-Jun-98 |
| rxa02523 | 942 | GB_BA1:MLCB250 | 40603 | Z97369 | Mycobacterium leprae cosmid B250. | Mycobacterium leprae | 47,284 | 27-Aug-99 |
| | | GB_EST25:AU041363 | 542 | AU041363 | AU041363 Mouse four-cell-embryo cDNA Mus musculus cDNA clone J1001B09 | Mus musculus | 39,180 | 04-DEC- |
| | | | | | 3′, mRNA sequence. | | | 1998 |
| | | GB_EST9:C22241 | 332 | C22241 | C22241 Miyagawa-wase satsuma mandarin orange (M. Omura) Citrus unshiu | Citrus unshiu | 42,638 | 29-Jun-98 |
| | | | | | cDNA clone pcMFRI802.43, mRNA sequence. | | | |
| rxa02557 | 711 | GB_HTG3:AC010964 | 41594 | AC010964 | Homo sapiens chromosome 17 clone 3023_F_18 map 17, | Homo sapiens | 36,234 | 28-Sep-99 |
| | | | | | *** SEQUENCING IN PROGRESS ***, 3 unordered pieces. | | | |
| | | GB_HTG3:AC010964 | 41594 | AC010964 | Homo sapiens chromosome 17 clone 3023_F_18 map 17, | Homo sapiens | 36,234 | 28-Sep-99 |
| | | | | | *** SEQUENCING IN PROGRESS ***, 3 unordered pieces. | | | |
| | | GB_PR2:AC000003 | 122228 | AC000003 | Homo sapiens chromosome 17, clone 104H12, complete sequence. | Homo sapiens | 36,222 | 07-OCT- |
| | | | | | | | | 1997 |
| rxa02563 | 855 | GB_GSS14:AQ570921 | 491 | AQ570921 | HS_5356_B1_H12_T7A RPCI-11 Human Male BAC Library Homo sapiens | Homo sapiens | 35,191 | 1-Jun-99 |
| | | | | | genomic clone Plate = 932 Col = 23 Row = P, genomic survey sequence. | | | |
| | | GB_EST27:AI425057 | 501 | AI425057 | tg50g05.x1 Soares_NFL_T_GBC_S1 Homo sapiens cDNA | Homo sapiens | 38,723 | 30-MAR- |
| | | | | | clone IMAGE: 2112248 3′, mRNA sequence. | | | 1999 |
| | | GB_EST6:N63837 | 469 | N63837 | za26h12.s1 Soares fetal liver spleen 1NFLS Homo sapiens cDNA clone | Homo sapiens | 36,725 | 01-MAR- |
| | | | | | IMAGE: 293735 3′, mRNA sequence. | | | 1996 |
| rxa02590 | 1059 | GB_PAT:I92041 | 858 | I92041 | Sequence 8 from patent U.S. Pat. No. 5726299. | Unknown. | 34,837 | 01-DEC- |
| | | | | | | | | 1998 |
| | | GB_PAT:I78752 | 858 | I78752 | Sequence 8 from patent U.S. Pat. No. 5693781. | Unknown. | 34,837 | 3-Apr-98 |
| | | GB_HTG2:AC006936 | 221373 | AC006936 | Drosophila melanogaster chromosome 3 clone BACR48I01 (D484) | Drosophila melanogaster | 36,103 | 2-Aug-99 |
| | | | | | RPCI-98 48.I.1 map 93E4-93E7 strain y; cn bw sp, | | | |
| | | | | | *** SEQUENCING IN PROGRESS ***, 63 unordered pieces. | | | |
| rxa02608 | 2094 | GB_BA1:CGCOP1G | 2547 | X66078 | C. glutamicum cop1 gene for PS1. | Corynebacterium glutamicum | 99,140 | 30-Jun-93 |
| | | GB_PAT:A26027 | 2547 | A26027 | C. melassecola gene for extracellular antigen PS1. | Corynebacterium melassecola | 99,045 | 2-Apr-95 |
| | | GB_HTG6:AC008180_2 | 110000 | AC008180 | Homo sapiens clone RP11-292L5, *** SEQUENCING IN PROGRESS ***, 152 | Homo sapiens | 35,990 | AC008180 |
| | | | | | unordered pieces. | | | |
| rxa02625 | 886 | GB_BA1:MTV012 | 70287 | AL021287 | Mycobacterium tuberculosis H37Rv complete genome; segment 132/162. | Mycobacterium tuberculosis | 39,135 | 23-Jun-99 |
| | | GB_BA1:SC8D9 | 38681 | AL035569 | Streptomyces coelicolor cosmid 8D9. | Streptomyces coelicolor | 65,537 | 26-Feb-99 |
| | | GB_BA1:MLCB637 | 44882 | Z99263 | Mycobacterium leprae cosmid B637. | Mycobacterium leprae | 63,995 | 17-Sep-97 |
| rxa02671 | 702 | GB_EST38:AW029724 | 634 | AW029724 | EST272979 tomato call U.S. Pat. No., TAMU Lycopersicon esculentum | Lycopersicon esculentum | 34,750 | 15-Sep-99 |
| | | | | | cDNA clone cLEC28I17 similar to beta-ketoacyl-ACP synthase, putative, | | | |
| | | | | | mRNA sequence. | | | |
| | | GB_GSS6:AQ843663 | 631 | AQ843663 | nbxb0024L12r CUGI Rice BAC Library Oryza sativa genomic clone | Oryza sativa | 41,971 | 04-OCT- |
| | | | | | nbxb0024L12r, genomic survey sequence. | | | 1999 |
| | | GB_EST38:AW029724 | 634 | AW029724 | EST272979 tomato call U.S. Pat. No., TAMU Lycopersicon esculentum cDNA | Lycopersicon esculentum | 38,760 | 15-Sep-99 |
| | | | | | clone cLEC28I17 similar to beta-ketoacyl-ACP synthase, putative, | | | |
| | | | | | mRNA sequence. | | | |
| rxa02686 | 1260 | GB_BA1:CORPHEA | 1088 | M13774 | C. glutamicum pheA gene encoding prephenate dehydratase, complete cds. | Corynebacterium glutamicum | 44,279 | 26-Apr-93 |
| | | GB_PAT:E06110 | 948 | E06110 | DNA encoding prephenate dehydratase. | Corynebacterium glutamicum | 43,836 | 29-Sep-97 |
| | | GB_PAT:E04484 | 948 | E04484 | DNA encoding prephenate dehydratase. | Corynebacterium glutamicum | 43,836 | 29-Sep-97 |
| rxa02692 | 1389 | GB_BA1:MTCY1A6 | 37751 | Z83864 | Mycobacterium tuberculosis H37Rv complete genome; segment 159/162. | Mycobacterium tuberculosis | 35,699 | 17-Jun-98 |
| | | GB_PAT:I60487 | 1260 | I60487 | Sequence 3 from patent U.S. Pat. No. 5656470. | Unknown. | 67,383 | 07-OCT- |
| | | | | | | | | 1997 |
| | | GB_BA1:MSGY409 | 41321 | AD000017 | Mycobacterium tuberculosis sequence from clone y409. | Mycobacterium tuberculosis | 63,413 | 10-DEC- |
| | | | | | | | | 1996 |
| rxa02726 | 3057 | GB_BA1:MTCY48 | 35377 | Z74020 | Mycobacterium tuberculosis H37Rv complete genome; segment 69/162. | Mycobacterium tuberculosis | 65,390 | 17-Jun-98 |
| | | GB_PAT:AR009609 | 3905 | AR009609 | Sequence 1 from patent U.S. Pat. No. 5756327. | Unknown. | 65,160 | 04-DEC- |
| | | | | | | | | 1998 |
| | | GB_PAT:AR009610 | 1487 | AR009610 | Sequence 3 from patent U.S. Pat. No. 5756327. | Unknown. | 63,792 | 04-DEC- |
| | | | | | | | | 1998 |
| rxa02731 | 2220 | GB_BA1:MTCY01B2 | 35938 | Z95554 | Mycobacterium tuberculosis H37Rv complete genome; segment 72/162. | Mycobacterium tuberculosis | 70,069 | 17-Jun-98 |
| | | GB_BA1:MSGB1133CS | 42106 | L78811 | Mycobacterium leprae cosmid B1133 DNA sequence. | Mycobacterium leprae | 69,559 | 15-Jun-96 |
| | | GB_BA1:MLUVRB | 2286 | X12578 | Micrococcus luteus gene homologoous to E. coliu vrB gene. | Micrococcus luteus | 63,361 | 12-Sep-93 |
| rxa02742 | 2472 | GB_GSS12:AQ364217 | 467 | AQ364217 | nbxb0060L21f CUGI Rice BAC Library Oryza sativa genomic | Oryza sativa | 37,337 | 3-Feb-99 |
| | | | | | clone nbxb0060L21f, genomic survey sequence. | | | |
| | | GB_GSS12:AQ364217 | 467 | AQ364217 | nbxb0060L21f CUGI Rice BAC Library Oryza sativa genomic | Oryza sativa | 39,123 | 3-Feb-99 |
| | | | | | clone nbxb0060L21f, genomic survey sequence. | | | |
| rxa02748 | 1764 | GB_BA1:CAJ10319 | 5368 | AJ010319 | Corynebacterium glutamicum amtP, glnB, glnD genes and partial ftsY and srp | Corynebacterium glutamicum | 99,888 | 14-MAY- |
| | | | | | genes. | | | 1999 |
| | | GB_BA1:MTCY338 | 29372 | Z74697 | Mycobacterium tuberculosis H37Rv complete genome; segment 127/162. | Mycobacterium tuberculosis | 38,016 | 17-Jun-98 |
| | | GB_BA1:MSGB32CS | 36404 | L78818 | Mycobacterium leprae cosmid B32 DNA sequence. | Mycobacterium leprae | 62,730 | 15-Jun-96 |
| rxa02788 | 2787 | GB_BA1:MTCYW318 | 2803 | Z97051 | Mycobacterium tuberculosis H37Rv complete genome; segment 112/162. | Mycobacterium tuberculosis | 39,294 | 17-Jun-98 |
| | | GB_BA1:MSGB937CS | 38914 | L78820 | Mycobacterium leprae cosmid B937 DNA sequence. | Mycobacterium leprae | 60,729 | 15-Jun-96 |
| | | GB_BA1:MLCB1259 | 38807 | AL023591 | Mycobacterium leprae cosmid B1259. | Mycobacterium leprae | 66,993 | 27-Aug-99 |
| rxa02837 | 274 | GB_BA2:PDU08864 | 2215 | U08864 | Paracoccus denitrificans phosphate acetyltransferase (pta) gene, | Paracoccus denitrificans | 73,723 | 30-Nov-95 |
| | | | | | complete cds, and insertion sequence IS1248a, complete sequence. | | | |
| | | GB_BA1:PDU08856 | 1393 | U08856 | Paracoccus denitrificans insertion sequence IS1248b, complete sequence. | Paracoccus denitrificans | 73,723 | 30-Nov-95 |
| | | GB_BA1:ZMO009974 | 4494 | AJ009974 | Zymomonas mobilis genomic DNA clone encoding ORF1 to 4. | Zymomonas mobilis | 37,500 | 3-Aug-99 |
| rxs03207 | 1123 | GB_BA1:BLSIGBGN | 2906 | Z49824 | B. lactofermentum orf1 gene and sigB gene. | Corynebacterium glutamicum | 99,555 | 25-Apr-96 |
| | | GB_BA1:MTCY05A6 | 38631 | Z96072 | Mycobacterium tuberculosis H37Rv complete genome; segment 120/162. | Mycobacterium tuberculosis | 65,474 | 17-Jun-98 |
| | | GB_BA1:MTU10059 | 5900 | U10059 | Mycobacterium tuberculosis H37Rv sigma factor MysA (mysA) and sigma factor | Mycobacterium tuberculosis | 65,474 | 30-Jan-96 |
| | | | | | MysB (mysB) genes, complete cds. | | | |

What is claimed:

1. An isolated nucleic acid molecule selected from the group consisting of
a) an isolated nucleic acid molecule comprising the nucleotide sequence of SEQ ID NO:499, or a complement thereof;
b) an isolated nucleic acid molecule which encodes a polypeptide comprising the amino acid sequence of SEQ ID NO:500, or a complement thereof;
c) an isolated nucleic acid molecule which encodes a naturally occurring allelic variant of a polypeptide comprising the amino acid sequence of SEQ ID NO:500, or a complement thereof;
d) an isolated nucleic acid molecule comprising a nucleotide sequence which is at least 50% identical to the entire nucleotide sequence of SEQ ID NO:499, or a complement thereof; and
e) an isolated nucleic acid molecule comprising a fragment of at least 15 contiguous nucleotides of the nucleotide sequence of SEQ ID NO:499, or a complement thereof.

2. An isolated nucleic acid molecule comprising the nucleic acid molecule of claim 1 and a nucleotide sequence encoding a heterologous polypeptide.

3. A vector comprising the nucleic acid molecule of claim 1.

4. The vector of claim 3, which is an expression vector.

5. A host cell transfected with the expression vector of claim 4.

6. The host cell of claim 5, wherein said cell is a microorganism.

7. The host cell of claim 6, wherein said cell belongs to the genus Corynebacterium or Brevibacterium.

8. A method of producing a polypeptide comprising culturing the host cell of claim 5 in an appropriate culture medium to, thereby, produce the polypeptide.

9. A method for producing a fine chemical, comprising culturing the cell of claim 5 such that the fine chemical is produced.

10. The method of claim 9, wherein said method further comprises the step of recovering the fine chemical from said culture.

11. The method of claim 9, wherein said cell belongs to the genus Corynebacterium or Brevibacterium.

12. The method of claim 9, wherein said cell is selected from the group consisting of Corynebacterium glutamicum, Corynebacterium herculis, Corynebacterium, lilium, Corynebacterium acetoacidophilum, Corynebacterium acetoglutamicum, Corynebacterium acetophilum, Corynebacterium ammoniagenes, Corynebacterium fujiokense, Corynebacterium nitrilophilus, Brevibacterium ammoniagenes, Brevibacterium butanicum, Brevibacterium divaricatum, Brevibacteriumflavum, Brevibacterium healii, Brevibacterium ketoglutamicum, Brevibacterium ketosoreductum, Brevibacterium lactofermentum, Brevibacterium linens, Brevibacterium paraffinolyticum, and those strains set forth in Table 3.

13. The method of claim 9, wherein expression of the nucleic acid molecule from said vector results in modulation of production of said fine chemical.

14. The method of claim 9, wherein said fine chemical is selected from the group consisting of organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

15. The method of claim 9, wherein said fine chemical is an amino acid selected from the group consisting of lysine, glutamate, glutamine, alanine, aspartate, glycine, serine, threonine, methionine, cysteine, valine, leucine, isoleucine, arginine, proline, histidine, tyrosine, phenylalanine, and tryptophan.

16. An isolated polypeptide selected from the group consisting of
a) an isolated polypeptide comprising the amino acid sequence of SEQ ID NO:500;
b) an isolated polypeptide comprising a naturally occurring allelic variant of a polypeptide comprising the amino acid sequence of SEQ ID NO:500;
c) an isolated polypeptide which is encoded by a nucleic acid molecule comprising the nucleotide sequence of SEQ ID NO:499;
d) an isolated polypeptide which is encoded by a nucleic acid molecule comprising a nucleotide sequence which is at least 50% identical to the entire nucleotide sequence of SEQ ID NO:499;
e) an isolated polypeptide comprising an amino acid sequence which is at least 50% identical to the entire amino acid sequence of SEQ ID NO:500; and
f) an isolated polypeptide comprising a fragment of a polypeptide comprising the amino acid sequence of SEQ ID NO:500, wherein said polypeptide fragment maintains a biological activity of the polypeptide comprising the amino sequence.

17. The isolated polypeptide of claim 16, further comprising heterologous amino acid sequences.

18. A method for diagnosing the presence or activity of Corynebacterium diphtheriae in a subject, comprising detecting the presence of at least one of the nucleic acid molecules of claim 1, thereby diagnosing the presence or activity of Corynebacterium diphtheriae in the subject.

19. A method for diagnosing the presence or activity of Corynebacterium diphtheriae in a subject, comprising detecting the presence of at least one of the polypeptide molecules of claim 16, thereby diagnosing the presence or activity of Corynebacterium diphtheriae in the subject.

20. A host cell comprising a nucleic acid molecule selected from the group consisting of
a) the nucleic acid molecule of SEQ ID NO:499, wherein the nucleic acid molecule is disrupted by at least one technique selected from the group consisting of a point mutation, a truncation, an inversion, a deletion, an addition, a substitution and homologous recombination;
b) the nucleic acid molecule of SEQ ID NO:499, wherein the nucleic acid molecule comprises one or more nucleic acid modifications as compared to the sequence of SEQ ID NO:499, wherein the modification is selected from the group consisting of a point mutation, a truncation, an inversion, a deletion, an addition and a substitution; and
c) the nucleic acid molecule of SEQ ID NO:499, wherein the regulatory region of the nucleic acid molecule is modified relative to the wild-type regulatory region of the molecule by at least one technique selected from the group consisting of a point mutation, a truncation, an inversion, a deletion, an addition, a substitution and homologous recombination.
